SEQUENCE LISTING (28 sequences)

SEQ ID NO: 1 - protein, 78 aa, Homo sapiens
SGVTCADGPCFNGGLCVGGADPDSAYICHCPPGFQGSNCEKRVDRCSLQPCRNGGLCLDLGHALRCRCRAGFAGPRCE

SEQ ID NO: 2 - protein, 40 aa, Homo sapiens
SGVTCADGPCFNGGLCVGGADPDSAYICHCPPGFQGSNCE

SEQ ID NO: 3 - protein, 37 aa, Homo sapiens
DLDDCAGRACANGGTCVEGGGAHRCSCALGFGGRDCR

SEQ ID NO: 4 - protein, 37 aa, Homo sapiens
RADPCAARPCAHGGRCYAHFSGLVCACAPGYMGARCE

SEQ ID NO: 5 - protein, 4 aa, synthetic construct
GGGG

SEQ ID NO: 6 - protein, 5 aa, synthetic construct
GGGGS

SEQ ID NO: 7 - protein, 5 aa, synthetic construct
GGGGQ

SEQ ID NO: 8 - protein, 6 aa, synthetic construct
SGGGGS

SEQ ID NO: 9 - protein, 6 aa, synthetic construct
PGGGGS

SEQ ID NO: 10 - protein, 6 aa, synthetic construct
PGGDGS

SEQ ID NO: 11 - protein, 9 aa, synthetic construct
GGGGSGGGS

SEQ ID NO: 12 - protein, 10 aa, synthetic construct
GGGGSGGGGS

SEQ ID NO: 13 - protein, 15 aa, synthetic construct
GGGGSGGGGSGGGGS

SEQ ID NO: 14 - protein, 241 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 15 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 16 - protein, 982 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 470 aa, synthetic construct
MDMRVPAQLLGLLLLWLRGARCQVQLVESGGGAVQPGRSLRLSCAASGFTFSNYGMHWVRQAPGKGLEWVAVISHHGSSKYYARSVKGRFTISRDNSKNTLYLEMNSLRAEDTAVYYCARDWWELVFDYWGQGTLVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQDSLSLSPGK

SEQ ID NO: 18 - protein, 241 aa, synthetic construct
MDMRVPAQLLGLLLLWLRGARCDIVMTQTPLSLSVTPGQPASISCKSSQSLLHSDGKTFLYWYLQKPGQPPQLLIYEVSNRFSGVPDRFSGSGSGTDFTLKISRVEAEDVGVYYCLQGIHLPFTFGPGTKVEIKRTVAAPSVFIFPPSDEQLKSGTASVVCLLNNFYPREAKVQWKVDNALQSGNSQESVTEQDSKDSTYSLESTLTLSKADYEKHKVYACEVTHQGLSSPVTKSFNRGEC

SEQ ID NO: 19 - protein, 477 aa, synthetic construct
MDMRVPAQLLGLLLLWLRGARCEVQLVESGGGLVQPGGSLKLSCAASGFTFNKYAINWVRQAPGKGLEWVARIRSKYNNYATYYADAVKDRFTISRDDSKNTVYLQMNNLKTEDTAVYYCARAENIGTSYISYWAYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 20 - protein, 234 aa, synthetic construct
MAWALLLLTLLTQGTGSWAQTVVTQEPSLTVSPGGTVTITCGSSTGAVTSGNYPNWVQKKPGQAPRGLIGGTKFLAPGTPARFSGSLSGGKAALTLSGVQPEDEAEYYCVLWYSNRWVFGSGTKLTVLGQPKAAPSVTLFPPSSEELQANKATLVCLISDFYPGAVTVAWKADSSPVKAGVETTTPSKQSNNKYAAKSYLSLTPEQWKSHRSYSCQVTHEGSTVEKTVAPTECS

SEQ ID NO: 21 - protein, 470 aa, synthetic construct
MDMRVPAQLLGLLLLWLRGARCQVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQDSLSLSPGK

SEQ ID NO: 22 - protein, 238 aa, synthetic construct
MMDMRVPAQLLGLLLLWLRGARCEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIKRTVAAPSVFIFPPSDEQLKSGTASVVCLLNNFYPREAKVQWKVDNALQSGNSQESVTEQDSKDSTYSLESTLTLSKADYEKHKVYACEVTHQGLSSPVTKSFNRGEC

SEQ ID NO: 23 - protein, 467 aa, synthetic construct
MDMRVPAQLLGLLLLWLRGARCQVQLVESGGGAVQPGRSLRLSCAASGFTFSNYGMHWVRQAPGKGLEWVAVISHHGSSKYYARSVKGRFTISRDNSKNTLYLEMNSLRAEDTAVYYCARDWWELVFDYWGQGTLVTVSSASTKGPSVFPLAPCSRSTSESTAALGCLVKDYFPEPVTVSWNSGALTSGVHTFPAVLQSSGLYSLKSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 477 aa, synthetic construct
MDMRVPAQLLGLLLLWLRGARCEVQLVESGGGLVQPGGSLKLSCAASGFTFNKYAINWVRQAPGKGLEWVARIRSKYNNYATYYADAVKDRFTISRDDSKNTVYLQMNNLKTEDTAVYYCARAGNFGSSYISYWAYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 25 - protein, 477 aa, synthetic construct; identical to SEQ ID NO: 24

SEQ ID NO: 26 - protein, 505 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLTLSCAASSSSVSLLSLAWYRQAPGKKRELVAGISDDGSIVYMDSVKGRFTISRDNAKNSVYLQMNSLRAEDTAVYYCYAYSWITRSPYWGQGTLVTVSSHHHHHH

SEQ ID NO: 27 - protein, 505 aa, synthetic construct
EVQLVESGGGLVQPGGSLTLSCAASSSSVSLLSLAWYRQAPGKKRELVAGISDDGSIVYMDSVKGRFTISRDNAKNSVYLQMNSLRAEDTAVYYCYAYSWITRSPYWGQGTLVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 28 - protein, 27 aa, Homo sapiens
FPVHPDGASALPAAPPGLRPGDPQRYL

(19) United States
(12) Patent Application Publication
(10) Pub. No.: US 2026/0201027 A1
(43) Pub. Date: Jul. 16, 2026

(54) METHODS FOR TREATING DLL3-EXPRESSING CANCER

(71) Applicant: AMGEN INC., Thousand Oaks, CA (US)

(72) Inventors: Nooshin HASHEMI SADRAEI, Calabasas, CA (US); Mukul MINOCHA, Oak Park, CA (US); Amanda GOLDRICK, Santa Monica, CA (US); Xi CHEN, Camarillo, CA (US); Mira KISTLER, Calabasas, CA (US); Chia-Hsin JU, Belmont, CA (US); Aaron ELLISON, Thousand Oaks, CA (US); Amrita PATI, Belmont, CA (US); Beate SABLE, Newbury Park, CA (US)

(73) Assignee: AMGEN INC., Thousand Oaks, CA (US)

(21) Appl. No.: 19/135,304

(22) PCT Filed: Dec. 7, 2023
(86) PCT No.: PCT/US2023/082826

§ 371 Date: Jun. 3, 2025

Related U.S. Application Data
(60) Provisional application No. 63/386,606, filed on Dec. 8, 2022.
(60) Provisional application No. 63/579,150, filed on Aug. 28, 2023.

(51) Int. Cl. C07K 16/28; A61K 39/00; A61K 39/395; A61K 45/06; A61P 35/00

(52) U.S. Cl. CPC C07K 16/28; A61K 39/3955; A61K 45/06; A61P 35/00; C07K 16/2809; A61K 2039/505; A61K 2039/545; C07K 2317/31

(57) ABSTRACT

The disclosure provides methods for treating small cell lung cancer (SCLC) in a subject expressing human delta-like ligand 3 (DLL3) protein. The methods comprise administering to the subject an antigen-binding molecule comprising at least a first binding domain that binds to human DLL3, wherein at least 25% of the SCLC cells express DLL3, or wherein at least 25% of the SCLC cells express DLL3 at an intensity equal to or greater than 2+ as determined by an IHC assay.

### FIELD

The disclosure relates to methods for treating DLL3-expressing cancer, such as small cell lung cancer (SCLC).

### BACKGROUND

Small cell lung cancer (SCLC) is an aggressive form of lung cancer with a poor prognosis and limited therapeutic options, representing about 13% of all newly diagnosed lung cancers, with more than 235,000 adults receiving a diagnosis of SCLC in the U.S. in 2021. Survival rates have remained low for several decades, with only 7% of SCLC patients surviving five years, in a large part due to the lack of new therapies to combat this form of lung cancer. Most patients present with extensive-stage disease, while about a third of patients present with limited stage disease, defined by the presence of tumors in only one side of the chest and that fit in a single radiation field. Disseminated, metastatic tumors with lymphoma-like characteristics are a hallmark of SCLC. The first known diagnosis of SCLC patients described it as a disease of the lymphatic system, and SCLC was not recognized as lung cancer until 1926, which highlights the unique nature of SCLC tumors as compared to other solid tumors.

Patients typically respond well to the current standard of care, which includes chemotherapy combined with thoracic radiation therapy (TRT), but invariably quickly relapse with chemoresistant disease, for which no therapeutic options are currently available. Recently, the addition of the anti-PD-L1 antibody atezolizumab (TECENTRIQ®) to carboplatin and etoposide chemotherapy demonstrated an improvement in overall survival (OS) in the first-line setting, leading to the approval of this regimen by the United States Food and Drug Administration (FDA) for first-line treatment of extensive-stage SCLC. Despite these therapeutic advances, prognosis in the relapsed refractory (RR) setting is extremely poor, with rapid disease progression and short median survival of less than six months. Furthermore, SCLC patients have high rates of comorbidities, including hypertension, cardiac disease, diabetes and paraneoplastic syndromes. These, coupled with the typically advanced age of SCLC patients, impact the ability of patients to endure harsh chemotherapy regimens, further limiting treatment options.

Delta-like ligand 3 (DLL3) is an inhibitory Notch ligand that is highly expressed in SCLC and other neuroendocrine tumors but minimally expressed in normal tissues. In one study, approximately 86% of SCLC tumors analyzed showed evidence of DLL3 expression by RNA-seq (Giffin et al., Clin. Cancer Res., 27 (5): 1526-1537 (2021). doi: 10.1158/1078-0432.CCR-20-2845). In contrast, only a few normal cell types have been shown to express DLL3 (e.g., neurons, pancreatic islet cells, and pituitary cells), and such expression was predominantly cytoplasmic. Recent studies have reported that DLL3 is also expressed in other tumor types of neuroendocrine origin, including melanoma, glioblastoma multiforme, neuroendocrine prostate cancer (NEPC), and large cell neuroendocrine lung tumors.

Therapies targeting DLL3 are being investigated for treating DLL3-expressing cancers. For example, rovalpituzumab tesirine (Rova-T) is an antibody-drug conjugate (ADC) containing a DLL3-targeting antibody tethered to the cytotoxic agent pyrrolobenzodiazepine via of a protease-cleavable linker. Despite promising results in early clinical trials, development of Rova-T was discontinued after it demonstrated limited efficacy and higher rates of certain toxicities in phase 3 trials (Blackhall et al., J. Thoracic Oncology, 16 (9): 1547-1558 (2021); and Upetry et al., J. Thoracic Oncology, 16 (9): 1429-1433 (2021)). The therapeutic effects of ADCs are mediated by the cytotoxic/cytostatic agent to which the antibody is conjugated, and ADCs do not generally engage, recruit, and/or activate cells of the immune system. As such, DLL3 remains a promising target for immunotherapeutics that, for example, engage and activate cytotoxic T cells.

There remains a need for methods for identifying and treating patient populations that would benefit from DLL3-targeted immunotherapeutics.

### BRIEF SUMMARY

The disclosure provides a method of treating small cell lung cancer (SCLC) in a subject, which method comprises administering to the subject a bispecific antigen-binding molecule comprising at least a first binding domain that binds to human delta-like ligand 3 (DLL3), wherein at least 25% of the SCLC cells express DLL3.

In some aspects of the method, at least 50% or at least 75% of the SCLC cells express DLL3.

In some aspects of the method, DLL3 expression is determined using an immunohistochemical (IHC) assay.

In some aspects of the method, DLL3 expression is determined using Formalin Fixed Paraffin Embedded (FFPE) tissue specimens from the subject.

In some aspects of the method, the SCLC has progressed or recurred in the subject following a platinum-based treatment.

In some aspects of the method, the SCLC has progressed or recurred in the subject following a platinum-based treatment in combination with etoposide, and optionally a PD-L1 inhibitor.

In some aspects of the method, the subject (i) has completed up to two cycles of platinum-based treatment in combination with etoposide and optionally a PD-L1 inhibitor, or (ii) has completed four to six cycles of platinum-based treatment in combination with etoposide and optionally a PD-L1 inhibitor, and has not experienced disease progression.

In some aspects, the method further comprises administering to the subject a PD-L1 inhibitor and optionally a chemotherapeutic agent.

In some aspects of the method, the bispecific antigen-binding molecule comprises a second binding domain that binds to human CD3.

In some aspects of the method, the bispecific antigen-binding molecule is a protein.

In some aspects of the method, the bispecific antigen-binding molecule comprises an antibody, a single chain variable fragment (scFv), tandem single-chain variable fragments (scFv)2, a bispecific T cell engager (BiTE®) molecule, or a heteromultimer.

In some aspects of the method, the bispecific antigen-binding molecule comprises the amino acid sequences of SEQ ID NO: 14 and SEQ ID NO: 15.

In some aspects of the method, the bispecific antigen-binding molecule is tarlatamab.

In some aspects of the method, the bispecific antigen-binding molecule comprises a first heterodimer that binds to human DLL3 and a second heterodimer that binds to human CD3, wherein (a) the first heterodimer comprises a heavy chain (HC) comprising the amino acid sequence of SEQ ID NO: 17 and a light chain (LC) comprising the amino acid sequence of SEQ ID NO: 18; and (b) the second heterodimer comprises a heavy chain comprising the amino acid sequence of SEQ ID NO: 19 and a light chain comprising the amino acid sequence of SEQ ID NO: 20.

In some aspects, the method increases progression free survival (PFS) of the subject as compared to a second SCLC subject comprising less than 50% of SCLC cells expressing DLL3 and treated with the same antigen-binding molecule.

In some aspects, the method increases one or more of Overall Survival (OS), Objective Response Rate (ORR), and/or Disease Control Rate (DCR) and Duration of Response (DOR) as compared to the second subject.

In some aspects, the method results in an Objective Response Rate (ORR) in the subject that is greater than about 35%.

The disclosure also provides a method of treating SCLC in a subject, which method comprises administering to the subject a bispecific antigen-binding molecule comprising at least a first binding domain that binds to human DLL3, wherein the SCLC has a DLL3 expression level in which at least 25% of the SCLC cells express DLL3 at an intensity equal to or greater than 2+ as determined by an IHC assay.

In some aspects of the method, the SCLC has a DLL3 expression level in which at least 50% or at least 75% of the SCLC cells express DLL3 at an intensity equal to or greater than 2+ as determined by an IHC assay.

In some aspects of the method, at least 25% of the SCLC cells express DLL3 at an intensity of 2+ or 3+ as determined by the IHC assay.

In some aspects of the method, DLL3 expression is determined using Formalin Fixed Paraffin Embedded (FFPE) tissue specimens from the subject.

In some aspects of the method, the SCLC has progressed or recurred in the subject following a platinum-based treatment.

In some aspects of the method, the SCLC has progressed or recurred in the subject following a platinum-based treatment in combination with etoposide, and optionally a PD-L1 inhibitor.

In some aspects of the method, the subject (i) has completed up to two cycles of platinum-based treatment in combination with etoposide and optionally a PD-L1 inhibitor, or (ii) has completed four to six cycles of platinum-based treatment in combination with etoposide and optionally a PD-L1 inhibitor, and has not experienced disease progression.

In some aspects, the method further comprises administering to the subject a PD-L1 inhibitor and optionally a chemotherapeutic agent.

In some aspects of the method, the bispecific antigen-binding molecule comprises a second binding domain that binds to human CD3.

In some aspects of the method, the bispecific antigen-binding molecule is a protein.

In some aspects of the method, the bispecific antigen-binding molecule comprises an antibody, a single chain variable fragment (scFv), tandem single-chain variable fragments (scFv)2, a bispecific T cell engager (BiTE®) molecule, or a heteromultimer.

In some aspects of the method, the bispecific antigen-binding molecule comprises SEQ ID NO: 14 and SEQ ID NO: 15.

In some aspects of the method, the bispecific antigen-binding molecule is tarlatamab.

In some aspects of the method, the bispecific antigen-binding molecule comprises a first heterodimer that binds to human DLL3 and a second heterodimer that binds to human CD3, wherein (a) the first heterodimer comprises a heavy chain (HC) comprising the amino acid sequence of SEQ ID NO: 17 and a light chain (LC) comprising the amino acid sequence of SEQ ID NO: 18; and (b) the second heterodimer comprises a heavy chain comprising the amino acid sequence of SEQ ID NO: 19 and a light chain comprising the amino acid sequence of SEQ ID NO: 20.

In some aspects, the method increases progression free survival (PFS) of the subject compared to a second SCLC subject having a lower DLL3 expression level and treated with the same antigen-binding molecule.

In some aspects, the method increases one or more of OS, ORR, and/or DCR and DOR as compared to the second subject.

In some aspects, the method results in an Objective Response Rate (ORR) in the subject that is greater than about 35%.

In some aspects of the method, the cancer cells are viable cancer cells.

In some aspects of the method, the bispecific antigen-binding molecule is tarlatamab, and tarlatamab is administered at a dose of from 10 mg to 100 mg once every two weeks.

In some aspects of the method, tarlatamab is administered at a dose of 10 mg once every two weeks.

In some aspects of the method, tarlatamab is administered at a dose of 100 mg once every two weeks.

In some aspects, the method comprises administering tarlatamab at a dose of 10 mg to 100 mg once a week on weeks 1, 2, and 3 prior to administering tarlatamab once every two weeks.

In some aspects of the method, the bispecific antigen-binding molecule is tarlatamab, and tarlatamab is administered at a dose of from 10 mg to 100 mg twice every three weeks.

In some aspects of the method, tarlatamab is administered at a dose of 10 mg, 30 mg, or 100 mg twice every three weeks.

In some aspects of the method, tarlatamab is administered on day 1 and day 8 of a 21-day cycle.

In some aspects of the method, the bispecific antigen-binding molecule is tarlatamab, and tarlatamab is administered at a dose of from 20 mg to 200 mg once every three weeks.

In some aspects of the method, tarlatamab is administered at a dose of from 20 mg to 100 mg once every three weeks.

In some aspects of the method, tarlatamab is administered at a dose of from 100 mg to 200 mg once every three weeks.

In some aspects of the method, tarlatamab is administered at a dose of 20 mg, 60 mg, 100 mg, or 200 mg.

In some aspects of the method, tarlatamab is administered on day one of a 21-day cycle.

In some aspects, the method comprises administering tarlatamab at a dose of 10 mg to 100 mg once a week on weeks 1 and 2 prior to administering tarlatamab once every three weeks.

In some aspects of the method, the FFPE tissue specimens are prepared from core needle biopsy tissue samples.

In some aspects of the method, the subject is a human. In some embodiments, the human had at least one prior treatment of the cancer and relapsed. In some embodiments, the at least one prior treatment is a platinum-based chemotherapy and etoposide, and optionally an anti-PD-L1 antibody. In some embodiments, the human had no prior systemic treatment of the cancer.

### BRIEF DESCRIPTION OF THE DRAWINGS

FIG. 1A is a graph showing the best percent change from baseline in tumor burden (defined by the sum of the longest diameters (SLD) of all target lesions) in 94 patients whose data cut-off date was at least 9 weeks after the first dose date and for whom postbaseline tumor data were available. CR denotes complete response, PR partial response, SD stable disease, and NE not evaluable. SD\hat{ } indicates patients had an initial response but did not have confirmation of response on the subsequent scan and PR** indicates patients had an initial PR and still have potential for future confirmative scans. One confirmed subject in cohort 30 had missing sum of diameters for lesion measurement and was not included in the plot. †Step dosing (i.e., 1 mg run-in dose) was used in these cohorts. FIG. 1B is a graph showing the time to response, the duration of treatment, and patient status as of the data cutoff date according to dose of tarlatamab for all patients with confirmed response (N=25).

FIG. 2A shows a Kaplan-Meier curve of progression-free survival for patients whose data cut-off date was at least 9 weeks after the first dose date (N=107). FIG. 2B shows a Kaplan-Meier curve of overall survival for patients whose data cut-off date was at least 9 weeks after the first dose date (N=107).

FIG. 3 is ROC curve reflecting True Positive Rate (TPR) and False Positive Rate (FPR) of enrichment of objective response when FIH subjects (1-100 mg cohorts) are retrospectively examined for selection using DLL3 expression assessed with the Ventana SP347 assay. ROC analysis included 77 subjects enrolled in the 1-100 mg cohorts for whom pre-treatment DLL3 expression readouts were available.

FIG. 4 is graph showing Objective Response Rate (ORR) and Disease Control Rate (DCR) versus percentage of tumor cells expressing DLL3.

FIG. 5A is graph showing the relationship between total DLL3 expression and tumor reduction in FIH cohorts that were dosed with tarlatamab monotherapy with 1 step to target dose as described in Example 1. FIG. 5B is a graph showing the relationship between tumors with 3+DLL3 expression intensity and tumor reduction. BOR denotes best overall response, CR denotes complete response, PR denotes partial response, PD denotes progressive disease, SD denotes stable disease, NE denotes not evaluable, and NA denotes “not available.”

FIGS. 6A-6D are graphs showing peak cytokine levels (6A: IL-6; 6B: IL-8; 6C: IL-10; 6D: TNF-α) trended higher in patients with CRS as compared to patients without CRS. Biomarker evaluable patients (N=86); patients with any grade CRS in Cycle 1 (n=45); patients without CRS (n=40).C1, Cycle 1; CRS, cytokine release syndrome; G, grade.

FIG. 7 is a graph showing a longitudinal analysis of IL-10 expression in patients from the phase I study described in Example 1. IL-10 showed significant elevation above referenced normal range, and was higher in patients with CRS. JT Trend test adjusted P-value=0.049 (significant at 95% confidence); KW test of association adjusted P-value=0.096 (significant at 90% confidence; not significant at 95% confidence). Yellow dotted lines indicate referenced normal range.

FIGS. 8A and 8B are graphs showing analysis of IFN-γ expression in patients from the phase I study described in Example 1. IFN-γ induction was above the physiologic range; induction was similar between those with versus without Cycle 1 CRS. JT Trend test adjusted P-value=0.234 (not significant at 95% confidence); KW test of association adjusted P-value=0.317 (not significant at 90% or at 95% confidence). Yellow dotted lines indicate referenced normal range.

FIG. 9 is a schematic diagram of the clinical study described in Example 2.

FIG. 10 is a waterfall-swimmer plot showing efficacy of tarlatamab in DLL3+NEPC tumors as described in Example 4.

### DETAILED DESCRIPTION

The present disclosure is predicated, at least in part, on the finding that DLL3 is expressed by certain types of cancers. DLL3 was identified as a tumor-associated antigen and a target for T cell-based therapies by analyzing its differential expression in small-cell lung cancer (SCLC) tumors and a large panel of normal tissues (Giffin et al., J Thorac Oncol., 13(10): S971 (2018)). DLL3 overexpression has been observed on the surface of SCLC and large cell neuroendocrine carcinoma (LCNEC) (Rudin et al., Lancet Oncol. 2017; 18:42-51; and Saunders et al., Sci Transl Med., 2015; 7: 302ra136). DLL3 expression also has been observed in cells of other cancers, including pancreatic cancer, gastrointestinal neuroendocrine carcinoma, small cell bladder cancer, neuroendocrine prostate cancer, isocitrate dehydrogenase-mutant glioma, gynecological cancer, and Merkel cell carcinoma (Matsuo et al., Cancer Science, 112:2984-2992 (2021)). Thus, DLL3 is a possible therapeutic target for a variety of cancers.

The present disclosure provides methods of treating DLL3-expressing cancer (e.g., small cell lung cancer (SCLC)) in a subject (e.g., a human subject) which comprises administering to the subject a bispecific antigen-binding molecule comprising at least a first binding domain that binds to human DLL3. Administration of the bispecific antigen-binding molecules described herein to SCLC cancer cells expressing certain levels of DLL3 is advantageously associated with improved therapeutic outcomes (e.g., increased progression free survival (PFS) and objective response rate (ORR)) as compared to SCLC cancer cells with lower or no DLL3 expression treated with the same bispecific antigen-binding molecule.

### Definitions

To facilitate an understanding of the present technology, several terms and phrases are defined below. Additional definitions are set forth throughout the detailed description.

As used herein, the terms “biomarker,” “marker,” or “biological marker” refer to an analyte (e.g., a nucleic acid, DNA, RNA, peptide, protein, or metabolite) that can be objectively measured and evaluated as an indicator for a biological process or condition. In some aspects, an analyte is differentially detectable if it can be distinguished quantitatively or qualitatively in tumor cells compared to a control, e.g., a healthy subject or a subject with SCLC that does not express DLL3.

The term “antigen-binding molecule,” as used herein, refers to a molecule or compound that specifically binds to an antigen. In some embodiments, an anti-binding molecule is a protein. The term “antigen-binding protein,” as used herein, refers to a proteinaceous molecule that specifically binds to an antigen. For example, an antigen-binding protein may comprise an antibody or an antigen-binding fragment thereof. An antigen-binding protein typically comprises the heavy chain variable region (VH) and/or the light chain variable region (VL) of an antibody, or comprises domains derived therefrom. In some embodiments, an antigen-binding protein comprises the minimum structural requirements of an antibody which allow for immunospecific target binding. This minimum requirement may be defined by, for example, the presence of at least three light chain complementarity determining regions (CDRs) (i.e., CDR1, CDR2 and CDR3 of the VL region) and/or three heavy chain CDRs (i.e., CDR1, CDR2 and CDR3 of the VH region), and ideally of all six CDRs. It is within the knowledge of a skilled person where (and in which order) those CDRs are located in the antigen-binding protein.

In certain aspects, the antigen-binding proteins of the present disclosure are “bispecific,” meaning that they are capable of specifically binding to two different antigens. In another aspect, the antigen-binding proteins of the present disclosure may be “trispecific,” meaning that they are capable of specifically binding to three different antigens. In another aspect, the antigen-binding proteins of the present disclosure may be “tetraspecific,” meaning that they are capable of specifically binding to four different antigens.

As used herein, the term “antibody” refers to a whole antibody molecule or a fragment thereof (e.g., fragments such as scFv, Fab, Fab′, and F(ab′)₂), unless specified otherwise; an antibody may be a polyclonal or monoclonal antibody, a chimeric antibody, a humanized antibody, a human antibody, etc. In a native whole antibody, a heavy chain comprises a variable region, VH, and three constant regions, CH1, CH2, and CH3. The VH domain is at the amino-terminus of the heavy chain, and the CH3 domain is at the carboxy-terminus. In a native whole antibody, a light chain comprises a variable region, VL, and a constant region, CL. The variable region of the light chain is at the amino-terminus of the light chain. In a native whole antibody, the variable regions of each light/heavy chain pair typically form the antigen-binding site. The constant regions are typically responsible for effector function.

In a human antibody, CH1 means a region having the amino acid sequence at positions 118 to 215 of the EU index or EU numbering system, which is based on the sequential numbering of the first human IgG1 sequenced (i.e., the “EU antibody”) (Edelman et al., Proc Natl Acad Sci USA, 63 (1): 78-85 (1969)). A highly flexible amino acid region called a “hinge region” exists between CH1 and CH2. CH2 represents a region having the amino acid sequence at positions 231 to 340 of the EU index, and CH3 represents a region having the amino acid sequence at positions 341 to 446 of the EU index.

“CL” represents a constant region of a light chain. In the case of a k chain of a human antibody, CL represents a region having the amino acid sequence at positions 108 to 214 of the EU index. In a A chain, CL represents a region having the amino acid sequence at positions 108 to 215.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen-binding site. The assignment of amino acids to each domain is typically in accordance with the definitions of Kabat et al. (1991) Sequences of Proteins of Immunological Interest (National Institutes of Health, Publication No. 91-3242, vols. 1-3, Bethesda, Md.); Chothia, C., and Lesk, A. M. (1987) J. Mol. Biol., 196:901-917; or Chothia C. et al., Nature, 342:878-883 (1989). In some embodiments, the CDRs of an antigen-binding protein are defined according to the definition of Kabat or Chothia. In the present application, the term “CDR” refers to a CDR from either the light or heavy chain, unless otherwise specified.

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4. In certain embodiments, the antibody is of isotype IgG (e.g., IgG1).

The antibody can be a monoclonal antibody. The term “monoclonal antibody,” as used herein, refers to an antibody produced by a single clone of B lymphocytes that is directed against a single epitope on an antigen. Monoclonal antibodies typically are produced using hybridoma technology, as first described in Kohler and Milstein, Eur. J. Immunol., 5:511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352:624-628 (1991)); and Marks et al., J. Mol. Biol., 222:581-597 (1991)), or produced from transgenic mice carrying a fully human immunoglobulin system (see, e.g., XENOMOUSE™ mouse, Green et al. (1994) Nature Genetics 7:13-21, US 2003-0070185, WO 96/34096, and WO 96/33735). In contrast, “polyclonal” antibodies are antibodies that are secreted by different B cell lineages within an animal. Polyclonal antibodies are a collection of immunoglobulin molecules that recognize multiple epitopes on the same antigen.

The term “chimeric antibody,” as used herein, refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence.

An antibody can be cleaved into fragments by enzymes, such as, e.g., papain and pepsin. Papain cleaves an antibody to produce two Fab fragments and a single Fc fragment. Pepsin cleaves an antibody to produce a F(ab′)2 fragment and a pFc′ fragment. In exemplary aspects, the bispecific antigen-binding protein of the present disclosure comprises an antigen-binding antibody fragment. As used herein, the term “antigen-binding antibody fragment” refers to a portion of an antibody molecule that is capable of binding to the antigen of the antibody and is also known as “antigen-binding fragment” or “antigen-binding portion.” In some embodiments, an antigen-binding antibody fragment is a Fab fragment or a F(ab′)2 fragment.

The architecture of antibodies has been exploited to create a growing range of alternative formats that span a molecular-weight range of at least about 12-150 kDa and has a valency (n) range from monomeric (n=1), to dimeric (n=2), to trimeric (n=3), to tetrameric (n=4), and potentially higher; such alternative formats are referred to herein as “antibody protein products.” Antibody protein products include those based on the full antibody structure and those that mimic antibody fragments which retain full antigen-binding capacity, e.g., single chain variable fragments (scFvs), Fabs, and VHH/VH (discussed below). The smallest antigen-binding antibody fragment that retains its complete antigen-binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment. Both scFv and Fab fragments can be easily produced in host cells, e.g., prokaryotic host cells. Other antibody protein products include tandem single-chain variable fragments (scFv)₂, disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. A peptibody or peptide-Fc fusion is yet another antibody protein product. The structure of a peptibody consists of a biologically active peptide grafted onto an Fc domain. Peptibodies are well-described in the art (see, e.g., Shimamoto et al., mAbs 4 (5): 586-591 (2012)).

The bispecific antigen-binding molecule of the present disclosure may comprise any one of the above-described antibody protein products. In exemplary aspects, the bispecific antigen-binding protein of the present disclosure comprises any one of an scFv, (scFv)₂, Fab, VHH/VH, Fv fragment, ds-scFv, scFab, dimeric antibody, multimeric antibody (e.g., a diabody, triabody, tetrabody), miniAb, peptibody VHH/VH of camelid heavy chain antibody, scFv-single domain mAb, multimeric protein, sdAb, diabody; a triabody; a tetrabody; a bispecific or trispecific antibody, BsIgG, appended IgG, BsAb fragment, or bispecific fusion protein. In certain embodiments, the bispecific antigen-binding protein comprises an (scFv)₂, scFab, scFv-single domain mAb, or multimeric protein. In certain embodiments, the bispecific antigen-binding protein comprises an (scFv)₂ or a multimeric protein.

As used herein, an antigen-binding protein “specifically binds” to a target antigen when it has a significantly higher binding affinity for, and consequently is capable of distinguishing, that antigen, compared to its affinity for other unrelated proteins, under similar binding assay conditions. Antigen-binding proteins that specifically bind an antigen may have an equilibrium dissociation constant (K_{D})≤1×10⁻⁶ M. In exemplary aspects, the K_{D} of the bispecific antigen-binding proteins provided herein is micromolar, nanomolar, picomolar or femtomolar. The antigen-binding protein specifically binds antigen with “high affinity” when the K_{D} is ≤3×10⁻⁸ M. In some embodiments, the bispecific antigen-binding proteins of the disclosure bind to target antigen(s) with a K_{D} of ≤100 nM (e.g., 90 nM, 80 nM, 70 nM, 60 nM, 50 nM, 40 nM, 30 nM, 20 nM, 10 nM, 5 nM, or a range defined by any two of the foregoing values). In other embodiments, the bispecific antigen-binding proteins of the disclosure bind to target antigen(s) with a K_{D} of about 10 nM-30 nM (e.g., about 15 nM, 20 nM, or 25 nM).

Affinity may be determined using a variety of techniques, an example of which is enzyme-linked immunosorbent assay (ELISA). In various embodiments, affinity is determined by a surface plasmon resonance assay (e.g., BIAcore®-based assay). Using this methodology, the association rate constant (kₐ in M⁻¹s⁻¹) and the dissociation rate constant (k_{d} in s⁻¹) can be measured. The equilibrium dissociation constant (K_{D} in M) can then be calculated from the ratio of the kinetic rate constants (k_{d}/kₐ). In some embodiments, affinity may be determined by a kinetic method, such as a Kinetic Exclusion Assay (KinExA) as described in Rathanaswami et al., Analytical Biochemistry, 373:52-60 (2008). Using a KinExA assay, the equilibrium dissociation constant (K_{D} in M) and the association rate constant (ka in M⁻¹s⁻¹) can be measured. The dissociation rate constant (kₐ in s⁻¹) can be calculated from these values (K_{D}×kₐ). In other embodiments, affinity is determined by an equilibrium/solution method. In certain embodiments, affinity is determined by an on-cell binding assay using flow cytometry. In certain embodiments of the disclosure, the bispecific antigen-binding protein specifically binds to target antigen(s) expressed by a mammalian cell (e.g., CHO, HEK 293, Jurkat), with a K_{D} of 20 nM (2.0×10⁻⁸ M) or less, K_{D} of 10 nM (1.0×10⁻⁸ M) or less, K_{D} of 1 nM (1.0×10⁻⁹ M) or less, K_{D} of 500 μM (5.0×10⁻¹⁰ M) or less, K_{D} of 200 μM (2.0×10⁻¹⁰ M) or less, K_{D} of 150 μM (1.50×10⁻¹⁰ M) or less, K_{D} of 125 μM (1.25×10⁻¹⁰ M) or less, K_{D} of 105 μM (1.05×10⁻¹⁰ M) or less, K_{D} of 50 μM (5.0×10⁻¹¹ M) or less, or K_{D} of 20 μM (2.0×10⁻¹¹ M) or less, as determined by a Kinetic Exclusion Assay (Rathanaswami et al., supra). In some embodiments, the bispecific antigen-binding proteins described herein exhibit desirable characteristics such as binding avidity as measured by k_{d} for target antigen(s) of about 10⁻², 10⁻³, 10⁻⁴, 10⁻⁵, 10⁻⁶, 10⁻⁷, 10⁻⁸, 10⁻⁹, 10⁻¹⁰ s⁻¹ or lower (lower values indicating higher binding avidity), and/or binding affinity as measured by K_{D} for target antigen(s) of about 10⁻⁹, 10⁻¹⁰, 10⁻¹¹, 10⁻¹², 10⁻¹³, 10⁻¹⁴, 10⁻¹⁵, 10⁻¹⁶ M or lower (lower values indicating higher binding affinity).

In certain embodiments of the disclosure, bispecific antigen-binding proteins may be multivalent. The valency of the binding protein denotes the number of individual antigen-binding domains within the binding protein. In some embodiments, a bispecific antigen-binding protein may be multivalent. For instance, in certain embodiments, a bispecific antigen-binding protein may be tetravalent by comprising four antigen-binding domains: two antigen-binding domains binding to a first target antigen and two antigen-binding domains binding to a second target antigen. A tetraspecific antigen-binding protein is tetravalent and comprises four antigen-binding domains: one to antigen-binding domain binding to a first target antigen, one antigen-binding domain binding to a second target antigen, one to antigen-binding domain binding to a third target antigen, and one antigen-binding domain binding to a fourth target antigen.

As used herein, the term “antigen-binding domain,” which is used interchangeably with “binding domain,” refers to the region of the antigen-binding protein that contains the amino acid residues that interact with the antigen and confer on the antigen-binding protein its specificity and affinity for the antigen. In some embodiments, the binding domain may be derived from the natural ligands of the target antigen(s). As used herein, the term “target antigen(s)” refers to a first target antigen and/or a second target antigen of a bispecific antigen-binding molecule and also refers to a first target antigen, a second target antigen, a third target antigen, and/or a fourth target antigen of a tetraspecific molecule.

The term “immunoglobulin domain,” as used herein, refers to a peptide comprising an amino acid sequence similar to that of immunoglobulin and comprising approximately 100 amino acid residues including at least two cysteine residues. Examples of immunoglobulin domains include VH, CH1, CH2, and CH3 of an immunoglobulin heavy chain, and VL and CL of an immunoglobulin light chain. In addition, the immunoglobulin domain is found in proteins other than immunoglobulin. Examples of the immunoglobulin domain in proteins other than immunoglobulin include an immunoglobulin domain included in a protein belonging to an immunoglobulin super family, such as a major histocompatibility complex (MHC), CD1, B7, T-cell receptor (TCR), and the like. Any of the immunoglobulin domains can be used as an immunoglobulin domain for the bispecific antigen-binding proteins described herein.

The binding domains that specifically bind to target antigen(s) can be derived a) from known antibodies to these antigens or b) from new antibodies or antibody fragments obtained by de novo immunization methods using the antigen proteins or fragments thereof, by phage display, or other routine methods. The antibodies from which the binding domains for the bispecific antigen-binding proteins are derived can be monoclonal antibodies, polyclonal antibodies, recombinant antibodies, human antibodies, or humanized antibodies. In certain embodiments, the antibodies from which the binding domains are derived are monoclonal antibodies. In these and other embodiments, the antibodies are human antibodies or humanized antibodies and can be of the IgG1-, IgG2-, IgG3-, or IgG4-type. For example, the binding domains may be obtained from or based on an IgG1 monoclonal antibody.

A “multimeric protein,” as used herein, refers to a protein containing more than one separate polypeptide or protein chains associated with each other to form a single protein in vitro or in vivo. A multimeric protein may comprise more than one polypeptide of the same kind to form a “homomultimer.” Alternatively, a multimeric protein may also be composed of more than one polypeptide of distinct sequences to form a “heteromultimer.” Thus, a “heteromultimer” is a molecule comprising at least a first polypeptide and a second polypeptide, wherein the second polypeptide differs in amino acid sequence from the first polypeptide by at least one amino acid residue. The heteromultimer can comprise a “heterodimer” formed by the first and second polypeptide or can form higher order tertiary structures where more than two polypeptides are present.

As used herein, the terms “stability” and “stabilizing” are defined as the maintenance of the chemical or physical integrity and/or bioactivity of the bispecific antigen-binding polypeptide or protein over a period of time. Stabilizing an antigen-binding polypeptide or protein includes the prevention or delay of degradation or deterioration of the antigen-binding polypeptide or protein from its biologically and/or therapeutically active form to an inactive form. Instability may arise from events such as aggregation, denaturation, fragmentation, or chemical modifications such as oxidation, cross-linking, deamidation and reactions with other components featured in the composition comprising the antigen-binding polypeptide or protein.

The stability of an antigen-binding protein or polypeptide may be characterized using known methods in the art, including but not limited to, measurement of biological activity such as antigen-binding activity with immunoassay techniques such as ELISA, or other techniques of determining purity or physical/chemical changes to the antigen-binding protein or polypeptide such as size exclusion chromatography, capillary gel electrophoresis, circular dichroism, or mass spectrometry. Stability is determined by comparison of measurements obtained via these types of characterization methods at an initial time point, such as at the time of formulation or preparation of the composition (i.e., as the case may be, the suspension or dispersion), and those obtained at a later time point, that is, after storage in a given environment or condition.

The term “CD3 receptor complex,” used herein, refers to a protein complex composed of four chains. In mammals, the complex contains a CD3γ (gamma) chain, a CD3δ (delta) chain, and two CD3ε (epsilon) chains. These chains associate with the T cell receptor (TCR) and the so-called ζ (zeta) chain to form the T cell receptor CD3 complex and to generate an activation signal in T lymphocytes. The CD3γ (gamma), CD3δ (delta), and CD3ε (epsilon) chains are highly related cell-surface proteins of the immunoglobulin superfamily containing a single extracellular immunoglobulin domain. The intracellular tails of the CD3 molecules contain a single conserved motif known as an immunoreceptor tyrosine-based activation motif or ITAM for short, which is essential for the signaling capacity of the TCR. The CD3 epsilon molecule is a polypeptide which in humans is encoded by the CD3E gene which resides on chromosome 11. The most preferred epitope of CD3 epsilon is comprised within amino acid residues 1-27 of the human CD3 epsilon extracellular domain.

The term “treatment” includes, e.g., ameliorating or reducing the severity of a disease, or shortening the length of the disease. Also, the term “treat,” as well as words related thereto, does not necessarily imply 100% or complete treatment. Rather, there are varying degrees of treatment of which one of ordinary skill in the art recognizes as having a potential benefit or therapeutic effect. In this respect, the methods of treating cancer of the present disclosure can provide any amount or any level of treatment. Furthermore, the treatment provided by the method of the present disclosure can include treatment of one or more conditions or symptoms or signs of the cancer being treated.

Therapeutic efficacy can be monitored by periodic assessment of treated patients. For repeated administrations over several days or longer, depending on the condition, the treatment can be repeated until a desired suppression of disease symptoms occurs. However, other dosage regimens may be useful and are within the scope of the disclosure.

“About” or “approximately,” when used in connection with a measurable numerical variable, refers to the indicated value of the variable and to all values of the variable that are within the experimental error of the indicated value (e.g. within the 95% confidence interval for the mean) or +10% of the indicated value, whichever is greater. Numeric ranges are inclusive of the numbers defining the range.

The present disclosure is not limited to the particular amino acid sequences described herein. In some embodiments, the disclosure provides amino acid sequences that are at least about 90% identical (e.g., at least 91%, at least 92%, at least 93%, at least 94%, at least 95%, at least 96%, at least 97%, at least 98%, at least 99%, or 100% identical) to any of the amino acid sequences identified herein. Amino acid or nucleic acid sequence “identity” can be determined by comparing a nucleic acid or amino acid sequence of interest to a reference nucleic acid or amino acid sequence. The percent identity is the number of nucleotides or amino acid residues that are the same (i.e., that are identical) as between the sequence of interest and the reference sequence divided by the length of the longest sequence (i.e., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL Omega, T-Coffee, and ALIGN (for alignment of nucleic acid and amino acid sequences), BLAST programs (e.g., BLAST 2.13, BL2SEQ, and later versions thereof) and FASTA programs (e.g., FASTA3x, FAS™, and S SEARCH) (for sequence alignment and sequence similarity searches). Sequence alignment algorithms also are disclosed in, for example, Altschul et al., J. Molecular Biol, 275 (3): 403-410 (1990), Beigert et al., Proc. Natl. Acad. Sci. USA, 106 (10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 27 (7): 951-960 (2005), Altschul et al., Nucleic Acids Res., 25 (17): 3389-3402 (1997), and Gusfield, Algorithms on Strings, Trees and Sequences, Cambridge University Press, Cambridge UK (1997)).

### Bispecific Antigen-Binding Molecules

As discussed above, the antigen-binding molecules provided herein are “bispecific” in that they bind to two different antigens or epitopes. In some embodiments, the bispecific antigen-binding molecule is an antigen-binding protein comprising at least a first binding domain that binds to human DLL3. In preferred embodiments, the bispecific antigen-binding molecule is an antigen-binding protein comprising at least a first binding domain that binds to human DLL3 and a second binding domain that binds to human CD3, a subunit of the T cell receptor complex on T cells as described above.

Delta-like ligand 3 (DLL3) is a non-canonical Notch ligand expressed primarily during embryonic development that functions during somitogenesis. DLL3 accumulates in the Golgi in normal tissues (Geffers et al, J Cell Biol., 178:465-476 (2007)). The human DLL3 protein comprises several extracellular domains: signal peptide, N-terminus, DSL, EGF1, EGF2, EGF3, EGF4, EGF5, EGF6, and a membrane proximal domain. Exemplary amino acid sequences of human DLL3 include, e.g., UniProt Q9NYJ7 and NCBI Reference Sequence: NP_058637.1.

The first binding domain may bind to any suitable region or epitope of human DLL3. In some embodiments, the first binding domain binds to an epitope of human DLL3 comprised within the EGF3 and EGF4 domains, such as an epitope within the amino acid sequence of SEQ ID NO: 1. For example, the first binding domain may bind to an epitope of human DLL3 within the EGF3 domain, which is comprised within the amino acid sequence of SEQ ID NO: 2. In other embodiments, the first binding domain of the bispecific antigen-binding molecule binds to an epitope of human DLL3 comprised within the EGF5 domains (SEQ ID NO: 3) and/or the EGF6 domain (SEQ ID NO: 4). One example of an agent targeting DLL3 is a bispecific T cell engaging antigen-binding polypeptide that binds DLL3 and CD3, such as a BiTE® molecule. BiTE® molecules are recombinant proteins comprised of two flexibly linked binding domains, each domain derived from antibodies. One binding domain of a BiTE® molecule is specific for a tumor-associated surface antigen (such as DLL3); the second binding domain is specific for CD3. By their design, BiTE® molecules are uniquely suited to transiently connect T cells with target cells and, at the same time, potently activate the inherent cytolytic potential of T cells against target cells. See e.g., WO 99/54440, WO 2005/040220, and WO 2008/119567.

In some embodiments, the bispecific antigen-binding protein may comprise two tandem scFv amino acid sequences (scFv)₂: one binding to human DLL3 and the other binding to human CD3. In this regard, for example, the amino acid sequence of a first scFv comprises a heavy chain variable region (VH) and a light chain variable region (VL) that binds to human DLL3, while the amino acid sequence of the second scFv comprises a VH and a VL that binds to human CD3. In some embodiments, the VH and VL are joined by a linker to form a single chain Fv (scFv). In some embodiments, the linker is a peptide linker comprising a sequence selected from any one of SEQ ID NOs: 5-13. In some embodiments, the linker is a GS liker, such as Gly-Gly-Gly-Gly-Ser (G4S, SEQ ID NO: 6), or polymers thereof, i.e, (Gly4Ser) x, where x is an integer of 1 or greater (e.g., 2 or 3) (e.g., SEQ ID NOs: 12 or 13).

In certain embodiments, the anti-DLL3 antigen-binding protein described herein comprises a first binding domain that binds to DLL3 (preferably human DLL3) and comprises the amino acid sequence of SEQ ID NO: 14, and a second binding domain that binds CD3 (preferably human CD3) and comprises the amino acid of SEQ ID NO: 15. In certain embodiments, the anti-DLL3 antigen-binding protein described herein comprises the amino acid sequence of SEQ ID NO: 16.

In various embodiments, the bispecific antigen-binding protein is tarlatamab (International Nonproprietary Names for Pharmaceutical Substances (INN): Proposed INN: List 123, WHO Drug Information 34 (2): 395-397 (2020)), also known as AMG 757, which is a half-life-extended BiTE® molecule developed for the treatment of SCLC. The activity of tarlatamab requires the simultaneous binding to both target cells (DLL3+ cells) and T cells. The pharmacological effect of tarlatamab is mediated by specific redirection of previously primed cytotoxic CD8+ or CD4+T lymphocytes to kill DLL3+ cells. Tarlatamab showed antitumor activity in SCLC patients in a Phase 1 study, and clinical evaluation is ongoing (see, e.g., ClinicalTrials.gov Identifier: NCT03319940 and Owonikoko et al., Journal of Clinical Oncology 39: 15_suppl, 8510-8510 (2021)). Tarlatamab is described in detail in, for example, WO 2017/021349 and WO 2021/092134.

In some embodiments, the bispecific antigen-binding molecule may be a heteromultimer. Exemplary heteromultimers include, but are not limited to, heterodimeric antibodies (used interchangeably herein with “hetero immunoglobulins” or “hetero Igs”), which are antibodies comprising two different light chains and two different heavy chains. In some embodiments, a heteromultimer encompassed by the disclosure comprises a first heterodimer that binds to human DLL3 and a second heterodimer that binds to human CD3.

In some embodiments, the first heterodimer comprises a heavy chain (HC) comprising the amino acid sequence of SEQ ID NO: 17 or SEQ ID NO: 21 and a light chain (LC) comprising the amino acid sequence of SEQ ID NO: 18 or SEQ ID NO: 22; and the second heterodimer comprises a heavy chain comprising the amino acid sequence of SEQ ID NO: 19 or SEQ ID NO: 24; and a light chain comprising the amino acid sequence of SEQ ID NO: 20. For example, the first heterodimer may comprise a heavy chain comprising the amino acid sequence of SEQ ID NO: 17 and a light chain comprising the amino acid sequence of SEQ ID NO: 18. Alternatively, the first heterodimer may comprise a heavy chain comprising the amino acid sequence of SEQ ID NO: 21 and a light chain comprising the amino acid sequence of SEQ ID NO: 22. In some embodiments, for example, the second heterodimer may comprise a heavy chain comprising the amino acid sequence of SEQ ID NO: 19 and a light chain comprising the amino acid sequence of SEQ ID NO: 20. In other embodiments, for example, the second heterodimer may comprise a heavy chain comprising the amino acid sequence of SEQ ID NO: 24 and a light chain comprising the amino acid sequence of SEQ ID NO: 20. In some embodiments, the first heterodimer may comprise a heavy chain comprising the amino acid sequence of SEQ ID NO: 23. In further embodiments, the second heterodimer may comprise a heavy chain comprising the amino acid sequence of SEQ ID NO: 25.

Thus, an exemplary heteromultimer provided herein may comprise a first heterodimer comprising a heavy chain amino acid sequence of SEQ ID NO: 17 and a light chain amino acid sequence of SEQ ID NO: 18; and a second heterodimer comprising a heavy chain amino acid sequence of SEQ ID NO: 19 and a light chain amino acid sequence of SEQ ID NO: 20. In other embodiments, an exemplary heteromultimer provided herein may comprise a first heterodimer comprising a heavy chain amino acid sequence of SEQ ID NO: 17 and a light chain amino acid sequence of SEQ ID NO: 18; and a second heterodimer comprising a heavy chain amino acid sequence of SEQ ID NO: 24 and a light chain amino acid sequence of SEQ ID NO: 20. In some embodiments, an exemplary heteromultimer provided herein comprises a first heterodimer comprising a heavy chain amino acid sequence of SEQ ID NO: 21 and a light chain amino acid sequence of SEQ ID NO: 22; and a second heterodimer comprising a heavy chain amino acid sequence of SEQ ID NO: 19 and a light chain amino acid sequence of SEQ ID NO: 20. In other embodiments, an exemplary heteromultimer provided herein comprises a first heterodimer comprising a heavy chain amino acid sequence of SEQ ID NO: 21 and a light chain amino acid sequence of SEQ ID NO: 22; and a second heterodimer comprising a heavy chain amino acid sequence of SEQ ID NO: 24 and a light chain amino acid sequence of SEQ ID NO: 20.

Other exemplary antigen-binding binding proteins that bind to DLL3 and CD3 encompassed by the present disclosure include multi-specific binding proteins described in, e.g., Hipp et al., Clin Cancer Res 2020; 26:5258-68; WO 2019/234220; and WO 2020/069028. In certain embodiments, the bispecific antigen-binding protein is a protein that comprises (a) a first domain which is a single chain variable fragment (scFv) that specifically binds to a human CD3; (b) a second domain which is a single domain antibody that specifically binds to a human serum albumin protein; and (c) a third domain which is a single domain antibody that specifically binds to a DLL3 protein. In certain embodiments, the bispecific antigen-binding protein comprises or consists of the amino acid sequence of SEQ ID NO: 26 or 27. In certain embodiments, the bispecific antigen-binding protein is a protein comprising (a) a first antigen binding domain that specifically binds to human DLL3; (b) a second antigen binding domain that specifically binds to human CD3, and (c) a first and a second Fc domain, wherein the first Fc domain is covalently linked to the first antigen binding domain, and the second Fc domain is covalently linked to the second antigen binding domain. In certain embodiments, the first binding domain specifically binds to the membrane proximal region of the human DLL3 (e.g., SEQ ID NO: 28). In certain embodiments, the first binding domain comprises from its N- to C-terminus a first light chain variable domain, a first light chain constant domain, a first peptide linker, a first heavy chain variable domain and a first heavy chain constant CH1 domain; and the second binding domain comprises from its N- to C-terminus a second light chain variable domain, a second light chain constant domain, a second peptide linker, a second heavy chain variable domain and a second heavy chain constant CH1 domain.

In some embodiments, the antigen-binding molecule comprises an immunoglobulin constant region. The term “constant region” as used herein refers to all domains of an antibody other than the variable region. The constant region is not involved directly in binding of an antigen, but exhibits various effector functions. As described above, antibodies are divided into particular isotypes (IgA, IgD, IgE, IgG, and IgM) and subtypes (IgG1, IgG2, IgG3, IgG4, IgA1 IgA2) depending on the amino acid sequence of the constant region of their heavy chains. The light chain constant region can be, for example, a kappa- or lambda-type light chain constant region, e.g., a human kappa- or lambda-type light chain constant region, which are found in all five antibody isotypes. In some embodiments, an antigen-binding protein encompassed by the present disclosure is of the IgG1 or IgG4 isotype.

### DLL3 Expression

The disclosure provides a method of treating DLL3-expressing cancer (e.g., small cell lung cancer (SCLC)) in a subject. For any of the methods of treating SCLC described herein, the SCLC cancer cells desirably express DLL3 on the cell surface. In other embodiments, the SCLC cancer cells express DLL3 on the cell surface and in the cytoplasm. In some aspects, cell surface expression and intercellular expression of DLL3 protein may be determined by immunohistochemistry (IHC) or positron emission tomography (PET). Any suitable IHC assay for determining DLL3 protein expression may be used in connection with the present disclosure. Desirably, the IHC assay is approved by a regulatory agency, such as the U.S. Food and Drug Association (FDA) or an agency that has CE-IVD registration authority. DLL3-specific IHC assays, and components thereof, are known in the art and commercially available from a variety of sources. For example, DLL3 expression in cancer or tumor cells may be detected using the VENTANA® DLL3 (SP347) Assay (Roche Diagnostics, GmbH, Mannheim, Germany). Other anti-DLL3 antibodies that may be used to detect DLL3 expression in an IHC assay include, but are not limited to, NBP2-24669 (Novus Biological, Littleton, CO); PA5-26336 (Thermo Fisher Scientific, Waltham, MA); and ab229902 (Abcam, Cambridge, MA).

In other embodiments, DLL3 gene expression in SCLCs may be determined. Methods for detecting and quantifying gene expression (e.g., mRNA levels) are known in the art and can be used in the context of the present disclosure. Such methods include, for example, flow cytometry-based methods polymerase chain reaction (PCR) analysis, sequencing analysis (e.g., RNA sequencing), electrophoretic analysis, restriction fragment length polymorphism (RFLP) analysis, Northern blot analysis, quantitative PCR, reverse-transcriptase-PCR analysis (RT-PCR), and the like.

In some embodiments, at least 5% (e.g., 5%, 10%, or 20%) of the cells of a cancer may be positive for DLL3 as determined by IHC. The amount of DLL3-positive SCLC cells in a sample obtained from a subject may be expressed in terms of the tumor proportion score (TPS). The “tumor proportion score” is the percentage of viable cancer or tumor cells showing partial or complete membrane and cytoplasmic staining for DLL3. In some embodiments, at least 25% (e.g., 30%, 35%, 40%, or 45%) of the SCLC cells in a subject, or in an SCLC sample obtained from a subject, express DLL3 (25% TPS). In certain embodiments, at least 50% (e.g., 55%, 60%, 65%, 70%, 80%, 90%, 99%, or 100%) of the SCLC cells in a subject, or in an SCLC sample obtained from a subject, express DLL3 (50% TPS). For example, at least 75% (e.g., 85%, 95%, or 100%) of the SCLC cells in a subject, or in an SCLC sample obtained from a subject, expresses DLL3 (75% TPS). Viable cancer or tumor cells refer to cancer or tumor cells with normal morphology and the intact nuclei (e.g., absence of nuclear blebbing) and are a qualitative assessment routinely performed by a person of ordinary skill in the art, e.g., a pathologist. In certain embodiments, a viable cancer or tumor cell is determined by a pathologist.

For example, in embodiments where the VENTANA® DLL3 (SP347) Assay (Roche Diagnostics, GmbH, Mannheim, Germany) is used, the following DLL3 scoring algorithm may be employed:
Positive Status DLL3 (SP347) at 25% cut off: ≥25% of viable tumor cells demonstrate moderate to strong membrane and/or cytoplasmic staining using any objective.
Positive Status DLL3 (SP347) at 75% cut off: ≥75% of viable tumor cells demonstrate moderate to strong membrane and/or cytoplasmic staining using any objective.

Certain considerations of the above scoring algorithm include, for example, (i) no restriction on magnification used; (ii) at least 100 viable tumor cells with well-preserved morphology are required; and (iii) partial/incomplete cellular (cyto or membranous) staining is taken into account and the highest intensity is considered in evaluation.

The level of DLL3 expression by IHC may also be determined by giving a tumor sample an IHC score based on DLL3-positive signal intensity. In some embodiments, tumor scoring is based on the methodology described in Huang et al, Arch Pathol Lab Med, 143 (11): 1373-137 (2019). For example, to evaluate DLL3 staining, combined membrane and cytoplasmic H-scores may be collected at any magnification. H-score may be calculated using the following formula: 1× (percentage of cells staining at 1+ intensity)+2×(percentage of cells staining at 2+ intensity)+3×(percentage of cells staining at 3+ intensity) and is typically in the range of 0-300. Surprisingly, inclusion of cytoplasmic DLL3 staining with membrane DLL3 staining does not diminish the predictive/correlative value of DLL3 expression with respect to therapeutic outcomes, and in fact results in a more efficient analysis. Negative or weak staining may be confirmed at a minimum of 20× magnification. DLL3 positivity may be defined as ≥1% stained tumor cells. Hematoxylin and Eosin (H&E) staining may be performed using routine methods, and H&E-stained slides may be used to evaluate for tumor content and assessment of tissue quality. In some embodiments, a minimum of 100 viable tumor cells (e.g., 120, 130, 140, 150, 200, 300, 400, 500 or more viable tumor cells) are used for DLL3 evaluation. Tumor tissues may be scored for specific percentage tumor cell staining, as discussed above. Tumor tissues may also be scored for overall intensity of tumor cell DLL3 staining on a scale of 0 to 3 in increments of 1. For example, strong cytoplasmic and/or membranous DLL3 staining of tumor cells may be scored as “3” or “3+,” moderate staining may be scored as “2” or “2+,” and weak staining may be scored as “1” or “1+.” The absence of any DLL3-positive staining may be given a score of “0.” In some embodiments, an IHC intensity score of 2, 2+, 3, or 3+ (e.g., 2+. 3+, or 2+ and 3+) indicates a high level of DLL3 expression or DLL3 overexpression. In other embodiments, 3+ staining of tumor cells by IHC indicates a high level of DLL3 expression or DLL3 overexpression. In some embodiments, an IHC intensity score (e.g., 1+, 2+, or 3+) is determined by a pathologist.

Thus, the present disclosure also provides a method of treating SCLC in a subject, which comprises administering to the subject a bispecific antigen-binding molecule comprising at least a first binding domain that binds to human DLL3, wherein at least 25% (e.g., 30%, 35%, 40%, 45%) of the SCLC cells express DLL3 as determined by, e.g., an IHC assay. In certain embodiments, at least 50% (e.g., 55%, 60%, 65%, 70%, 80%, 90%, 99%, or 100%) of the SCLC cells express DLL3. For example, at least 75% (e.g., 85%, 95%, or 100%) of the SCLC cells expresses DLL3. In certain embodiments, the present disclosure provides a method of treating SCLC in a subject, which comprises administering to the subject a bispecific antigen-binding molecule comprising at least a first binding domain that binds to human DLL3, wherein the SCLC has a DLL3 expression level in which at least 25% (e.g., 30%, 40%, 50%, 60%, 70% or more) of the SCLC cells express DLL3 at an intensity equal to or greater than 2+ (e.g., 2+, 3+, or 2+ and 3+) as determined by an IHC assay. For example, in some embodiments, the SCLC has a DLL3 expression level in which at least 75% (e.g., 80%, 85%, 90%, 95%, 99%, or 100%) of the SCLC cells express DLL3 at an intensity equal to or greater than 2+ (e.g., 2+, 3+, or 2+ and 3+) as determined by an IHC assay.

In accordance with the inventive method, the intensity of the IHC signal(s) detected in a sample may be compared to a predetermined reference value, such as a predetermined threshold value. The term “predetermined reference value” refers to an assay value, or defined amount of analyte, that is used to assess an assay result. For example, a predetermined reference value may be a DLL3-positive signal corresponding to a known quantity of DLL3 protein. The terms “predetermined threshold value” and “predetermined cut-off value” are used interchangeably herein to refer to an assay value, or defined amount of an analyte, that is used to assess DLL3 expression in a tumor sample from a subject by comparing the assay results against the predetermined threshold or cut-off value. While the present disclosure may provide exemplary predetermined threshold levels, it is well-known that cut-off values may vary depending on the nature of the assay (e.g., antibodies employed, etc.). It further is well within the ordinary skill in the art to adapt the disclosure herein for other samples to obtain assay-specific reference or cutoff values for such assays based on this disclosure. Without being limited by theory, the use of paired, nested cut-offs (such as 25% TPS or 75% TPS) for identifying DLL3-expressing cancers ensures that moderate and high expressing tumor cells are included while also allowing analysis of strong DLL3 expression.

### Subjects and Samples

In various instances of the disclosed methods, the subject is a human subject. In exemplary instances, the human subject has Small Cell Lung Cancer (SCLC), optionally, a histologically or cytologically confirmed SCLC. In various aspects, the human is male or female and/or greater than or equal to 18 years of age with a SCLC. In exemplary aspects, the human subject has been treated with a platinum-based chemotherapy. In exemplary aspects, the human subject has relapsed refractory (RR) SCLC, optionally, which progressed or recurred following at least one platinum-based chemotherapy with or without a PD-L1 inhibitor. In exemplary aspects, the human subject has extensive-stage small cell lung cancer (ES-SCLC), optionally, histologically or cytologically confirmed ES-SCLC. In exemplary aspects, the human subject has limited stage SCLC, optionally, which progressed or recurred following at least one platinum-based chemotherapy. In exemplary aspects, the human subject has ES-SCLC and has received no prior systemic treatment for ES-SCLC. In exemplary instances, the human subject has an Eastern Cooperative Oncology Group (ECOG) performance status of 0-1 (Oken et al., Am J Clin Oncol, 5:649-655 (1982). In various aspects, the human subject has one or more brain metastases that have been treated. In various aspects, the platinum-based chemotherapy comprises carboplatin or cisplatin or platinum-irinotecan.

DLL3 expression analysis as described herein desirably is performed on a sample of tumor or cancer cells obtained from a subject diagnosed with cancer. The terms “sample” or “biological sample” as used herein, refer to a sample of biological fluid, tissue, or cells, in a healthy and/or pathological state obtained from a subject. A variety of cell types, tissue, or bodily fluid may be utilized as a sample. Such cell types, tissues, and fluid may include sections of tissues such as biopsy and autopsy samples, frozen sections taken for histologic purposes, blood (such as whole blood), plasma, serum, red blood cells, platelets, bronchial lavage fluid, interstitial fluid, cerebral spinal fluid, etc. A tissue or cell sample may be provided by removing cells from a human, but can also be accomplished by using previously isolated cells (e.g., isolated by another person, at another time, and/or for another purpose). Archival tissues, such as those having treatment or outcome history, may also be used. In some aspects, the sample comprises formalin fixed paraffin embedded (FFPE) cytology or tissue specimens from the subject. Exemplary cytology or tissue specimens include core need biopsy (CNB), fine needle aspiration (FNA), endobronchial ultrasound scan and biopsy (EBUS). Cytology or tissue specimens are eligible for testing as long as they are fixed in formalin and prepared into FFPE (e.g., FFPE of a freshly core needle biopsy) specimens.

In exemplary aspects, the cancer is a histologically or cytologically confirmed SCLC. Optionally, the SCLC is measurable by modified Response Criteria in Solid Tumors (RECIST) 1.1, wherein measurable lesions include (a) non-nodal lesions with clear borders that can be measured accurately and serially in one dimension in the axial plane (longest diameter ≥10 mm measured by magnetic resonance imaging/computed tomography (MRI/CT) with scan slice thickness ≤5 mm) and/or (b) nodal lesions with the longest diameter perpendicular to the long axis (short axis) ≥15 mm on MRI/CT, and/or exclude simple cysts, pleural/pericardial effusions and ascites.

The cancer may be a neuroendocrine cancer other than SCLC. Neuroendocrine cancers or neoplasms (NECs or NENs) are a relatively rare and heterogeneous tumor type, comprising ~2% of all malignancies, with a prevalence of <200,000 in the United States (Oronsky et al., Neoplasia, 19 (12): 991-1002 (2017)). The term “neuroendocrine” is applied to widely dispersed cells with properties similar to neural cells, such as the presence of dense core granules (DCGs4) that are similar to DCGs present in serotonergic neurons, which store monoamines, and “endocrine” properties, such as the synthesis and secretion of these monoamines. The neuroendocrine (NE) system includes endocrine glands, such as the pituitary, the parathyroids, and the NE adrenal, as well as endocrine islet tissue embedded within glandular tissue (thyroid or pancreatic) and scattered cells in the exocrine parenchyma, such as endocrine cells of the digestive and respiratory tracts, which belong to what is known as the diffuse endocrine system. Most neuroendocrine tumors occur in the lungs, appendix, small intestine, rectum and pancreas. Neuroendocrine cancers include, but are not limited to, small cell lung cancer (SCLC), neuroendocrine prostate cancer (NEPC), and neuroblastoma. In some embodiments, the tumor or cancer is lung cancer such as small cell lung cancer (SCLC) or non-small cell lung cancer (NSCLC), glioma, glioblastoma, melanoma, prostate cancer such as neuroendocrine prostate cancer, neuroendocrine pancreatic cancer, hepatoblastoma, large cell pulmonary neuroendocrine cancer, pancreatic neuroendocrine cancer, bladder neuroendocrine cancer, gastric neuroendocrine cancer, adrenal exocrine tumors, Merkel cell carcinoma, neuroblastoma, head and neck carcinoid or neuroendocrine cancer, head and neck paraganglioma, or cervical small cell neuroendocrine cancer. In some embodiments, the tumor or cancer is prostate cancer (e.g., neuroendocrine prostate cancer) or lung cancer (e.g., small cell lung cancer).

### Dosing Regimens

Disclosed herein are methods of treating a cancer (e.g., SCLC) in which least 25% of the cancer cells express DLL3 or in which at least 25% of the cancer cells express DLL3 at an intensity of equal to or greater than 2+ comprising administering to a subject in need thereof a bispecific antigen-binding molecule, such as the anti-DLL3 antigen-binding molecules described herein. In some aspects, the anti-DLL3 antigen-binding molecule is administered to a subject at a dose of from about 10 mg to about 100 mg, from about 30 mg to about 100 mg, about 10 mg, or about 100 mg, once every two weeks. In other embodiments, the anti-DLL3 antigen-binding molecule is administered to a subject at a dose of from about 10 mg to about 100 mg, from about 30 mg to about 100 mg, about 10 mg, or about 100 mg, twice every three weeks. In other embodiments, the anti-DLL3 antigen-binding molecule is administered to a subject at a dose of from about 20 mg to about 200 mg, from about 30 mg to about 100 mg, about 20 mg, about 60 mg, or about 200 mg once every three weeks. As discussed above, in certain embodiments, the DLL3-positive cancer is small cell lung cancer (SCLC). In certain embodiments, the SCLC is limited stage SCLC, relapsed/refractory SCLC (RR SCLC) or extensive disease SCLC (ED SCLC). In certain embodiments, the subject is a human having SCLC, e.g., RR SCLC or ED SCLC.

An exemplary dosing regimen comprises administering the anti-DLL3 antigen-binding molecule to a subject once a week for three weeks (i.e., weeks 1, 2, and 3), followed by administering the anti-DLL3 antigen-binding molecule to the subject once every two weeks. Another exemplary dosing regimen comprises administering the anti-DLL3 antigen-binding molecule to a subject once a week for two weeks (i.e., weeks 1 and 2), followed by administering the anti-DLL3 antigen-binding molecule to the subject once every three weeks. The anti-DLL3 antigen-binding molecule may be administered to the subject at a dose of about 10 mg to about 100 mg, about 30 mg to about 100 mg, about 10 mg, or about 100 mg.

In certain embodiments, the anti-DLL3 antigen-binding molecule is administered on day 1 and day 15 of a 28-day cycle. In other embodiments, the anti-DLL3 antigen-binding molecule is administered on day 1 of a 21-day cycle, or on day 1 and day 8 of a 21-day cycle.

The anti-DLL3 antigen-binding molecule can be administered by any suitable means, including parenteral, subcutaneous, intraperitoneal, intrapulmonary, intranasal, and/or intralesional administration. Parenteral administration includes intramuscular, intravenous, intraarterial, intraperitoneal, or subcutaneous administration. In some embodiments, the anti-DLL3 antigen-binding molecule is administered by intravenous (IV) infusion, such as a short IV infusion (approximately 60 minutes), once every two weeks, once every three weeks, or twice every three weeks.

Subjects can have an increased risk for cytokine release syndrome (CRS) during initiation of treatment with a bispecific antigen-binding molecule (e.g., an anti-DLL3 antigen-binding molecule such as those described herein) due to its mechanism of action, and a step dosing approach may be implemented. Thus, in certain embodiments, a bispecific antigen-binding molecule (e.g., an anti-DLL3 antigen-binding molecule) is administered using a step dosing approach during initiation of treatment of the agent (e.g., the first cycle of treatment) before the anti-DLL3 antigen-binding molecule is administered according to the once every two weeks, twice every three weeks, or once every three weeks regimen described above. For example, an anti-DLL3 antigen-binding molecule may be administered in a 21-day or a 28-day cycle, such as those described above, during initiation of the treatment (cycle 1) according to the following one step dose regimen: a first step dose or run-in dose on day 1, a step dose that equals to target dose on day 8, and a target dose on day 15. Exemplary run-in doses of an anti-DLL3 antigen-binding molecule include 1 mg. Exemplary target doses of an anti-DLL3 antigen-binding molecule include a dose in the range of from 10 mg to 100 mg (e.g., 10 mg or 100 mg) or a dose in the range of from 20 mg to 200 mg (e.g., 20 mg, 60 mg or 200 mg).

### Combination Therapies

In some embodiments of the methods disclosed herein, the above-described bispecific antigen-binding molecule (e.g., an anti-DLL3 antigen-binding protein) may be administered alone (i.e., as a “monotherapy”) to treat a cancer (e.g., SCLC) in which least 25% of the cancer cells express DLL3 or in which at least 25% of the cancer cells express DLL3 at an intensity of equal to or greater than 2+, or in combination with at least one additional therapeutic agent to achieve a desired biological effect in a subject (i.e., as part of a “combination therapy”). In various embodiments disclosed herein, “combination therapy” or “in combination with” refers to administration of one treatment modality (e.g., an anti-DLL3 antigen-binding protein) in addition to another treatment modality (e.g., an anti-PD-L1 antibody and optionally one or more chemotherapeutic agents) to a subject (e.g., a human) having DLL3-expressing cancer. In combination therapies wherein an anti-DLL3 antigen-binding molecule and an anti-PD-L1 antibody are involved (as described below), one treatment modality can be administered before, during, or after administration of the other treatment modality to the subject. However, such combination therapy does not include situations wherein 28 or more days have elapsed between the end of administration of one treatment modality and the beginning of administration of another treatment modality.

In some embodiments, the anti-DLL3 antigen-binding molecule is administered in combination with one or more chemotherapeutic agents. “Chemotherapeutic agents,” also referred to as antineoplastic agents, include compounds useful for the treatment of cancer. Chemotherapeutic agents can be classified according to their mechanism of action and can be further divided into subgroups within each class. Exemplary classes of chemotherapeutic agents include alkylating agents, antimetabolites, topoisomerase inhibitors, anti-tumor antibiotics, mitotic inhibitors, and protein kinase inhibitors. Alkylating agents include subgroups such as oxazaphosphorines, nitrogen mustards, imidazotetrazines, nitrosoureas, alkyl sulfonate, hydrazines, and platinum-based agents. Platinum-based agents include cisplatin, carboplatin, and oxaliplatin. Topoisomerase inhibitors include topoisomerase I inhibitors and topoisomerase II inhibitors. Mitotic inhibitors include vinca alkaloids, taxanes, and nontaxane microtubule inhibitors. Anti-tumor antibiotics include bleomycin, actinomycin D (dactinomycin), and mitomycin.

In certain embodiments, the chemotherapeutic agent that can be used in the method disclosed herein is an alkylating agent. In exemplary embodiments, the alkylating agent may be a platinum-based agent such as cisplatin, carboplatin, or oxaliplatin. In certain embodiments, the alkylating agent is lurbinectedin (ZEPZELCA™). In other embodiments, the chemotherapeutic agent may be a topoisomerase inhibitor such as a topoisomerase II inhibitor (e.g., etoposide). In certain embodiments, the chemotherapeutic agent that can be used in the methods disclosed herein includes a platinum-based agent (cisplatin, carboplatin, or oxaliplatin), a topoisomerase II inhibitor (etoposide) or a combination of a platinum-based agent and a topoisomerase II inhibitor.

In some aspects of the disclosed method, the anti-DLL3 antigen-binding molecule is administered in combination with an antagonist of the PD-1/PD-L1 signaling pathway. Programmed Cell Death protein 1 (PD-1), also known as CD279, SLEB2, and hSLE1, is a transmembrane protein expressed on activated T, natural killer (NK) and B lymphocytes, macrophages, dendritic cells (DCs) and monocytes. Notably, PD-1 is highly expressed on tumor-specific T cells (Han et al., Am J Cancer Res 10 (3): 727-742 (2020)). PD-1 binds to B7 protein family members, PD-1 Ligand 1 (PD-L1; also referred to as CD279 and B7-H1) and PD-1 Ligand 2 (also known as PD-L2, CD273, and B7-DC). PD-L1 is constitutively expressed on T and B cells, macrophages and dendritic cells, whereas PD-L2 expression is typically restricted to activated DC and macrophages (Xing et al., Oncoimmunology 7 (3): e1356144 (2017) (doi: 10.1080/2162402X.2017.1356144)). PD-1 inhibits both adaptive and innate immune responses. The PD-1/PD-L1 axis is involved in the suppression of T cell immune responses in cancer. Antagonists of this pathway have been clinically validated across a number of solid tumor indications. PD-1 inhibitors, e.g., nivolumab, pembrolizumab, and cemiplimab, and PD-L1 inhibitors, e.g., atezolizumab, avelumab, and durvalumab, target the PD-1/PD-L1 pathway, and each has been approved by the U.S. Food and Drug Administration (FDA) for the treatment of various cancers. In various embodiments, agents targeting PD-L1 (e.g., PD-L1 blocking agent) can be used in methods disclosed herein to treat DLL3-expressing cancers. Exemplary agents targeting PD-L1 include anti-PD-L1 antibodies such as atezolizumab, avelumab, and durvalumab.

In certain embodiments, the anti-PD-L1 antibody is atezolizumab (International Nonproprietary Name for Pharmaceutical Substances (INN), WHO Drug Information, Vol. 29, No. 3, 2015, Recommended INN: List 74). Atezolizumab is a humanized PD-L1 blocking antibody. It is an immunoglobulin G1-kappa, anti-[Homo sapiens CD274 (programmed death ligand 1, PDL1, PD-L1, B7 homolog 1, B7H1)], humanized monoclonal antibody; gamma1 heavy chain (1-448) [humanized VH (Homo sapiens IGHV3-23*04 (86.70%)-(IGHD)-IGHJ4*01) [8.8.11] (1-118)-Homo sapiens IGHG1*03 (CH1 R120>K (215) (119-216), hinge (217-231), CH2 N84.4>A (298) (232-341), CH3 (342-446), CHS (447-448)) (119-448)], (221-214′)-disulfide with kappa light chain (1′-214′) [humanized V-KAPPA (Homo sapiens IGKV1-5*01 (87.90%)-IGKJ1*01) [6.3.9] (1′-107′)-Homo sapiens IGKC*01 (108′-214′)]; dimer (227-227″: 230-230″)-bisdisulfide. Atezolizumab is available commercially, e.g., it is marketed as TECENTRIQ®.

In certain embodiments, the anti-PD-L1 antibody is avelumab (International Nonproprietary Name for Pharmaceutical Substances (INN), WHO Drug Information Vol. 30, No. 1, 2016, Recommended INN: List 75). Averlumab is a PD-L1 blocking monoclonal antibody produced in CHO cells. It is an immunoglobulin G1-lambda1, anti-[Homo sapiens CD274 (programmed death ligand 1, PDL1, PD-L1, B7 homolog 1, B7H1)], Homo sapiens monoclonal antibody; gamma1 heavy chain (1-450) [Homo sapiens VH (IGHV3-23*01 (90.80%)-(IGHD)-IGHJ4*01) [8.8.13] (1-120)-IGHG1*01, Gm17,1 (CH1 (121-218), hinge (219-233), CH2 (234-343), CH3 (344-448), CHS (449-450) (121-450)], (223-215′)-disulfide with lambda1 light chain (1′-216′) [Homo sapiens V-LAMBDA (IGLV2-14*01 (99.00%)-IGLJ1*01) [9.3.10] (1′-110′)-IGLC1*02 (111′-216′)]; dimer (229-229″: 232-232″)-bisdisulfide. Avelumab is commercially available, e.g., it is marketed as BAVENCIO®.

In certain embodiments, the anti-PD-L1 antibody is durvalumab (International Nonproprietary Name for Pharmaceutical Substances (INN), WHO Drug Information, Vol. 29, No. 3, 2015, Recommended INN: List 74). Durvalumab is a PD-L1 blocking monoclonal antibody produced in CHO cells. It is an immunoglobulin G1-kappa, anti-[Homo sapiens CD274 (programmed death ligand 1, PDL1, PD-L1, B7 homolog 1, B7H1)], Homo sapiens monoclonal antibody; gamma1 heavy chain (1-451) [Homo sapiens VH (IGHV3-7*01 (99.00%)-(IGHD)-IGHJ4*01) [8.8.14] (1-121)-IGHG1*03 (CH1 (122-219), hinge (220-234), CH2 (235-344) L1.3>F (238), L1.2>E (239), P116>S (335), CH3 (345-449), CHS (450-451)) (122-451)], (224-215′)-disulfide with kappa light chain (1′-215′) [Homo sapiens V-KAPPA (IGKV3-20*01 (96.90%)-IGKJ1*01) [7.3.9] (1′-108′)-IGKC*01 (109′-215′)]; dimer (230-230″: 233-233″)-bisdisulfide. Durvalumab is commercially available, e.g., it is marketed as IMFINZI®.

In one aspect, disclosed herein is a method of treating a DLL3-expressing cancer (e.g., SCLC) in which at least 25% of the cancer cells express DLL3 or in which at least 25% of the cancer cells express DLL3 at an intensity of equal to or greater than 2+ comprising administering to a subject in need thereof an anti-DLL3 antigen-binding molecule, an anti-PD-L1 antibody, and optionally one or more chemotherapeutic agents, wherein the anti-DLL3 antigen-binding molecule is administered at a dose of from about 10 mg to about 100 mg once every two weeks. In certain embodiments, the anti-DLL3 antigen-binding molecule is administered at a dose of about 10 mg, about 30 mg, about 50 mg, or about 100 mg once every two weeks. In certain embodiments, the anti-DLL3 antigen-binding molecule is administered on day 1 and day 15 of a 28-day cycle.

In one aspect, disclosed herein is a method of treating DLL3-expressing cancer (e.g., SCLC) in which at least 25% of the cancer cells express DLL3 or in which at least 25% of the cancer cells express DLL3 at an intensity of equal to or greater than 2+ comprising administering to a subject in need thereof an anti-DLL3 antigen-binding molecule, an anti-PD-L1 antibody, and optionally one or more chemotherapeutic agents, wherein the anti-DLL3 antigen-binding molecule is administered at a dose of from about 10 mg to about 100 mg twice every three weeks, or at a dose of from about 20 mg to about 200 mg once every three weeks. In certain embodiments, the anti-DLL3 antigen-binding molecule is administered at a dose of about 10 mg, about 30 mg, about 50 mg, or about 100 mg twice every three weeks, e.g., on day 1 and day 8 of a 21-day cycle. In certain embodiments, the anti-DLL3 antigen-binding molecule is administered at a dose of from about 20 mg to about 100 mg (e.g., about 20 mg, about 60 mg, or about 100 mg) once every three weeks, e.g., on day 1 a 21-day cycle. In certain embodiments, the anti-DLL3 antigen-binding molecule is administered at a dose of from about 100 mg to about 200 mg (e.g., about 120 mg or about 200 mg) once every three weeks, e.g., on day 1 a 21-day cycle.

In various embodiments wherein an anti-DLL3 antigen-binding molecule is administered together with an anti-PD-L1 antibody and optionally one or more chemotherapeutic agents, the anti-DLL3 antigen-binding molecule can be administered according to a step dose regimen during initiation of treatment (e.g., cycle 1) to minimize potential adverse effects (e.g., CRS) associated with the anti-DLL3 antigen-binding molecule, as described herein. Thus, in certain embodiments, the anti-DLL3 antigen-binding molecule is administered in a 21-day cycle according to the following regimen in cycle 1 of treatment: a first dose of 0 mg or about 1 mg on day 1, a second dose of from about 1 mg to about 100 mg on day 8, and a third dose of from about 10 mg to about 200 mg on day 15. In certain embodiments, the anti-DLL3 antigen-binding molecule is administered according to the following regimen in cycle 1: a first dose of about 1 mg on day 1, a second dose of from about 10 mg to about 100 mg on day 8, and a third dose of from about 10 mg to about 100 mg on day 15. In certain embodiments, the anti-DLL3 antigen-binding molecule is administered according to the following regimen in cycle 1: a first dose of about 1 mg on day 1, a second dose of from about 10 mg to about 100 mg on day 8, and a third dose of from about 20 mg to about 200 mg on day 15. Each of the cycle 1 regimens can be used prior to the once every two weeks, twice every three weeks, or once every three weeks regimen for the anti-DLL3 antigen-binding molecule described above.

In certain embodiments, disclosed herein is a method of treating DLL3-expressing cancer (e.g., SCLC) in which at least 25% of the cancer cells express DLL3 or in which at least 25% of the cancer cells express DLL3 at an intensity of equal to or greater than 2+ comprising administering to a subject in need thereof an anti-DLL3 antigen-binding molecule, an anti-PD-L1 antibody, and optionally one or more chemotherapeutic agents, wherein the anti-DLL3 antigen-binding molecule is administered according to the following regimen: a) cycle 1 (21 days): a first dose of about 1 mg on day one, a second dose on day 8, a third dose on day 15, b) cycles 2 and cycle 3 (21 days each cycle): a fourth dose on day 1 and a fifth dose on day 8 of each cycle, and c) one or more subsequent doses once every two weeks in a 28-day cycle starting in cycle 4 and thereafter, wherein the second, third, fourth, fifth and the one or more subsequent doses are the same and are each from about 10 mg to about 100 mg (e.g., about 10 mg, about 30 mg, or about 100 mg).

In certain embodiments, disclosed herein is a method of treating DLL3-expressing cancer (e.g., SCLC) in which at least 25% of the cancer cells express DLL3 or in which at least 25% of the cancer cells express DLL3 at an intensity of equal to or greater than 2+ comprising administering to a subject in need thereof an anti-DLL3 antigen-binding molecule, an anti-PD-L1 antibody, and optionally one or more chemotherapeutic agents, wherein the anti-DLL3 antigen-binding molecule is administered in a 21-day cycle according to the following regimen: a) cycle 1: a first dose of about 1 mg on day one, a second dose on day 8, a third dose on day 15, b) cycles 2 and cycle 3: a fourth dose on day 1 and a fifth dose on day 8 of each cycle, and c) one or more subsequent doses once every three weeks starting in cycle 4 and thereafter, wherein the second, third, fourth, and fifth doses are the same and are each from about 10 mg to about 100 mg (e.g., about 10 mg, about 30 mg, or about 100 mg), and wherein the one or more subsequent doses are the same and are each from about 20 mg to about 200 mg (e.g., about 20 mg, about 60 mg, or about 200 mg).

In certain embodiments, disclosed herein is a method of treating DLL3-positive cancer (e.g., SCLC) in which at least 25% of the cancer cells express DLL3 or in which at least 25% of the cancer cells express DLL3 at an intensity of equal to or greater than 2+ comprising administering to a subject in need thereof an anti-DLL3 antigen-binding molecule, an anti-PD-L1 antibody, and optionally one or more chemotherapeutic agents, wherein the anti-DLL3 antigen-binding molecule is administered in a 21-day cycle according to the following regimen: a) cycle 1: a first dose of about 1 mg on day one, a second dose of from about 10 mg to about 100 mg on day 8, a third dose on day 15, b) cycles 2 and cycle 3: a fourth dose on day 1 of each cycle, and c) one or more subsequent doses once every three weeks starting in cycle 4 and thereafter, wherein the third, fourth, and the one or more subsequent doses are the same and are each from about 20 mg to about 200 mg (e.g., about 20 mg, about 60 mg, or about 200 mg).

In various embodiments wherein the anti-DLL3 antigen-binding molecule is administered together with the anti-PD-L1 antibody and optionally one or more chemotherapeutic agents, the anti-PD-L1 antibody is a PD-L1 blocking antibody. Examples of such anti-PD-L1 antibody include atezolizumab, durvalumab, and avelumab. In certain embodiments, the anti-PD-L1 antibody is atezolizumab or durvalumab. In certain embodiments, the anti-PD-L1 antibody is durvalumab. When used in methods disclosed herein, the dose and regimen of the anti-PD-L1 antibodies are the same as approved by regulatory agencies (e.g., the FDA). For example, atezolizumab can be administered at a dose of about 840 mg every 2 weeks, or about 1200 mg every 3 weeks, or about 1680 mg every 4 weeks. For example, durvalumab can be administered at a dose of about 10 mg/kg every 2 weeks, or about 1500 mg every three weeks, or about 1500 mg every four weeks.

In various embodiments wherein the anti-DLL3 antigen-binding molecule is administered together with the anti-PD-L1 antibody and optionally one or more chemotherapeutic agents, the one or more chemotherapeutic agents include an alkylating agent, a topoisomerase inhibitor or a combination thereof. In certain embodiments, the one or more chemotherapeutic agents include a platinum-based agent (e.g., cisplatin, carboplatin, or oxaliplatin), a topoisomerase II inhibitor (e.g., etoposide) or a combination thereof. In certain embodiments, the one or more chemotherapeutic agents comprise cisplatin or carboplatin and etoposide. In certain embodiments, the one or more chemotherapeutic agents are etoposide. In various embodiments, the one or more chemotherapeutic agents are administered according to dose and/or regimen approved by a regulatory agency (e.g., the FDA). For example, in various embodiments, carboplatin is administered at a dose sufficient to achieve AUC=5 mg/ml/min, and etoposide is administered at a dose of 100 mg/m².

In various embodiments, the anti-DLL3 antigen-binding molecule, the anti-PD-L1 antibody and the optional one or more chemotherapeutic agents are each administered by IV infusion. In certain embodiments, the anti-DLL3 antigen-binding molecule is administered after the administration of the anti-DP-L1 antibody and the one or more chemotherapeutic agents when given on the same day.

In certain embodiments, disclosed herein is a method of treating DLL3-expressing cancer (e.g., SCLC) in which at least 25% of the cancer cells express DLL3 or in which at least 25% of the cancer cells express DLL3 at an intensity of equal to or greater than 2+, comprising administering to a subject in need thereof an anti-DLL3 antigen-binding molecule and an alkylating agent, wherein the anti-DLL3 antigen-binding molecule is administered to the subject at a dose of from about 10 mg to about 200 mg once every three weeks. In certain embodiments, the anti-DLL3 antigen-binding molecule is administered at a dose of from about 10 mg to about 100 mg (e.g., 10 mg, 20 mg, 60 mg, or 100 mg) once every three weeks. In certain embodiments, the anti-DLL3 antigen-binding molecule is administered at a dose of from about 20 mg to about 200 mg (e.g., 20 mg, 60 mg, 100 mg, or 200 mg) once every three weeks. In certain embodiments, the anti-DLL3 antigen-binding molecule is administered on day 1 of a 21-day cycle. In various embodiments, the alkylating agent is lurbinectedin.

In certain embodiments, the method comprises administering to a subject in need thereof an anti-DLL3 antigen-binding molecule and an alkylating agent, wherein the anti-DLL3 antigen-binding molecule is administered to the subject in a 21-day cycle according to the following: a first dose of 0 mg or about 1 mg on day 1, a second dose of from about 10 mg to about 100 mg on day 8, a third dose of from about 10 mg to about 200 mg on day 15, and one or more subsequent doses of from about 10 mg to about 200 mg starting on day 22 and once every three weeks thereafter. In certain embodiments, the first dose is 1 mg, the second dose is from 10 mg to 100 mg (e.g., 10 mg, 20 mg, 60 mg, or 100 mg), the third dose is from 10 mg to 200 mg (e.g., 10 mg, 20 mg, 60 mg, 100 mg, or 200 mg), and the one or more subsequent doses are the same and are the same as the third dose (e.g., 10 mg, 20 mg, 60 mg, 100 mg, or 200 mg). In certain embodiments, the method comprises administering only the alkylating agent in cycle 1 and cycle 2 and administering the alkylating agent and the anti-DLL3 antigen-binding molecule in cycle 3 and thereafter. In various embodiments, the alkylating agent is lurbinectedin. In various embodiments, lurbinectedin can be administered according to a dose and/or regimen approved by a regulatory agency (e.g., the FDA), e.g., at a dose of about 3.2 mg/m², 2.6 mg/m², or 2 mg/m² once every three weeks.

In various embodiments, the anti-DLL3 antigen-binding molecule and the alkylating agent (e.g., lurbinectedin) are each administered by IV infusion. In certain embodiments, the anti-DLL3 antigen-binding molecule is administered after the administration of the alkylating agent when given on the same day.

Other cancer treatments that may be administered in combination with the antigen-binding-molecule described herein (optionally in combination with one or more chemotherapeutic agents and/or an anti-PD-L1 antibody) include, but are not limited to, surgery, radiation therapy, targeted therapy, immunotherapy, hormone therapy, and stem cell transplantation. Exemplary targeted cancer therapies include, but are not limited to, protein kinase inhibitors (e.g., BCR-ABL and c-KIT tyrosine kinase inhibitors, EGFR tyrosine kinase inhibitors, ALK tyrosine kinase inhibitors, V600E mutated-BRAF oncogene inhibitors, MEK inhibitors, Bruton kinase inhibitors, Janus kinase inhibitors, and CDK inhibitors).

### Additional Therapeutics

In some embodiments, the methods disclosed herein further comprise the use of one or more additional therapeutic agents to prevent, reduce or mitigate the risk of any adverse effects associated with the administration of the bispecific antigen-binding molecule (e.g., anti-DLL3 antigen-binding protein) alone, or in combination with the anti-PD-L1 antibody and/or chemotherapeutic agents. A major adverse effect that may be associated with the use of the anti-DLL3 antigen-binding protein is cytokine release syndrome (CRS). Additional therapeutic agents useful for preventing, reducing or mitigating the risk of CRS include, but are not limited to, corticosteroids (e.g., dexamethasone), fluid (e.g., saline), and anti-IL6 antibody (e.g., tocilizumab or siltuximab). Dexamethasone may be administered by IV administration prior to all cycle 1 doses of the anti-DLL3 antigen-binding protein (e.g., AMG 757) including all step doses, saline (e.g., 1 liter) may be administered IV following all anti-DLL3 antigen-binding protein (e.g., AMG 757) doses in cycle 1, and anti-IL6 antibody (e.g., tocilizumab or siltuximab) may be administered as needed (e.g., subject not responsive to IV fluid). Additional corticosteroid prophylaxis with oral dexamethasone may be implemented as needed. An exemplary dose of dexamethasone includes 8 mg/administration (maximum of 24 mg/day). An exemplary dose of tocilizumab includes 8 mg/kg (not to exceed 800 mg). Symptoms of CRS include fever, nausea, fatigue, headache, myalgias, and malaise, and therapeutic agents useful for treating such these symptoms (e.g., paracetamol/acetaminophen for fever) may also be used. In certain embodiments, additional therapeutic agents that may also be used for reducing or mitigating adverse effects associated with anti-DLL3 antigen-binding protein treatment include granulocyte colony-stimulating factor (e.g., filgrastim or pegfilgrastim).

Thus, in certain embodiments, the methods disclosed herein further comprise administering one or more additional therapeutic agents selected from a corticosteroid (e.g., prednisone, hydrocortisone, and dexamethasone), a fluid (saline), and anti-IL6 antibody (e.g., tocilizumab or siltuximab). In certain embodiments, the methods further comprise administering one or more additional therapeutic agents selected from a corticosteroid (e.g., dexamethasone), a fluid (saline) and tocilizumab or siltuximab. In certain embodiments, the one or more of the corticosteroid, fluid and tocilizumab are administered in cycle 1 wherein the anti-DLL3 antigen-binding protein (e.g., AMG 757) is administered.

### Therapeutic Response

The efficacy of the disclosed methods for treating cancer (such as SCLC) may be assessed by a variety of clinical outcomes, endpoints, and/or measures. In some embodiments, outcomes for a subject treated in accordance with the disclosed methods may be compared to a second SCLC subject having a lower DLL3 expression level and treated with the same bispecific antigen-binding molecule. In this regard, clinical outcomes that may be assessed include, but are not limited to, Progression Free Survival (PFS), Overall Survival (OS), Objective Response (ORR), Disease Control Rate (DCR), Duration of Response (DOR). The term “progression free survival (PFS),” as used herein, refers to the time from randomization until first evidence of disease progression or death. The term “overall survival (OS),” as used herein, refers to the time from randomization to death. The term “objective response rate (ORR),” as used herein, is a measure of how a specific treatment impacts tumor burden in a patient with a history of solid tumors and refers to the proportion of patients that respond either partially or fully to therapy. The term “duration of response (DoR),” as used herein, refers to the time from randomization to disease progression or death in patients who achieve complete or partial response. The term “disease control rate (DCR),” as used herein, describes the percentage of patients with advanced cancer whose therapeutic intervention has led to a complete response, partial response, or stable disease. In some aspects, the disclosed methods desirably increase one or more of Progression Free Survival (PFS), Overall Survival (OS), Objective Response Rate (ORR), and/or Disease Control Rate (DCR) and Duration of Response (DOR) as compared to a second SCLC subject having a lower DLL3 expression level and treated with the same antigen-binding molecule. Clinical endpoints with respect to cancer therapies are further described in, e.g., Delgado A. and Guddati, A. K., et al., Am J Cancer Res 2021; 11 (4): 1121-1131.

As discussed above, administration of the antigen-binding molecules described herein to DLL3-expressing cancer (e.g., SCLC) wherein the cancer cells expressing certain levels of DLL3 is advantageously associated with improved therapeutic outcomes, particularly as compared to standard of care (SOC). It will be appreciated that overall response rate (ORR) and median progression-free survival (PFS) for patients with SCLC receiving SOC second-line therapy is roughly 51% and 4.6 months, respectively. (Simos et al., Clin Lung Cancer, 15:110-118 (2014)). For patients with relapsed SCLC receiving SOC third-line chemotherapy, the ORR is approximately 18%, and the median PFS is approximately 2.0 months (Gong, J. and R. Salgia, J. Oncol. Practice, 14 (6): 359-366 (2018). Thus, in some embodiments, the methods described herein desirably increase ORR, PFS, and/or OS as compared to standard of care. For example, when the anti-DLL3 antigen-binding agent is administered as a first-line treatment for SCLC, the disclosed method desirably results in an ORR of at least about 65% (e.g., about 65% to about 70%), a median PFS greater than about 5 months (e.g., 7 months or more), and a median OS of at least about 13 months, such as about 13-15 months or about 16-18 months. When the anti-DLL3 antigen-binding agent is administered as a second-line treatment for SCLC, the disclosed method desirably results in an ORR of at least about 35%, a median PFS greater than about 7 months (e.g., 8 months or more), and a median OS of at least about 11 months (e.g., 11.5 months or more). When the anti-DLL3 antigen-binding agent is administered as a third-line treatment for SCLC, the method desirably results in an ORR of at least about 20% (e.g., about 24% or more), a median PFS greater than about 6 months, and a median OS of at least about 7.5 months (e.g., 8 months or more).

Ideally, the treatment provided by the methods of the present disclosure slow the progression of the cancer. For example, the methods can treat cancer by virtue of enhancing the T cell activity or an immune response against the cancer, reducing tumor or cancer growth, reducing metastasis of tumor cells, increasing cell death of tumor or cancer cells, and the like. In exemplary aspects, the methods may delay recurrence of the cancer by at least about 30 days, two months, 4 months, 6 months, 1 year, 2 years, 4 years, or more. In exemplary aspects, treatment may encompass increasing the survival of the subject. In various aspects, the treatment provided by the methods of the present disclosure includes a therapeutic response as per Response Evaluation Criteria in Solid Tumors (RECIST) or other like criteria. RECIST is a set of criteria to evaluate the progression, stabilization or responsiveness of tumors and/or cancer cells jointly created by the National Cancer Institute of the United States, the National Cancer Institute of Canada Clinical Trials Group and the European Organisation for Research and Treatment of Cancer. RECIST defines tumor size as the sum of longest diameters (SLD) of target lesions and categorizes patients into complete response (CR), partial response (PR), stable disease (SD), and progressive disease (PD). The CR and PR represent no new tumor lesions and a 100% shrinkage and greater than 30% shrinkage, respectively; PD represents a 20% increase in tumor size from the minimum size observed up to that point or new lesions appearing.

### Articles of Manufacture

Disclosed herein are articles of manufacture comprising: (a) a container comprising a bispecific antigen-binding molecule (e.g., an anti-DLL3 antigen-binding molecule such as AMG 757); and (b) a package insert with instructions for treating a DLL3-expressing cancer (or treating SCLC) in a subject by administering the anti-DLL3 antigen-binding molecule (e.g., AMG 757), wherein the instructions specify that at least 25% (e.g., 30%, 50%, 60%, 70% or 75%) of the cancer cells express DLL3 or at least 25% (e.g., 30%, 50%, 60%, 70% or 75%) of the cancer cells express DLL3 at an intensity of at least 2+, and that the anti-DLL3 antigen-binding molecule is administered at a dose of from about 10 mg to about 100 mg by intravenous (IV) infusion administered once every 2 weeks. In some embodiments, the instructions specify that at least 25% (e.g., 30%, 50%, 60%, 70% or 75%) of the cancer cells express DLL3 or at least 25% (e.g., 30%, 50%, 60%, 70% or 75%) of the cancer cells express DLL3 at an intensity of at least 2+, and that the anti-DLL3 antigen-binding molecule is administered at a dose of from about 10 mg to about 100 mg (e.g., 10 mg, 30 mg, or 100 mg) to the subject twice every three weeks, such as on day 1 and day 8 of a 21-day cycle. In certain embodiments, the instructions specify that at least 25% (e.g., 30%, 50%, 60%, 70% or 75%) of the cancer cells express DLL3 or at least 25% (e.g., 30%, 50%, 60%, 70% or 75%) of the cancer cells express DLL3 at an intensity of at least 2+, and that the anti-DLL3 antigen-binding molecule is administered at a dose of from about 20 mg to about 200 mg (e.g., 20 mg, 60 mg, 100 mg, 160 mg, or 200 mg) to the subject once every three weeks, such as on day 1 of a 21-day cycle. In certain embodiments, the instructions further specify that the anti-DLL3 antigen-binding molecule is administered by extended intravenous infusion over 2 to 7 days (e.g., over 3 days) in the first cycle in which the anti-DLL3 antigen-binding molecule is administered to the subject. In certain embodiments, the instructions further specify that the anti-DLL3 antigen-binding molecule is administered in cycle 1 (21 day cycle) according to a one step dosing regimen: 1 mg on day 1, a second dose on day 8 and a third dose on day 15, wherein the second and third doses are the same and are each from about 10 mg to about 100 mg.

In certain embodiments, articles of manufacture comprises: (a) a container comprising a bispecific antigen-binding molecule (e.g., an anti-DLL3 antigen-binding molecule such as AMG 757); and (b) a package insert with instructions for treating DLL3-expressing cancer (or treating SCLC) in a subject by administering the anti-DLL3 antigen-binding molecule (e.g., AMG 757) in combination with an anti-PD-L1 antibody (e.g., atezolizumab or durvalumab), wherein the instructions specify that at least 25% (e.g., 30%, 50%, 60%, 70% or 75%) of the cancer cells express DLL3 or at least 25% (e.g., 30%, 50%, 60%, 70% or 75%) of the cancer cells express DLL3 at an intensity of at least 2+, and that the anti-DLL3 antigen-binding molecule is administered at a dose of from about 20 mg to about 200 mg (e.g., 20 mg, 60 mg, 100 mg, 160 mg, or 200 mg) to the subject once every three weeks, such as on day 1 of a 21-day cycle. In certain embodiments, the instructions specify that at least 25% (e.g., 30%, 50%, 60%, 70% or 75%) of the cancer cells express DLL3 or at least 25% (e.g., 30%, 50%, 60%, 70% or 75%) of the cancer cells express DLL3 at an intensity of at least 2+, and that the anti-DLL3 antigen-binding molecule is administered at a dose of from about 10 mg to about 100 mg to the subject twice every three weeks, such as on day 1 and day 8 of a 21-day cycle. In certain embodiments, the instructions specify that at least 25% (e.g., 30%, 50%, 60%, 70% or 75%) of the cancer cells express DLL3 or at least 25% (e.g., 30%, 50%, 60%, 70% or 75%) of the cancer cells express DLL3 at an intensity of at least 2+, and that the anti-DLL3 antigen-binding molecule is administered at a dose of from about 10 mg to about 100 mg to the subject once every two weeks, such as on day 1 and day 15 of a 28-day cycle. In certain embodiments, the instructions further specify that one or more chemotherapeutic agents (e.g., carboplatin or cisplatin and/or etoposide) are administered to the subject in combination with the anti-DLL3 and anti-PD-L1 agents.

In certain embodiments, articles of manufacture comprises: (a) a container comprising a bispecific antigen-binding molecule (e.g., an anti-DLL3 antigen-binding molecule such as AMG 757); and (b) a package insert with instructions for treating a DLL3-expressing cancer (or treating SCLC) in a subject by administering the anti-DLL3 antigen-binding molecule (e.g., AMG 757) in combination with an alkylating agent (e.g., Lurbinectedin), wherein the instructions specify that at least 25% (e.g., 30%, 50%, 60%, 70% or 75%) of the cancer cells express DLL3 or at least 25% (e.g., 30%, 50%, 60%, 70% or 75%) of the cancer cells express DLL3 at an intensity of at least 2+, and that the anti-DLL3 antigen-binding molecule is administered at a dose of from about 20 mg to about 200 mg (e.g., 20 mg, 60 mg, 100 mg, 160 mg, or 200 mg) to the subject once every three weeks, such as on day 1 of a 21-day cycle.

In some embodiments, the package insert further comprises instructions for detecting and measuring human DLL3 protein expression in tumor cells from a subject. For example, the package insert desirably comprises instructions for measuring DLL3 expression by an immunohistochemical (IHC) assay, such as those described herein. In certain embodiments, the IHC assay has been approved by a regulatory agency, such as the U.S. Food and Drug Association (FDA) or an agency that has CE-IVD registration authority. In addition, the package insert may comprise instructions for scoring DLL3 expression intensity as measured by IHC in order to select subjects suitable for treatment, as well as appropriate doses of the anti-DLL3 antigen-binding molecule to administer to selected subjects. In various embodiments, the package insert may further instruct hospitalizing and monitoring the subject for up to about 48 hours (e.g., about 24 hours, 12 hours, 8 hours, 7 hours, 6 hours, 5 hours, 4 hours, 3 hours, 2 hours, or 1 hour) after the administration of the anti-DLL3 antigen-binding molecule (e.g., AMG 757) in cycle 1 and/or cycle 2. In various embodiments, the package insert may further instruct measuring or testing one or more cytokines such as IL-6, IL-8, IL-10, TNF-α, and IFN-γ of the subject (e.g., level of one or more cytokines in the subject's blood or serum) after the administration of the anti-DLL3 antigen-binding molecule (e.g., AMG 757) in cycle 1 and hospitalizing and monitoring the subject for up to about 48 hours (e.g., about 24 hours, 12 hours, 8 hours, 7 hours, 6 hours, 5 hours, 4 hours, 3 hours, 2 hours, or 1 hour) if any of the level of any of the cytokines is above a normal reference level. For example, the package insert may instruct measuring or testing IL-10 of the subject after the administration of the anti-DLL3 antigen-binding molecule (e.g., AMG 757) in cycle 1 and hospitalizing and monitoring the subject for up to about 48 hours (e.g., about 24 hours, 12 hours, 8 hours, 7 hours, 6 hours, 5 hours, 4 hours, 3 hours, 2 hours, or 1 hour) if the level of IL-10 is above a normal reference level. The cytokines can be measured or tested using methods known in the art.

In various embodiments, the container comprises the bispecific antigen-binding molecule (e.g., an anti-DLL3 antigen-binding molecule (e.g., AMG 757)) in an amount of about 1 mg, 5 mg, 10 mg, or 25 mg, for example, the anti-DLL3 antigen-binding molecule (e.g., AMG 757) is supplied as a sterile, single use, preservative free lyophilized drug product containing 1, 5, 10, or 25 mg of the anti-DLL3 antigen-binding molecule per container (e.g., vial). In various embodiments, the instructions specify that the lyophilized drug product is reconstituted with sterile water for injection. In various embodiments, the instructions specify that the subject is a human (e.g., a human having SCLC).

The following examples further illustrate the invention but, of course, should not be construed as in any way limiting its scope.

### Example 1

This example describes a phase 1 clinical study evaluating tarlatamab in patients with relapsed/refractory small cell lung cancer.

Small cell lung cancer (SCLC) is an aggressive lung cancer subtype with neuroendocrine differentiation diagnosed in more than 150,000 people worldwide each year. From 2001-2016, the 2-year survival rate was 11-17% for patients with SCLC in the US. The addition of immune checkpoint inhibitors atezolizumab or durvalumab to platinum and etoposide chemotherapy followed by maintenance therapy with checkpoint inhibitor alone as first-line treatment for SCLC has led to approximately 30% reduction in the risk of death and durable but modest survival gains for a subset of patients with extensive-stage SCLC (ES-SCLC). Available therapies are limited for the majority of SCLC patients who relapse. Topotecan, the most widely used second line agent globally, has limited efficacy and an unfavorable safety profile. In 2020 Lurbinectedin became the first drug approved by the FDA in over 20 years for second line therapy, and was conditionally approved based on an objective response rate (ORR) of 35%; however, a randomized study failed to demonstrate OS benefit. No agent is specifically approved for third-line treatment of relapsed SCLC.

The Notch signaling pathway is a regulator of neuroendocrine differentiation in SCLC. As discussed herein, the inhibitory Notch ligand delta-like ligand 3 (DLL3) is aberrantly expressed on the surface of up to 85% of SCLC cells and minimally expressed in normal tissues, making it a compelling therapeutic target. In vitro SCLC models have indicated a role for DLL3 in promoting tumor growth, migration, and invasion. The DLL3-targeted antibody-drug conjugate (ADC) rovalpituzumab tesirine showed clinical antitumor activity in patients with SCLC.

As discussed herein, tarlatamab, a half-life extended bispecific T cell engager (HLE BiTE®) molecule, binds both DLL3 on cancer cells and CD3 on T cells leading to T cell-mediated tumor lysis. Tarlatamab promotes tumor regression in preclinical models of SCLC. Tarlatamab is the first DLL3-targeted immune therapy to be evaluated clinically in SCLC.

The phase 1 trial evaluated, safety, pharmacokinetics, and preliminary efficacy of tarlatamab in patients with SCLC.

### Patients and Methods

### Study Design and Participants

DeLLphi-300 is a phase 1, multi-country, open-label, dose-escalation study evaluating tarlatamab monotherapy. Results reported here relate to a monotherapy regimen and include both dose escalation and expansion cohorts. Eligible patients were aged 18 years or older with histologically or cytologically confirmed SCLC who had progressed or recurred after at least 1 previous platinum-based regimen and if standard of care, a PD-L1 inhibitor in addition to chemotherapy. Included patients were required to have an Eastern Cooperative Oncology Group (ECOG) performance status of 2 or less and at least 2 measurable lesions defined per modified Response Evaluation Criteria in Solid Tumors (RECIST), version 1.1. Key exclusion criteria were untreated active brain metastases and severe or recurrent immune-mediated adverse events or infusion-related reactions while on prior immunotherapy.

The protocol and amendments were approved by the institutional review board or ethics committee at each participating site. The trial was conducted in accordance with the International Council for Harmonisation Good Clinical Practice guidelines and the principles of the Declaration of Helsinki. All patients provided written informed consent.

### Procedures

The planned tarlatamab dose levels were 0.003, 0.01, 0.03, 0.1, 0.3, 1, 3, 10, 30, and 100 mg by intravenous (IV) infusion administered every 2 weeks (Q2W). The first 4 dose levels were planned as single-patient cohorts as adverse events (AEs) were expected to be low. Tarlatamab administration continued until disease progression, unacceptable side effects, or consent withdrawal.

### Outcomes

The primary endpoint was safety including dose-limiting toxicities (DLT; defined as tarlatamab-related toxicity within 28 days after the first dose and meeting protocol criteria), AEs during the treatment period (treatment-emergent AEs [TEAEs]), and TEAEs possibly related to tarlatamab per investigator review (treatment-related adverse events [TRAEs]). Secondary endpoints included pharmacokinetics, antitumor activity by objective response per modified RECIST 1.1 (which applies aspects of immune-related response criteria including confirmation of disease progression to RECIST 1.1) by investigator assessment, duration of response (DOR), time to response (TTR), progression free survival (PFS), and overall survival (OS).

### Statistical Analysis

The analysis included patients enrolled in the escalation and expansion cohorts. The data cutoff was Jul. 19, 2022. A two-parameter Bayesian Logistic Regression Model (BLRM) model guided dose exploration. Safety data was reviewed on an ongoing basis. In dose level review meetings (DLRMs), the Sponsor, in consultation with site investigators, reviewed the BLRM recommended dose level and all available cumulative data by cohort prior to making dose escalation decisions. AEs and DLTs observed in all subjects were evaluated continually and fully integrated into all DLRMs. Based on the overall benefit-risk profile of 100 mg, it was decided to further evaluate this as the expansion dose. Descriptive statistics are provided for selected demographics, safety, pharmacokinetics (PK), pharmacodynamics, and biomarker data. An exploratory analysis was performed to evaluate the relationship between baseline expression of DLL3 and clinical benefit and is further detailed below. Kaplan-Meier methods were used to estimate the median and percentiles for time to event endpoints with confidence interval (CI) calculated using the Brookmeyer and Crowley method.

### Additional Description of Endpoints

Maximum tolerated dose (MTD) is the highest dose deemed safe as determined collaboratively by investigators and study team with consideration of the Bayesian Logistic Regression Model (BLRM).

Adverse events were graded using Common Terminology Criteria for Adverse Events (CTCAE), version 4.0. CRS events were graded using the Lee criteria.

Additionally, cytokine release syndrome (CRS), neutropenia, and neurologic events were monitored as events of interest in this study using an Amgen MedDRA Query narrow (AMQN) search approach. All events were coded using MedDRA version 24.1. Cytokine release syndrome by AMQN search includes Cytokine abnormal, Cytokine release syndrome, Cytokine storm, and Cytokine test. CRS events were graded using CRS Lee et al. (2014) criteria. Neutropenia was based on AMQN search and graded using CTCAE version 4.0. Neurologic Events were based on “Central neuropsychiatric events due to direct neurotoxicities” AMQN search and graded using CTCAE version 4.0.

Efficacy data disclosed herein were based on local investigators evaluations. Patients were defined as evaluable for efficacy if data cut-off date was at least 9 weeks after the first dose date to allow time for assessment.

Exploratory analyses of T cell and peripheral cytokines was performed on serially collected blood samples.

For immunogenicity assessments, blood samples from patients who received tarlatamab were collected on study day 1 (pre-dose) and multiple timepoints during the study for detection of anti-tarlatamab binding antibodies using a validated electro-chemiluminescent bridging immunoassay.

### Assessment of DLL3 Expression

Subjects enrolled in the study provided either a fresh biopsy or an archival formalin-fixed and paraffin-embedded (FFPE) tissue biopsy for retrospective DLL3 analysis by immunohistochemistry. Of the 107 subjects in the safety/efficacy population, 90 (84%) were evaluable for tumor DLL3 expression. DLL3 expression in tumor cells was detected by using an anti-DLL3 antibody (clone SP347 Ventana, Tucson, AZ). Briefly, FFPE SCLC tissue blocks were sectioned at 4-μm or 5-μm thickness on charged glass slides and stained on the BenchMark ULTRA (Ventana) using the U OptiView DAB IHC detection kit (Ventana) for visualization. Rabbit monoclonal was used as a negative marker control. The negative marker control for each specimen was evaluated for acceptable background and signal/noise staining. To evaluate DLL3 staining, combined membrane/cytoplasmic H-scores were collected at any magnification. Negative or weak staining was confirmed at a minimum of 20× magnification. Interpretation of DLL3 expression was performed by qualified pathologists. DLL3 positivity was defined as ≥1% stained tumor cells. Hematoxylin and Eosin (H&E) staining was performed on the Sakura TissueTek Prisma instrument. H&E-stained slides were used to evaluate for tumor content and assessment of tissue quality. A minimum of 100 viable tumor cells were required for biomarker evaluation.

Tumor tissues were scored for specific percentage tumor cell staining. The overall intensity of tumor cell staining on a scale of 0 to 3 in increments of 1 was also assessed in the DLL3 (SP347)-stained slides, as described in Huang et al., Arch Pathol Lab Med, 143 (11): 1373-137 (2019). Strong cytoplasmic and/or membranous staining of tumor cells was scored as “3” or “3+,”, moderate staining as “2” or “2+,” and weak staining as “1” or “1+.” Absence of staining was given a score of “0.”

### ROC Analysis

Receiver operating characteristic (ROC) analysis included 77 subjects enrolled in the 1-100 mg dose cohorts for whom pre-treatment DLL3 expression readouts were available. Total DLL3 expression (0%-100% tumor cell positivity for DLL3) at 10× magnification using the SP347 assay described above, along with exploratory scoring, was utilized retrospectively to examine the effect of patient selection on enrichment of clinical benefit (confirmed OR). True Positive Rate (TPR, y-axis) and False Positive Rate (FPR, x-axis) were computed across the full range of DLL3 expression. For each threshold considered, true positives were defined as DLL3-high responders, false positives were defined as DLL3-high non-responders, true negatives were defined as DLL3-low non-responders and false negatives were defined as DLL3-low responders.

### Immune Cell and Cytokine Analysis

Whole blood samples drawn into EDTA tubes were collected according to the schedule of assessment specified in the study protocol. A flow cytometry validated panel was used to stain whole blood samples using fluorescently labeled antibodies CD4 BV510 (clone SK3, BD Biosciences), CD8 BV605 (clone SK1, BD Biosciences), CD3 Alexa Fluor 700 (clone SK7, BioLegend), and CD279 (PD-1) BB515 (clone EH12.1, BD Biosciences). Data were acquired centrally at Q2 Solutions Laboratories Europe on a BD FACSCanto flow cytometer. To evaluate cytokine production, serum samples were collected and IFNγ levels were assessed using the Meso Scale Discovery (MSD) V-plex Pro-inflammatory Panel 1. The assay was performed according to the manufacturer's instructions. In brief, samples were diluted 1:2 with Diluent 2 (MSD). Diluted samples and standards were added in duplicate to a 96-well plate with capture antibodies independently precoated on 10 defined spots and incubated for 2 hours at ambient temperature. The plates were washed three times with wash buffer and a detection antibody mixture was added to each well and the plate was incubated for 2 hours at ambient temperature. The plate was washed three times with wash buffer and 2× Read Buffer T (MSD) was added to each well and read on an MSD plate reader. The concentrations were extrapolated from the standard curve within the established range of 2.61-542,720 μg/mL.

### Characterization of Cytokine Release Syndrome (CRS)

An analysis was performed in order to explore correlations between cytokine levels within 24 hours following the initial dose of tarlatamab and occurrence of CRS in Cycle 1. Cohorts receiving 1 mg as an initial dose of tarlatamab and from 1-100 mg in subsequent doses in Cycle 1 were included in this analysis.

Serum was drawn at time points up to 24 hours for cytokine analysis. The incidence, time to onset, severity, management, and recurrence of CRS were assessed. Serum peak level and elevation speed within 24 hours following the initial dose of tarlatamab were evaluated for a panel of soluble factors in patients with CRS versus no CRS in Cycle 1. Patients from 1 mg initial dosing cohorts were included.

A Kruskal Wallis (KW) rank-based test with false discovery rate correction was used to identify whether analyte values from the CRS and no CRS categories originate from different distributions. A Jonckheere-Terpstra (JT) trend test was utilized along with false discovery rate correction to identify increasing trend in analyte values from the no CRS to the CRS bucket. Univariate logistic regression was utilized to determine whether the growth rate and peak of each analyte was predictive of the occurrence of CRS in Cycle 1 post dosing.

### Results

### Patients

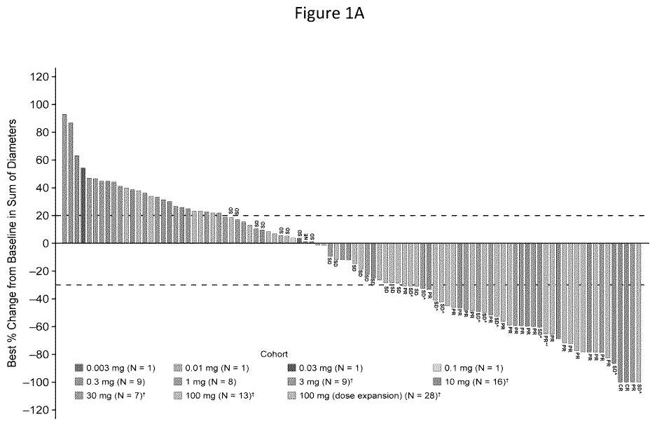

As of Jul. 19, 2022, 107 patients received tarlatamab in dose escalation (0.003-100 mg; n=73) and expansion (100 mg; n=34) cohorts (FIG. 1). Step-dosing was utilized starting with the 3 mg cohort (using 1 mg as the run-in dose followed by target dose on day 8, day 15, and Q2W thereafter) due to observed cytokine release syndrome (CRS) in prior cohorts. Cytokine release syndrome (CRS) is an expected risk of tarlatamab per its mechanism of action (MOA).

Baseline characteristics are summarized in Table 1. Median age was 63 years (range, 32-80). ECOG performance status was 0-1 in 99% of patients. More than 70% of patients had ≥2 lines of prior therapy, 25% were platinum refractory and 50% had prior PD-1/PD-L1 inhibitor.

The median follow-up was 8.7 months (range, 0.2-31.8). Treatment was discontinued in 92 patients (86%) most commonly for disease progression (n=77 [72%]). At data cutoff, 47 patients (43.9%) had ended study due to death. Median number of treatment cycles started was 3 (interquartile range [IQR]: 1, 8) and median number of tarlatamab doses received was 6 (IQR: 3, 16).

TABLE 1
Patient Demographics and Baseline Characteristics

| | All patients | |
|---|---|---|
| | (N = 107) | |
| Age, years | | |
| Median (IQR) | 63.0 | (58.0, 69.0) |
| Sex, n (%) | | |
| Male | 61 | (57%) |
| Female | 46 | (43%) |
| Race, n (%) | | |
| White | 86 | (80%) |
| Asian | 13 | (12%) |
| Black/African American | 3 | (3%) |
| Other | 5 | (5%) |
| Ethnicity, n (%) | | |
| Hispanic/Latino | 2 | (2%) |
| Not Hispanic/Latino | 105 | (98%) |
| Smoking history, n (%) | | |
| Never | 10 | (9%) |
| Current | 14 | (13%) |
| Former | 81 | (76%) |
| Missing | 2 | (2%) |
| ECOG performance status, n (%) | | |
| 0 | 40 | (37%) |
| 1 | 66 | (62%) |
| 2 | 1 | (1%) |
| Prior lines of therapy | | |
| Median (IQR) | 2.0 | (1.0, 3.0) |
| 1, n (%) | 30 | (28%) |
| 2, n (%) | 45 | (42%) |
| ≥3, n (%) | 32 | (30%) |
| Most recent line platinum-treated patients, n (%) | | |
| Platinum-sensitive | 54 | (51%) |
| Platinum-resistant | 22 | (21%) |
| Platinum-refractory | 26 | (25%) |
| Not Assessable/missing | 4 | (4%) |
| Prior radiotherapy, n (%) | | |
| Yes | 85 | (79%) |
| No | 22 | (21%) |
| Prior anti-PD-1 or anti-PD-L1, n (%) | | |
| Yes | 53 | (50%) |
| No | 54 | (51%) |
| Metastatic at baseline, n (%) | | |
| Yes | 100 | (94%) |
| Brain metastases | 27 | (25%) |
| Liver metastases | 54 | (51%) |
| No | 7 | (7%) |
| History of treated brain metastases | | |
| Yes | 39 | (36%) |
| No | 68 | (64%) |
| Extensive stage disease, n (%)* | 100 | (94%) |
| Sum of diameters of target lesions at baseline, mm | | |
| Median (IQR) | 75.0 | (43.0, 108.0) |

*Stage of disease unknown for 1 patient at baseline.
ECOG, Eastern Cooperative Oncology Group, IQR, interquartile range; PD-1, programmed cell death protein 1; PD-L1, programmed cell death ligand 1.

Retrospective DLL3 immunohistochemistry analysis was performed on fresh or archived biopsy as described above. DLL3 was expressed (≥1%) in 85 of 90 (94%) evaluable patients; median H-score was 186 (range, 0-300) and median tumor cell positivity was 95% (range, 0-100%).

### Safety and Tolerability

DLTs occurred in 6 patients including pneumonitis (n=1 [last prior dose, 0.3 mg]), increased alanine aminotransferase (n=1 [1 mg], CRS (n=1 [1 mg]), encephalopathy (n=1 [10 mg]), chills, pyrexia, and neutropenia (n=1 each [100 mg]). A maximum tolerated dose (MTD) was not reached; the highest dose (100 mg) was evaluated in the expansion cohort. Four patients (3.7%) discontinued tarlatamab due to AEs of encephalopathy (n=1), immune effector cell-associated neurotoxicity (ICANS) (n=1), and pneumonitis (n=2), all of which were treatment related. A single G5 pneumonitis event was recorded in a 70-year old male with a history of prior carboplatin/etoposide chemotherapy, chronic obstructive pulmonary disease, and radiation to the lung and pleural nodules. The event onset was cycle 1 day 18, 3 days after the second tarlatamab treatment (both doses 0.3 mg) and was confounded by clinically significant disease progression at the time of pneumonitis requiring urgent palliative radiation to the lung and to a soft tissue mass in the thoracic spine causing spinal cord compression. The cause of death was attributed by the investigator to disease progression and pneumonitis. An additional G3 and three additional G2 TEAEs of pneumonitis were observed (5/107 [4.7%] overall incidence of pneumonitis). Among patients with G2 pneumonitis, 1 patient ended treatment due to neurotoxicity (not pneumonitis), 1 patient had resolution of pneumonitis prior to discontinuation for PD, and 1 patient resumed treatment without dose change.

TEAEs of any cause/grade occurred in 107 patients (100%). The most common were CRS (56 patients [52.3%]), pyrexia (43 [40.2%]), constipation (33 [30.8%]) and fatigue (32 [29.9%]). Grade ≥3 AEs occurred in 61 patients (57.0%) with the most common being neutropenia (8.4%), decreased lymphocyte count (6.5%), and hypertension (5.6%). Serious adverse events (SAEs) occurred in 55 patients (51.4%). TEAEs led to dose reductions in 9 patients (8.4%) with 4 (3.7%) having CRS-related reductions. Dose interruption occurred in 20 patients (18.7%), most commonly for neutropenia and decreased neutrophil count. Any grade and grade ≥3 TRAEs occurred in 97 (90.7%) and 33 (30.8%) patients, respectively.

CRS, neutropenia, and neurologic events were monitored as events of interest based on preclinical, clinical, and mechanistic data with tarlatamab, other BiTE™ molecules, and other T cell-associated therapies. Amgen MedDRA Query narrow (AMQN) searches were performed to supplement standard system organ class single preferred term safety reporting (defined above and summarized in Table 2). Measures to ameliorate the potential for CRS included prophylactic corticosteroids (cycle 1 only) and IV hydration in some patients. Grade ≥2 treatment-emergent CRS was reported in 15 patients (14.0%) and grade 3 CRS in 1 patient (0.9%); no grade 4 or 5 CRS has been reported. For any grade CRS (n=56), median time to first onset was 2 days (range, 1-30 days) after first dose based on recorded date; more precise time-based reporting was implemented to better characterize CRS, with median time to onset of 17.5 hours in the subset of patients with hourly data available (n=47). CRS was transient (median duration, 3 days [IQR: 2-4 days]) and resolved in all cases. Eight patients (7.5%) received tocilizumab for CRS. CRS was largely confined to cycle 1. A total of 5 patients (4.7%) had CRS in cycle 2; 4 of these patients also had CRS in cycle 1, while one patient experienced CRS for the first time in cycle 2 or later. Treatment-emergent neurologic AEs of any grade occurred in 75 patients (70.1%) and were mostly grade 1; dysgeusia (29.0%), headache (19.6%), and dizziness (10.3%) were the most common. Grade ≥3 treatment-emergent neurologic events occurred in 12 patients (11.2%) including confusional state (4.7%), delirium (1.9%), and encephalopathy (1.9%). One subject had a grade 4 neurologic event (confusion), none had grade 5. All grade ≥3 neurologic AEs resolved, with 1 subject discontinuing tarlatamab due to G3 encephalopathy and 2 other subjects continuing treatment at reduced doses. G2 ICANS was the other neurologic cause leading to discontinuation in 1 subject. First onset of any grade neurological event was mostly within the first 30 days of treatment (median, 9 days [IQR, 2-29 days] with a median duration 5 days (IQR, 2-15 days). Grade ≥3 neutropenia occurred in 11 patients (10.3%). Any grade neutropenia first onset occurred at a median of 30 days (IQR, 21-31 days) after first tarlatamab administration, and median duration was 7 days (IQR, 4-13); overall, 10 patients (9.3%) received G-CSF. Febrile neutropenia occurred in 1 patient and was not considered treatment related.

TABLE 2
Treatment-emergent Adverse Events (Preferred term and AMQ for selected terms)

| All patients | | | | |
|---|---|---|---|---|
| (N = 107) | | | | |
| Any grade | Grade ≥2 | Grade ≥3 | Grade ≥4 | Grade 5 |

Adverse events of any cause that
occurred during treatment*

| | | | | | | | |
|---|---|---|---|---|---|---|---|
| Any | 107 (100%) | 94 (88%) | 61 (57%) | 13 | (12%) | 1 | (1%) |
| Serious | 55 (51%) | 44 (41%) | 30 (28%) | 7 | (7%) | 1 | (1%) |
| Resulting in discontinuation | 4 (4%) | 4 (4%) | 3 (3%) | 0 | (0.0) | 0 | (0.0) |

| Adverse events of any cause | | | | | | |
|---|---|---|---|---|---|---|
| that occurred during treatment | | | | | | |
| (any grade in >10% of patients | | | | | | |
| or grade ≥3 in >5%)* | | | | | | |
| Cytokine release syndrome | 56 (52%) | 15 (14%) | 1 (1%) | 0 | | 0 |
| Pyrexia | 43 (40%) | 11 (10%) | 2 (2%) | 0 | | 0 |
| Constipation | 33 (31%) | 8 (8%) | 0 | 0 | | 0 |
| Fatigue | 32 (30%) | 15 (14%) | 4 (4%) | 0 | | 0 |
| Nausea | 31 (29%) | 6 (6%) | 1 (1%) | 0 | | 0 |
| Dysgeusia | 31 (29%) | 6 (6%) | 0 | 0 | | 0 |
| Decreased appetite | 29 (27%) | 15 (14%) | 0 | 0 | | 0 |
| Anemia | 22 (21%) | 17 (16%) | 4 (4%) | 0 | | 0 |
| Headache | 21 (20%) | 1 (1%) | 0 | 0 | | 0 |
| Weight decreased | 19 (18%) | 8 (8%) | 1 (1%) | 0 | | 0 |
| Dyspnea | 18 (17%) | 8 (8%) | 4 (4%) | 0 | | 0 |
| Asthenia | 17 (16%) | 11 (10%) | 2 (2%) | 0 | | 0 |
| Vomiting | 16 (15%) | 3 (3%) | 1 (1%) | 0 | | 0 |
| Arthralgia | 15 (14%) | 2 (2%) | 0 | 0 | | 0 |
| Back pain | 14 (13%) | 7 (7%) | 0 | 0 | | 0 |
| Hypotension | 14 (13%) | 7 (7%) | 0 | 0 | | 0 |
| Cough | 14 (13%) | 2 (2%) | 0 | 0 | | 0 |
| Hypertension | 13 (12%) | 9 (8%) | 6 (6%) | 0 | | 0 |
| Neutropenia | 12 (11%) | 11 (10%) | 9 (8%) | 3 | (3%) | 0 |
| Hyponatremia | 12 (11%) | 7 (7%) | 5 (5%) | 0 | | 0 |
| White blood cell count decreased | 11 (10%) | 9 (8%) | 5 (5%) | 1 | (1%) | 0 |
| Diarrhea | 11 (10%) | 3 (3%) | 1 (1%) | 0 | | 0 |
| Dizziness | 11 (10%) | 2 (2%) | 0 | 0 | | 0 |
| Lymphocyte count decreased | 10 (9%) | 8 (8%) | 7 (7%) | 3 | (3%) | 0 |
| Adverse events of interest of | | | | | | |
| any cause^{†} | | | | | | |
| Neurologic events | 75 (70%) | 29 (27%) | 12 (11%) | 1 | (1%) | 0 |
| CRS | 56 (52%) | 15 (14%) | 1 (1%) | 0 | | 0 |
| Neutropenia | 17 (16%) | 16 (15%) | 11 (10%) | 4 | (4%) | 0 |

*Based on single preferred term incidence. Coded using MedDRA version: 25.0. Adverse events graded using CTCAE version 4.0 and CRS events graded using Lee et al. (2014) criteria.
^{†}CRS based on AMQ narrow search, which includes Cytokine abnormal, Cytokine release syndrome, Cytokine storm, Cytokine test. Neutropenia based on AMQ narrow search. Neurologic Events based on “Central neuropsychiatric events due to direct neurotoxicities” AMQ narrow search. Coded using MedDRA version: 25.0. CRS events graded using CRS Lee et al. (2014) criteria. Neutropenia and neurologic events graded using CTCAE version 4.0.
AMQ, Amgen MedDRA Query.

### Efficacy

Confirmed ORR was 23.4% (95% confidence interval [CI]: 15.7, 32.5) including 2 complete and 23 partial responses (Table 3).

TABLE 3
Tumor Response to Tarlatamab According
to Investigator Assessment

| | Interim efficacy analysis set* | |
|---|---|---|
| Response | (N = 107) | |
| Objective response rate, % (95% CI) | | |
| Confirmed | 23% | (15.7-32.5) |
| Confirmed and unconfirmed | 25% | (17.3-34.6) |
| Disease control rate, % (95% C1) | 51% | (41.5-61.2) |
| Best overall response, n (%) | | |
| Confirmed complete response | 2 | (2%) |
| Confirmed partial response | 23 | (22%) |
| Stable disease | 30 | (28%) |
| Progressive disease | 9 | (8%) |
| Could not be evaluated^{†} | 34 | (32%) |
| No assessment^{‡} | 9 | (8%) |
| Median time to response (IQR), months | 1.81 | (1.68-1.91) |
| Median duration of objective | 12.3 | (6.6-14.9) |
| response (95% CI), months | | |

*The Interim efficacy analysis set is a subset of the safety analysis set. The Interim efficacy analysis set include patients whose data cut-off date is at least 9 weeks after the first dose date.
^{†}This includes 32 patients who had PD in the post-baseline scan however without further confirmation scan (unconfirmed PD per modified RECIST 1.1).
^{‡}Reasons for no imaging assessment included consent withdrawn (n = 5), death (n = 2), clinical PD (n = 1), and starting new anticancer therapy (n = 1).

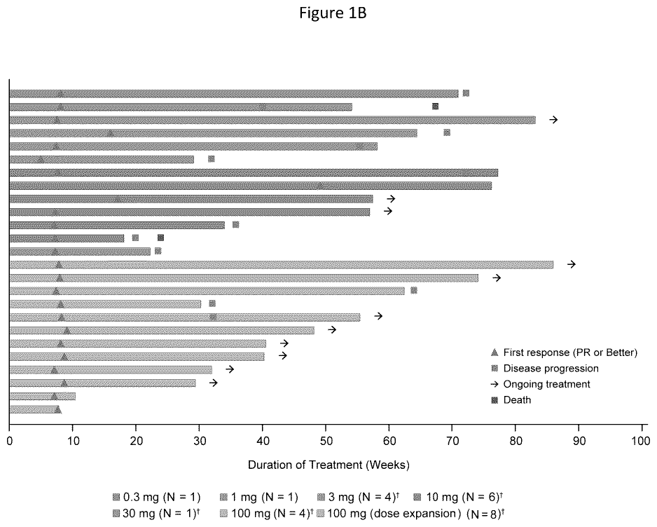

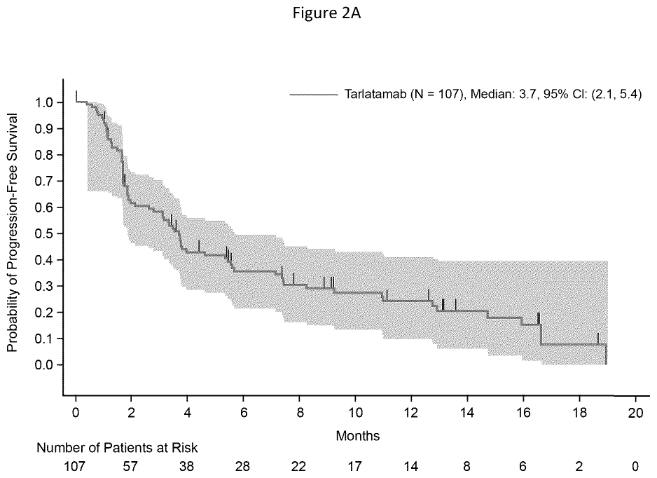

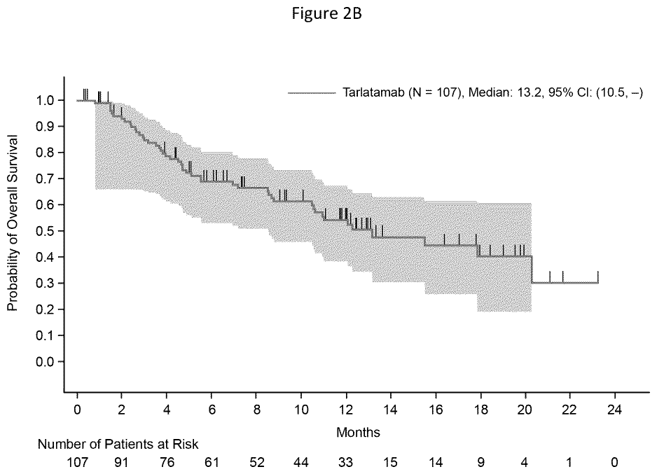

FIG. 1A illustrates best percentage change from baseline in sum of diameters for patients with evaluable postbaseline assessments (n=94). Disease control rate was 51.4% (95% CI: 41.5, 61.2). Responses were seen starting with the 0.3 mg dose and generally higher rates of response were observed at doses of 3 mg and above. At least 30% tumor shrinkage in target lesions at post-baseline assessment was observed in 39 patients (36.4%). Among confirmed responders, median TTR was 1.8 months (range, 1.2-7.4) and median DOR was 12.3 months (95% CI: 6.6, 14.9) (FIG. 1B). Longest duration of response was 14.9 months and 11 patients (44% of responders) had ongoing response at data cutoff. Median PFS was 3.7 months (95% CI: 2.1, 5.4) and median OS was 13.2 months (95% CI: 10.5, NE) (FIG. 2), respectively. A total of 28 patients (26.2%) went on to subsequent anticancer therapies after tarlatamab.

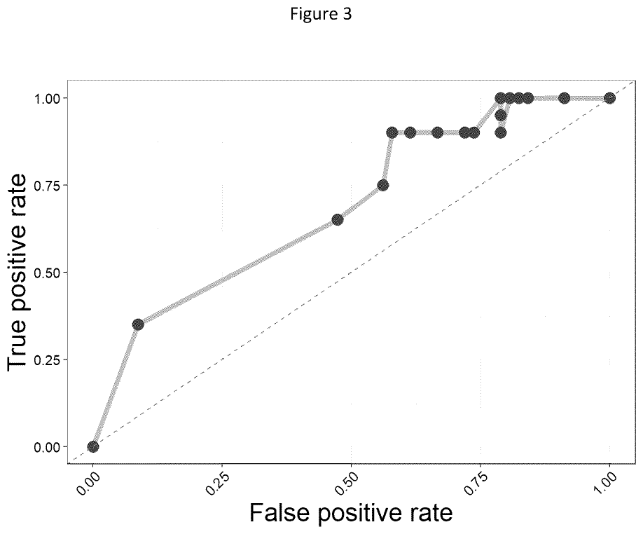

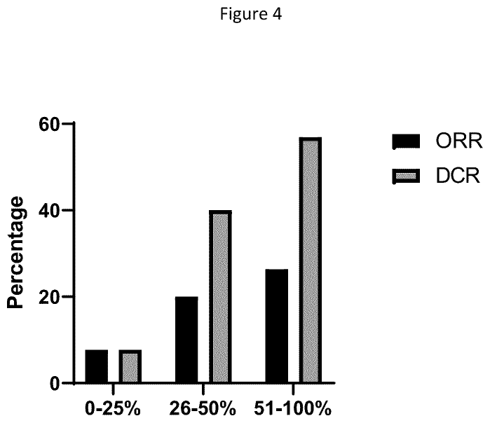

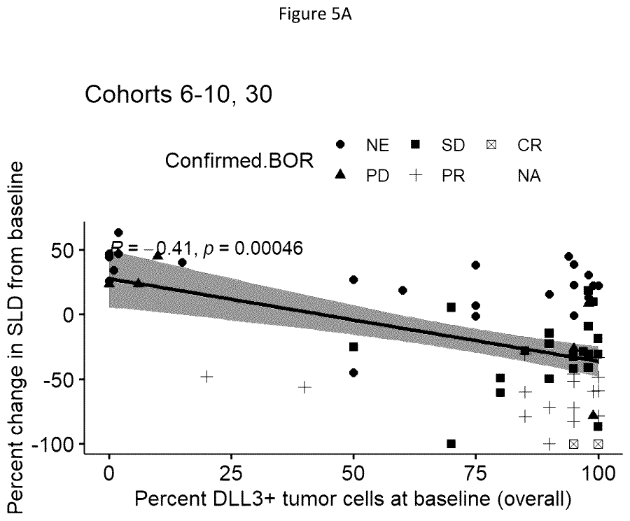

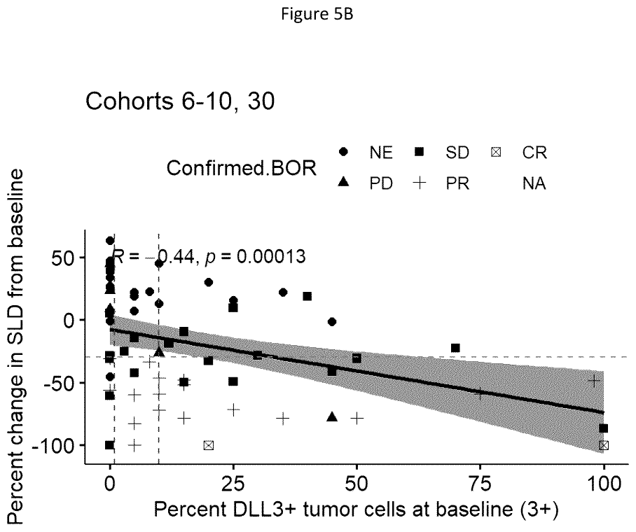

An analysis was conducted to investigate the relationship between DLL3 expression and clinical benefit with tarlatamab. FIG. 3 demonstrates the sensitivity and specificity of enriching for responders (using confirmed OR) when total DLL3 expression was retrospectively considered for patient selection. FIG. 4 demonstrates that DLL3 expression levels correlated with improved ORR and DCR. FIGS. 5A and 5B demonstrate a correlation between DLL3 expression and tumor reduction. Clinical benefit was observed across a range of thresholds.

### Clinical Pharmacokinetics

As of Apr. 15, 2022, preliminary pharmacokinetic data from dose escalation and expansion cohorts were available for 101 patients. Briefly, tarlatamab exhibited approximate dose proportional increase in serum exposures. Approximate steady state in serum tarlatamab exposures were achieved within 4 weeks of every-other-week target regimen initiation, with minimal accumulation. The mean (+SD) terminal elimination half-life estimated at steady-state across the evaluated target dose range was approximately 5.7 (+2.2) days, which is consistent with the intended half-life extension of the HLE platform relative to non-HLE BiTE™ molecules.

### Immunogenicity

Among the patients with available samples, 10 of 97 (10.3%) developed anti-tarlatamab antibodies after tarlatamab administration. Two of 99 (2.0%) patients had pre-existing antibodies at baseline. There was no apparent anti-drug antibody (ADA) impact on tarlatamab exposures or on the safety profile in these patients.

### Pharmacodynamics

The pharmacodynamic response after the first dose of tarlatamab infusion was characterized by initial T cell redistribution, T cell activation, and transient IFN-gamma elevation. For step-dose cohorts, pharmacodynamic responses were greatest after initial administration of 1 mg step dose and were not exceeded with target dose administration.

### Clinical CRS Summary

CRS was mostly grade 1 (39%), occurred in Cycle 1, and was reversible in all patients (see Table 4). CRS was clinically manageable.

TABLE 4

| | |
|---|---|
| | All Patients |
| Clinical CRS* Summary | (N = 106) |

| | | |
|---|---|---|
| CRS, n (%) | 56 | (53) |
| Grade 1 | 41 | (39) |
| Grade 2 | 14 | (13) |
| Grade 3 | 1 | (1) |
| Occurred in Cycle 1^{†} | 55 | (98) |
| Occurred in Cycle ≥2^{†} | 5 | (9) |
| Received oxygen for CRS^{†} | 3 | (5) |
| Intravenous fluid use for hypotension in CRS^{†} | 8 | (14) |
| Vasopressor use for hypotension in CRS^{†} | 2 | (4) |
| Received tocilizumab for CRS^{†} | 8 | (14) |
| Resolution^{†} | 56 | (100) |
| Median (range) time of CRS onset, h^{‡} | 17.5 | (3, 683) |
| Median (range) duration of CRS, d | 3 | (1, 22) |

To mitigate the risk of CRS, ≥1 of the following prophylactic measures were able to be used during cycle 1: additional corticosteroid prophylaxis with oral dexamethasone, administration of tocilizumab, etanercept, or acetaminophen.
*CRS includes cytokine abnormal, cytokine release syndrome, cytokine storm, cytokine test.
^{†}Percentage based on the total number of patients with any grade CRS.
^{‡}Time of onset in patients with both date and hour of onset data (n = 47).
Data cutoff: Jun. 15, 2022.
CRS, cytokine release syndrome.

### Cytokines and CRS Analysis

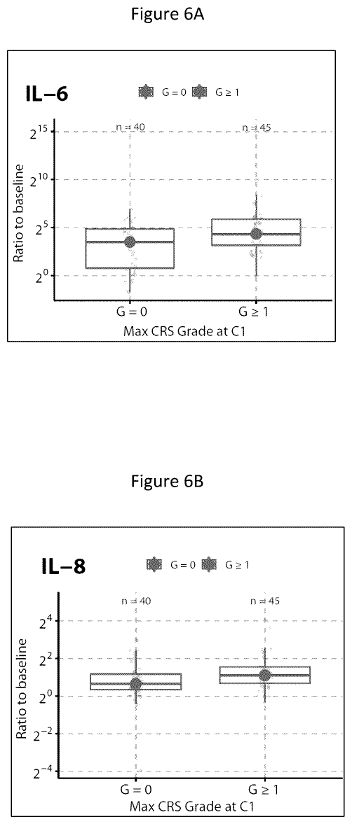

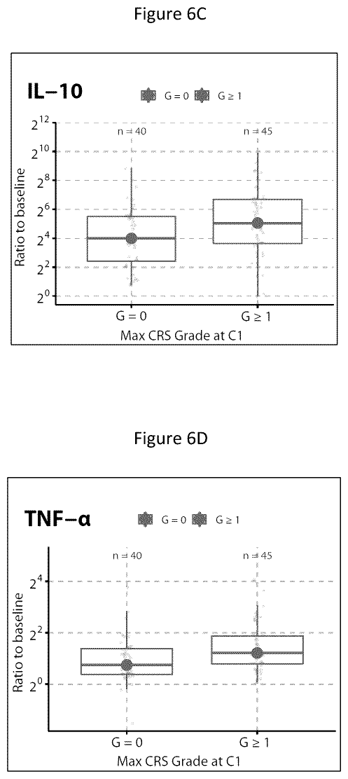

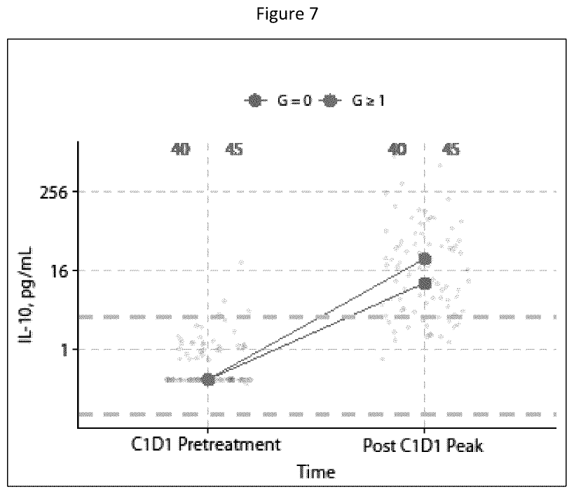

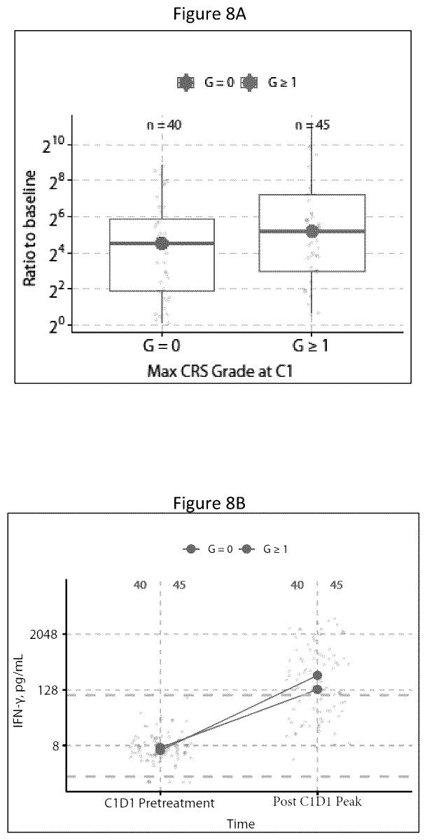

In biomarker evaluable patients, the ratio of peak level within 24 hours to baseline level for IL-6, IL-8, IL-10, and TNF-α trended higher in the patients with CRS in Cycle 1 versus patients without (FIGS. 6A-6D). IL-10 showed significant elevation above referenced normal range, and was higher in patients with CRS (FIG. 7). The effector cytokine IFN-γ was investigated due to its strong induction in preclinical experiments (see, e.g., Giffin M J, et al. Clin Cancer Res. 2021; 27:1526-1537). As expected from tarlatamab's mechanism of action, IFN-γ induction was above the physiologic range; induction was similar between those with Cycle 1 CRS versus those without Cycle 1 CRS (FIGS. 8A and 8B).

### Discussion

Tarlatamab demonstrated a manageable safety profile across a wide dose range through the expansion dose of 100 mg and was associated with encouraging response rates in a heavily pretreated population of SCLC patients. Confirmed responses were durable and OS appeared promising. Across all doses (N=107), tarlatamab was discontinued in only 4 patients (3.7%) and dose reductions were implemented for 9 patients due to AEs. An MTD was not reached; the highest dose (100 mg) was further evaluated in the dose expansion cohort.

CRS was expected based on the MOA of tarlatamab. While CRS was the most frequent TEAE observed in this study (56% of patients), it was generally low-grade, transient, and typically occurred in the first cycle. CRS was typically reversible and managed with steroids, IV fluids, and anti-pyretics, with tocilizumab used to treat CRS in 8 out of 107 patients receiving tarlatamab (7.5%). Neutropenia was a risk associated with tarlatamab observed in this study and was unexpected based on pre-clinical data; the mechanism is not understood. The study protocol was updated accordingly for specific monitoring and management. Further evaluation of neutropenia will be relevant to trials of tarlatamab use in combination with other marrow suppressing therapies. Neurologic evaluation was conducted as part of frequent clinical evaluation to assess study patients for CRS and/or neurologic AEs due to the known association with immune-effector cell therapies. Most neurologic AEs were mild and self-limiting without the need for treatment discontinuation or dose reduction, though there were 12 patients (11.2%) with grade ≥3 neurologic AEs. There were 2 patients who discontinued tarlatamab due to neurologic AEs (encephalopathy, ICANS). Careful evaluation of neurologic AEs is ongoing to better characterize these events and identify risk factors or interventions that might specifically improve management.

There are few approved therapies for SCLC after first line. A phase 2 study of lurbinectedin in second line SCLC found an ORR of 35% and median DOR of 5.3 months. In a randomized study of topotecan vs combination chemotherapy in recurrent SCLC, topotecan ORR was 24% and median DOR was 3.3 months. The prior conditional approval by US FDA of nivolumab and pembrolizumab for third line or later SCLC was based on response rates of 12% and 19%, respectively, with durable responses seen at ≥12 months in >60% of responding patients. These approvals were subsequently withdrawn as survival benefit was not demonstrated. The ORR of 23% and median DOR of 12.3 months for tarlatamab compares well with other therapies, especially considering over 70% of patients had at least 2 prior lines of therapy. Half of the patients in this study (50%) had received prior PD-1/PD-L1 therapy, representative of current practice in first line SCLC. Despite the median PFS (3.7 months) seen with tarlatamab, the median OS (13.2 months) is relatively high and compares favorably with 9.3-month median OS reported previously with lurbinectedin or about 6-month OS with topotecan, though the value of comparisons is limited by differences in study design and patient populations. The promising OS benefit may reflect the long durability of response seen thus far in those who respond to tarlatamab, but further follow up is needed in larger randomized studies. An alternative explanation of the relatively long OS with a short PFS could be that OS benefit derived from post-tarlatamab treatment, though this is less likely a major factor because only 26.2% of patients received such treatment in this heavily pretreated cohort. Identifying clinical, demographic, and biological factors predictive of response and/or toxicity (e.g., prior therapies, DLL3 expression) is an ongoing effort. Increased DLL3 expression appears to trend with a higher magnitude of clinical benefit.

The results of this example demonstrate promising activity of tarlatamab in patients with a high unmet medical need, and have led to several ongoing investigations of tarlatamab as monotherapy in SCLC and other neuroendocrine cancers.

### Example 2

This example describes a phase 2 study evaluating the efficacy, safety, tolerability, and pharmacokinetics of AMG 757/tarlatamab in subjects with relapsed/refractory small cell lung cancer after two or more prior lines of treatment.

### Study Design

The DeLLphi-301 study (20200491) is a phase 2, open-label, global study of AMG 757/tarlatamab in subjects with recurrent SCLC who have progressed or recurred following 1 platinum-based regimen (with or without checkpoint inhibitor) and at least 1 other line of therapy (re-treatment with a platinum-based regimen was considered a second line of therapy).

This study was conducted in 3 parts. Part 1 was a dose comparison to randomize approximately 180 patients in a 1:1 ratio to receive either 10 mg or 100 mg of tarlatamab (as a 60-minute intravenous infusion). A prespecified interim analysis was used to select the dose for parts 2 and 3. Part 2 was a dose expansion to enroll approximately 100 total patients (part 1 and 2 combined) at the selected dose. Part 3 was a substudy conducted after part 2 enrollment was completed to evaluate the safety of tarlatamab with reduced inpatient monitoring (from 48 to 24 hours) during cycle 1.

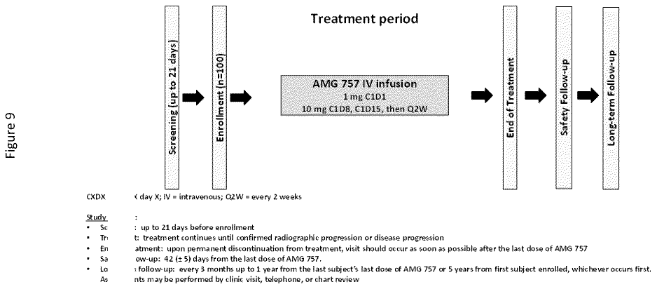

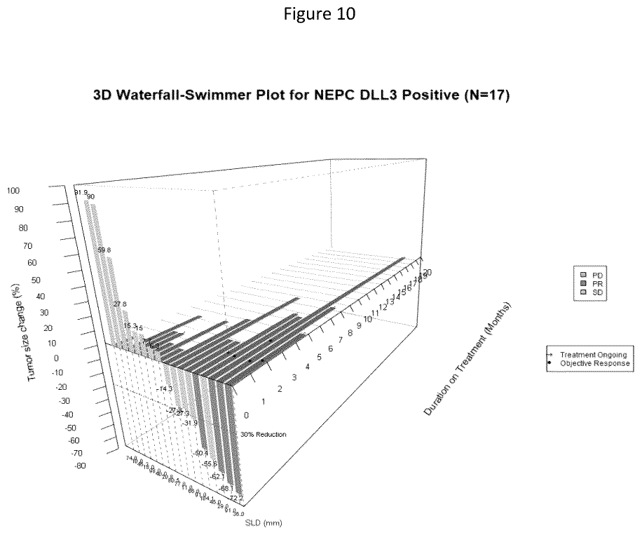

The primary efficacy endpoint was confirmed objective response (complete response or partial response) as per RECIST 1.1 by blinded independent central review (BICR). Secondary endpoints included DoR, disease control rate, duration of disease control, progression-free survival (PFS), overall survival (OS), incidence of treatment-emergent adverse events (TEAEs), serum concentrations of tarlatamab, and incidence of anti-tarlatamab antibody formation. Exploratory endpoints included cytokine levels, target expression in tumor tissue, immune-related biomarkers, and change from baseline in health-related quality of life. DLL3 immunohistochemistry was performed retrospectively on formalin-fixed, paraffin-embedded tissue at Roche Tissue Diagnostics (Tucson, USA) using the SP347 antibody. Objectives and endpoints of the study are shown in Table 5. The study schema is shown in FIG. 9.

TABLE 5

| | |
|---|---|
| Objectives | Endpoints |

Primary (Part 1 Only)

| | |
|---|---|
| Part 1 only: To evaluate safety and | objective response (OR) (complete |
| efficacy (per Response Evaluation | response [CR] and partial response |
| Criteriain Solid Tumors version 1.1 | [PR]) |
| [RECIST 1.1] by investigator) of 2 | incidence of treatment-emergent |
| dose levels of tarlatamab | adverseevents (TEAEs) |
| | serum concentrations of tarlatamab |

Primary (All Parts)

| | |
|---|---|
| Evaluate anti-tumor activity of | OR (CR and PR) |
| tarlatamab as determined by objective | |
| response rate(ORR) per RECIST 1.1 | |
| by blinded independent central review | |
| (BICR) | |

Secondary (All Parts)

| | |
|---|---|
| Evaluate anti-tumor activity of | duration of response (DOR) |
| tarlatamab as determined by other | disease control (DC) |
| measures per RECIST 1.1 by BICR | duration of DC |
| | progression-free survival (PFS) |
| Evaluate anti-tumor activity of | OR |
| tarlatamab as assessed by ORR and | DOR |
| other measures per RECIST 1.1 by | DC |
| investigator | duration of DC |
| | PFS |
| | overall survival (OS) |
| Evaluate the safety and tolerability of | incidence of TEAEs |
| tarlatamab | |
| Characterize the pharmacokinetics | serum concentrations of tarlatamab |
| (PK)of tarlatamab | |
| Evaluate the immunogenicity of | incidence of anti-tarlatamab |
| tarlatamab | antibody formation |

Exploratory (All Parts)

| | |
|---|---|
| Evaluate exploratory biomarkers | cytokine levels, target expression |
| including pharmacodynamic and | in archival tumor tissue or biopsy tumor |
| potential patient selection biomarkers | tissue, immune related biomarkers in |
| | blood and/or tumor tissue |
| Evaluate the treatment effect of | change from baseline over time in |
| tarlatamab on health-related quality of life | disease-related symptoms of: |
| (HRQoL) and disease symptoms and | dyspnea as measured by a 4 item |
| functional outcomes | dyspnea domain from QLQ-C30 and |
| | QLQ-LC13 |
| | cough as measured by QLQ-LC13 |
| | Chest Pain as measured by |
| | QLQ-LC13 |
| | change from baseline over time in: |
| | physical functioning as measured by |
| | QLQ-C30 |
| | global health status as measured by |
| | QLQ-C30 |
| | remaining subscales for QLQ-LC13 |
| | and QLQ-C30 |
| | visual analogue scale (VAS) scores |
| | as measured by |
| | EuroQol - 5-Dimension 5-Level |
| | (EQ-5D-5L) |
| | patient-reported adverse events |
| | based on selected questions from |
| | Patient Reported Outcomes Version |
| | of the Common Terminology Criteria |
| | for Adverse Events (PRO-CTCAE) |
| | and symptom burden as measured by |
| | the GP5 question from the Functional |
| | Assessment of Cancer |
| | Therapy - General form (FACT-G) |
| | time to deterioration (TTD) for the |
| | subscales of QLQ-LC13 and QLQ-C30 |
| Evaluate medical resource utilization | medical resource utilization, including |
| | inpatient hospitalizations and utilization |
| | related to cytokine release syndrome |
| | (CRS) |

### Treatments

For all 3 parts, tarlatamab was initiated with a step dose of 1 mg on cycle 1 day 1 (CID1), followed by either 10 mg or 100 mg on CID8, C1D15, and every 2 weeks thereafter (28-day cycles). Patients were treated until disease progression. Imaging assessments were scheduled for every 6 weeks for the first year and then every 12 weeks thereafter. Treatment beyond radiologic disease progression was permitted for patients with continued clinical benefits at the discretion of the investigator, provided protocol-specified criteria were met. Safety follow-up occurred 6 weeks after the last dose of tarlatamab, and long-term follow-up occurred every 3 months for 1 year after the last dose of tarlatamab or 5 years after the first patient enrolled, whichever occurred first.

### Statistical Considerations

A 15% objective response rate (ORR) was pre-specified in the protocol as the historical control benchmark based on published literature (Ready et al., J Thorac Oncol, 14:237-44 (2019); and Chung et al., J Thorac Oncol, 15:618-27 (2020)). Assuming an ORR of 30%, a sample size of 100 patients at the target dose from parts 1 and 2 was chosen to provide approximately 92% probability that the 97.5% confidence interval (CI) lower bound of ORR would exceed 15%. A 97.5% CI was chosen for the primary endpoint to adjust for multiplicity of dose selection at the pre-specified interim analysis. The safety analysis set consisted of all patients (parts 1-3) who received at least one dose of tarlatamab. The efficacy analysis set included all patients in parts 1 and 2 who received at least one dose of tarlatamab and had at least one measurable lesion at baseline as per BICR. Patients from part 3 were not included in the efficacy analysis set, as these data were immature.

Confidence intervals of proportions were calculated using the Clopper-Pearson method. Time-to-event endpoints were estimated using the Kaplan-Meier method.

### Summary of Subject Eligibility Criteria

Male and female subjects (≥18 years of age [or legal adult age within country]) with histologically or cytologically confirmed relapsed/refractory SCLC who progressed or recurred following 1 platinum-based regimen and at least 1 other prior line of therapy (Note: [1] re-treatment with a platinum-based regimen was considered a second line of therapy; [2] platinum-based regimen followed by checkpoint inhibitor/anti-programmed death ligand 1 [PD-L1] as maintenance therapy was considered 1 line of therapy; [3] in countries where standard of care first line systemic treatment includes platinum containing chemotherapy in combination with PD-L1 inhibitor, it was required that subjects failed PD-L1 inhibitor as part of their first line systemic treatment or were ineligible to receive PD-L1 inhibitor therapy).

Once consented to the study, subjects provided a medical history and underwent screening safety tests to confirm all eligibility requirements of the study had been met. Subjects must have had measurable lesions as defined per RECIST 1.1 within 21 days prior to the first dose of tarlatamab and must have had adequate organ function.

### Investigational Products

A summary of the dosing and administration of tarlatamab is shown in Table 6.

TABLE 6

| Study Treatment Name | Amgen Investigational Product:^{a} Tarlatamab |
|---|---|
| Dosage Formulation | Tarlatamab is supplied as a sterile, single use, preservative free lyophilized |
| | drug product containing 1, 5, 10, or 25 mg of tarlatamab per vial. |
| Unit Dose Strengths/ | 1 mg step dose on cycle 1 day 1 |
| Dosage Levels, and | 10 or 100 mg target dose starting cycle 1 day 8, cycle 1 day 15, and Q2W |
| Dosage Frequency | thereafter |
| Route of Administration | IV infusion |
| Accountability | The dose, start date/time, stop date/time, and lot number of tarlatamab are to |
| | be recorded on each subject's eCRF(s). |
| Dosing Instructions | Tarlatamab will be administered as an IV infusion for 60 minutes followed |
| | by a flush. Investigational products will be administered at the study center |
| | by a qualified staff member. A physician must be available at the time of |
| | administration of investigational products. |
| Hospitalization and | Cycles 1 and 2: Study sites must have immediate access to a |
| Monitoring Guidance | medical intensive care unit staffed by critical care providers. If |
| | required for logistical reasons (e.g., long travel times), subjects |
| | may be hospitalized the day before start of dosing (day −1) of |
| | any cycle, as well as during the treatment period for required PK |
| | samples. |
| | Cycle 1: Hospitalization for 48 hours post tarlatamab infusion |
| | for cycle 1 day 1 and cycle 1 day 8 doses. Hospitalization is not |
| | required for cycle 1 day 15 unless the subject experiences any |
| | grade CRS or neurological events following cycle 1 day 1 or |
| | cycle 1 day 8 (a minimum of 48 hours hospitalization is required |
| | post tarlatamab infusion on cycle 1 day 15 if any grade CRS or |
| | neurological events occur following cycle 1 day 1 or cycle 1 |
| | day 8). Subjects may be discharged after this period if there are |
| | no signs and symptoms of CRS or other acute toxicites. If |
| | subjects are not hospitalized during cycle 1 day 15, subjects will |
| | be observed for 8 hours post tarlatamab infusion. |
| | Cycle 2: Hospitalization is not required for cycle 2 unless a |
| | subject experiences grade 2 or higher CRS or neurological |
| | events in cycle 1 (minimum of 24 hours hospitalization required |
| | post tarlatamab infusion on cycle 2 day 1 if grade 2 or higher |
| | CRS or neurological events are noted in cycle 1). The subjects |
| | may be discharged after this period if there are no signs and |
| | symptoms of CRS or other acute toxicites. If subjects are not |
| | hospitalized during cycle 2, subjects will be observed for 8 hours |
| | post tarlatamab infusion. |
| | Cycles 3 and 4: Subjects should be monitored for 4 hours; |
| | hospitalization is not required. |
| | Cycles 5 and beyond: Subjects should be monitored for at least |
| | 2 hours; hospitalization is not required. |

CRS = cytokine release syndrome; eCRF = electronic case report form; IV = intravenous; PK = pharmacokinetics; Q2W = every 2 weeks.
^{a}Tarlatamab will be manufactured and packaged by Amgen and distributed using Amgen clinical study drug distribution procedures.

### Pre-Medication

Dexamethasone 8 mg IV (or equivalent dose of other corticosteroids) was administered within 1 hour prior to tarlatamab infusion on days 1 and 8 of cycle 1 only.

Prophylaxis with IV hydration (1 L normal saline) was administered immediately following all tarlatamab doses in cycle 1.

### Biomarker Assessment During the Study

Blood Samples: peripheral blood, serum and plasma samples were collected for evaluation of exploratory biomarkers that may include, but are not limited to cytokines, DNA, RNA analysis and enumeration of circulating tumor cells. These samples may be used to help with understanding further subjects' disease and their response to treatment.

Flow Cytometry Whole Blood Samples: whole blood samples were collected for immunophenotyping flow cytometry to identify changes in peripheral blood cell subsets and activation status.

Archival and/or Fresh Pre-Dose Tumor Tissues: fresh pre-dose tumor tissue or archival tumor tissues were evaluated for baseline DLL3 expression by IHC and for other exploratory biomarkers that may predict response.

Subjects must have provided archived tumor tissue samples taken after their last cancer therapy (formalin fixed, paraffin embedded [FFPE] sample collected within 2 years) or willing to undergo pretreatment tumor biopsy. Subjects who did not have archived tumor tissue available after their last cancer therapy or were unable to undergo a pretreatment tumor biopsy due to extenuating circumstances (e.g., cannot be performed safely or inaccessible as determined by the investigator) may have enrolled without a tumor biopsy upon agreement between the investigator and medical monitor.

The corresponding pathology report was submitted for any biopsy provided for the study. Tumor biopsies at time of first radiological assessment and after end of treatment were optional.

### Results

### Patients

Between December 2021 and May 2023, a total of 222 patients were enrolled at 56 sites in 17 countries. In part 1, 176 patients were randomized to receive 10 mg (n=88) or 100 mg (n=88) of tarlatamab. Based on results from the prespecified interim analysis, the 10 mg dose was selected for part 2 (dose expansion; n=12) and part 3 (reduced inpatient monitoring period; n=34). At data cutoff (Jun. 27, 2023), the median duration of treatment was 5.1 months (range, 0.0-15.2) in the 10 mg group and 3.7 months (range, 0.0-15.2) in the 100 mg group. Baseline demographics and clinical characteristics were comparable between the two dose groups although the proportion of patients with brain metastases at baseline was higher in the 100 mg group.

### Efficacy

A total of 185 patients from Parts 1 and 2 received tarlatamab, had at least 1 measurable lesion at baseline, and were included in the efficacy analyses; the median follow-up time was 10.6 months (95% CI, 9.7-11.3). The ORR by BICR was 40.4% (97.5% CI, 29.4-52.2) in the 10 mg group and 31.4% (97.5% CI, 20.6, 43.8) in the 100 mg group. ORR was consistent across pre-defined subgroups including the presence of brain or liver metastases, sensitivity to platinum-based chemotherapy in first-line treatment, and prior PD-(L) 1 inhibitor therapy. The disease control rate was 70.7% (95% CI, 60.7, 79.4) in the 10 mg group and 62.8% (95% CI, 51.7, 73.0) in the 100 mg group. Most responses (61/67, 91.0%) were seen at the first planned evaluation at 6±1 weeks after tarlatamab initiation. The median DoR was not reached in either group (10 mg: 95% CI, 5.9 months—not evaluable; 100 mg: 95% CI, 6.6 months—not evaluable). Among all 67 responders from the 10 mg and 100 mg groups, 39 (58.2%) have at least 6 months of response duration and 37 (55.2%) still have ongoing responses at data cutoff. Response by investigator were consistent with those per central review.

The median PFS (mPFS) was 4.9 months (95% CI, 3.0-6.7) in the 10 mg group and 3.9 months (95% CI, 2.6-4.4) in the 100 mg group. At 6 and 9 months, the Kaplan-Meier estimates of PFS were 40.8% (95% CI, 30.6-50.7) and 28.5% (95% CI, 19.2-38.6) for the 10 mg group and 33.2% (95% CI, 23.0-43.8) and 25.5% (95% CI, 16.1-35.9) for the 100 mg group, respectively. Median overall survival (mOS) was 14.3 months (95% CI, 10.8—not estimable) in the 10 mg group and not reached (95% CI, 12.4—not estimable) in the 100 mg group. The Kaplan-Meier estimates of overall survival at 6 and 9 months were 73.4% (95% CI, 63.2-81.2) and 68.0% (57.1-76.6) in the 10 mg group and 71.4% (95% CI, 60.1-80.0) and 65.5% (95% CI, 53.8-75.0) in the 100 mg group, respectively. At last follow-up, 57.6% (57/99) in the 10 mg group and 51.7% (45/87) in the 100 mg group were still alive, with OS data yet to mature.

Immunohistochemistry analysis showed that DLL3 expression was detected in 96.2% (150/156) of patient samples. Analyses of ORR and DoR according to DLL3 expression levels, assessed by BICR, are shown in Table 7.

TABLE 7

| | | | Objective Response | | | | |
|---|---|---|---|---|---|---|---|
| Part 1 and Part 2 | | | Rate | Duration of Response | | | |
| Overall | | | Events[a]/ | | | Median | 6-Month |
| (N = 156 evaluable | Incidence | | Subjects (%) | Events[c]/ | | (95% CI) | KM Estimate, % |
| samples) | n (%) | | (95% CI)[b] | Subjects (%) | | (Month)[d] | (95% CI)[e] |
| Potential DLL3 | | | | | | | |
| cutpoint 1 | | | | | | | |
| <75% at moderate | 84 | (53.8) | 22/84 (26.2) | 11/22 | (50.0) | 7.6 (4.4, | 67.9 (44.1, |
| (2+) and strong (3+) | | | (17.2, 36.9) | | | NE) | 83.2) |
| staining intensity | 72 | (46.2) | 34/72 (47.2) | 13/34 | (38.2) | NE (5.6, | 64.7 (45.0, |
| ≥75% at moderate | | | (35.3, 59.3) | | | NE) | 78.8) |
| (2+) and strong (3+) | | | | | | | |
| staining intensity | | | | | | | |
| Potential DLL3 | | | | | | | |
| cutpoint 2 | | | | | | | |
| <25% at moderate | 33 | (21.2) | 6/33 (18.2) | 5/6 | (83.3) | 5.7 (2.7, | 50.0 (11.1, |
| (2+) and strong (3+) | | | (7.0, 35.5) | | | NE) | 80.4) |
| staining intensity | | | | | | | |
| ≥25% at moderate | 123 | (78.8) | 50/123 (40.7) | 19/50 | (38.0) | NE (6.9, | 68.2 (52.7, |
| (2+) and strong (3+) | | | (31.9, 49.9) | | | NE) | 79.5) |
| staining intensity | | | | | | | |

### Safety

The most common treatment-emergent adverse events (TEAEs) were cytokine release syndrome (CRS) (55.0%), decreased appetite (34.5%), pyrexia (34.1%), constipation (26.4%), and anemia (25.9%). Grade 3 or higher TEAEs occurred in 59.4% of patients in the 10 mg group and 64.4% in the 100 mg group. Grade 3 or higher treatment-related adverse events (TRAEs) occurred in 25.6% of patients in the 10 mg group and 33.3% of patients in the 100 mg group. TRAEs led to dose interruption and/or reduction in 12.8% (10 mg) and 28.7% (100 mg) of patients and discontinuation in 3.0% (10 mg) and 3.4% (100 mg) of patients. There was one (0.8%) grade 5 TRAE due to respiratory failure that occurred in the 10 mg group.

CRS was reported in 51.1% (68/133) of patients in the 10 mg group and 60.9% (53/87) of patients in the 100 mg group, was mostly grade 1 (10 mg: 40/133 [30.1%], 100 mg: 28/87 [32.2%]) or grade 2 (10 mg: 27/133 [20.3%], 100 mg: 20/87 [23.0%]), and was largely confined to the first two doses (cycle 1 day 1 [CID1] and cycle 1 day 8 [C1D8]). Grade 3 CRS occurred in 0.8% (1/133) of patients in the 10 mg group and in 5.7% (5/87) of patients in the 100 mg group. Among patients that experienced CRS, the most common symptoms were fever (96.7%) hypotension (19.8%), and hypoxia (17.4%). The median onset of CRS following the last tarlatamab dose was 13.1 hours (Q1, Q3: 7.8, 27.4). The median duration of CRS was 4 days (Q1, Q3: 2, 6) and was generally manageable with supportive care that included acetaminophen, IV hydration, and/or corticosteroid use. Infrequently, additional interventions included tocilizumab (10 mg: 7/133 [5.3%]; 100 mg: 9/87 [10.3%]), supplemental oxygen (10 mg: 11/133 [8.3%]; 100 mg: 8/87 [9.2%])), and/or vasopressor support (10 mg: 1/133 [0.8%]; 100 mg: 1/87 [1.1%]). CRS led to dose interruption and/or dose reduction more frequently in the 100 mg group versus the 10 mg group (8/87 [9.2%] versus 4/133 [3.0%]). Nearly all cases (98.4%) of CRS resolved.

Immune effector cell-associated neurotoxicity syndrome (ICANS) was graded according to the American Society for Transplantation and Cellular Therapy (ASTCT) 2019 consensus (Lee et al., Journal of the American Society for Blood and Marrow Transplantation, 25:625-38 (2019). Analyses of these events included ICANS terms as well as potentially associated neurologic adverse events. ICANS including associated neurologic events occurred in 11 patients (8.3%) in the 10 mg group and 24 patients (27.6%) in the 100 mg group. Grade ≥3 events were not seen in the 10 mg group (0%; 0/133) and observed in 4 patients (4.6%) in the 100 mg group. ICANS including associated neurologic events occurred mostly during cycle 1 with a median time to onset of 5 days. The most common manifestations were immune effector cell-associated toxicity syndrome (10 mg: 6/11 patients, 100 mg: 9/24 patients), muscular weakness (10 mg: 4/11 patients, 100 mg: 6/24 patients), aphasia (10 mg: 1/11 patients, 100 mg: 2/24 patients), and cognitive disorder (100 mg: 3/24 patients). ICANS including associated neurologic events led to dose interruption and/or dose reduction in 1 (0.8%) patient in the 10 mg group and 5 (5.7%) patients in the 100 mg group and treatment discontinuation in 1 patient each in both groups. The median time to resolution was 6.5 days (95% CI: 4.0, 17.0).

Neutropenia was observed in 17.3% of patients in the 10 mg group and 16.1% of patients in the 100 mg group. Grade 3 febrile neutropenia was observed in 1 patient in each dose group. Neutropenia or febrile neutropenia did not lead to treatment discontinuation in any patient.

A similar safety profile was observed in patients who received 24-hour or 48-hour inpatient monitoring following tarlatamab infusion in cycle 1.

### Pharmacokinetics

Tarlatamab exhibited approximately dose-proportional increase in serum exposures within the evaluated dose range. Steady-state serum tarlatamab exposures were achieved by cycle 2 Day 15. Following the 10 mg and 100 mg Q2W regimen, tarlatamab mean (SD) Ctrough values at steady state (cycle 2 day 15 predose) were 0.51 (0.22) μg/mL and 6.8 (3.4) μg/mL, respectively. The results were consistent with those observed in the phase 1 study described in Example 1 and Paz-Ares et al., J Clin Oncol, 41 (16): 2893-2903 (2023), and support the Q2W dosing interval.

### Immunogenicity

Among 199 patients with at least one reportable post-baseline immunogenicity assessment, treatment-emergent anti-tarlatamab binding antibody developed in 4 patients (4/119; 3.4%) in the 10 mg group and 3 patients (3/80; 3.8%) in the 100 mg group. None of these patients developed anti-tarlatamab neutralizing antibodies. The presence of anti-tarlatamab antibodies did not appear to impact drug exposure, efficacy, or safety.

### Example 3

This example describes a phase 3 study of tarlatamab compared with standard of care (SOC) chemotherapy in relapsed small cell lung cancer (SCLC).

Described below is an open-label, randomized, multi-center, phase 3 study that will evaluate efficacy and safety of tarlatamab compared with SOC therapy for the treatment of subjects with small cell lung cancer (SCLC) who have progressed after 1 prior line of platinum-containing therapy.

### Study Design

The study includes of a pre-screening period, a 21-day screening period, a treatment period, a safety follow-up (SFU) visit, and a long-term follow-up (LTFU) period.

Provision of an evaluable archival tissue sample (formalin-fixed, paraffin-embedded [FFPE] blocks collected within 5 years or slides sectioned within 30 days prior to signing the relevant informed consent), or new core needle biopsy for central evaluation is required for enrollment and subjects must consent to tumor tissue collection and testing. Tumor material collection may occur at any time after initial diagnosis, including prior to the 21-day screening window, provided the pre-screening informed consent is completed. In order for a subject to be eligible for study participation, tumor tissue evaluability must be confirmed prior to randomization.

Subjects will be randomized with a 1:1 allocation ratio to receive tarlatamab or approved SOC therapy (lurbinectedin or topotecan in the United States (US), Canada, Australia, Singapore, Korea; amrubicin in Japan; topotecan in all countries except Japan).

Randomization will be stratified by:
Prior anti-PD-(L) 1 exposure (yes vs. no)·
Chemotherapy free interval (≥180 days; <180 to ≥90 days; <90 days)·
Presence of brain metastases (yes vs. no)

Approximately 350 subjects per arm and a total of approximately 700 subjects will be enrolled. Primary efficacy endpoints include OS, and secondary efficacy endpoints include PFS.

### Study Population

### Inclusion Criteria

Subjects are eligible to be included in the study only if all of the following criteria apply: subject has provided informed consent prior to initiation of any study specific activities/procedures; age ≥18 years (or legal adult age within country, whichever is older) at the time of signing the informed consent; histologically or cytologically confirmed relapsed/refractory SCLC; Subject has progressed or recurred following 1 platinum-based regimen (documented first disease progression must be during or following first line platinum-based systemic chemotherapy for ES or LS disease; patients who received treatment for LS disease who recur are eligible; patients who received adjuvant platinum-etoposide after resection of their SCLC who recur are eligible; in countries where SOC first line systemic treatment for ES disease includes platinum containing chemotherapy in combination with PD-L1 inhibitor, it is required that patients have failed PD-(L) 1 inhibitor as part of their first line systemic treatment or are ineligible to receive PD-(L) 1 inhibitor therapy); provision of evaluable tumor sample for central testing (tumor sample must be either archival (FFPE blocks collected within 5 years, or slides sectioned within 30 days prior to signing the relevant informed consent), or a fresh core needle biopsy); measurable disease as defined per RECIST 1.1 within the 21-day screening period (screening scans performed as standard of care and prior to informed consent, may be used to confirm subject eligibility if completed within the 21 day screening period, provided that informed consent for the use of these scans is obtained prior to any transfer of data); Eastern Cooperative Oncology Group (ECOG) performance status of 0 or 1; minimum life expectancy of 12 weeks.

Subjects must have adequate organ function, defined as follows: Hematological function (absolute neutrophil count ≥1.5×10⁹/L; platelet count ≥100×10⁹/L; hemoglobin >9 g/dL (90 g/L)); coagulation function (prothrombin time (PT)/international normalized ratio (INR) and partial thromboplastin time (PTT) or activated partial thromboplastin time (APTT)≤1.5× institutional upper limit of normal (ULN). Subjects on chronic anticoagulation therapy who do not meet the criteria above are eligible to enroll); renal function (estimated glomerular filtration rate (eGFR) based on Modification of Diet in Renal Disease (MDRD) calculation >30 mL/min/1.73 m²); hepatic function (aspartate aminotransferase (AST) and alanine aminotransferase (ALT)<3×ULN (or <5×ULN for subjects with liver involvement); total bilirubin <1.5×ULN (or <2×ULN for subjects with liver involvement); for subjects with the potential to receive lurbinectedin as SOC, total bilirubin <1.5×ULN and any AST); pulmonary function (no clinically significant pleural effusion. Pleural effusion managed with indwelling pleural catheter (e.g., PleurX) are allowed; baseline oxygen saturation >90% on room air); and cardiac function (cardiac ejection fraction ≥50%, no clinically significant pericardial effusion as determined by an echocardiogram (ECHO) or multigated acquisition (MUGA) scan, and no clinically significant electrocardiogram (ECG) findings).

### Exclusion Criteria

Subjects are excluded from the study if any of the following criteria apply.

Disease Related: untreated or symptomatic central nervous system (CNS) metastases or leptomeningeal disease (subjects with asymptomatic CNS metastases are eligible if clinically stable for at least 4 weeks and do not require intervention (including use of corticosteroids); subjects with treated brain metastases are eligible provided the following criteria are met: definitive therapy was completed at least 2 weeks prior to first dose of study treatment (stereotactic radiosurgery completed at least 7 days prior to first dose of study treatment), any CNS disease is clinically stable, subject is off steroids for CNS disease (unless steroids are indicated for a reason unrelated to CNS disease), and subject is off or on stable doses of anti-epileptic drugs at least 7 days prior to first dose of study treatment); prior history of immune checkpoint inhibitors resulting in: any severe or life-threatening immune-mediated adverse event, history of immune-mediated encephalitis or other immune-mediated CNS event (any grade), Grade ≥2 immune-mediated recurrent pneumonitis, infusion-related reactions leading to permanent discontinuation of immunotherapy agent (exception: subjects with a history of immune checkpoint inhibitor-induced endocrinopathy which is clinically stable on replacement therapy).

Other Medical Conditions: active autoimmune disease that has required systemic treatment (except replacement therapy) within the past 2 years or any other diseases requiring immunosuppressive therapy while on study; active infection requiring systemic treatment or any uncontrolled infection. Simple urinary tract infection (UTI) and uncomplicated bacterial pharyngitis are permitted if responding to active treatment. Subjects requiring oral antibiotics who have been afebrile >24 hours, have no leukocytosis, nor any clinical signs of infection are eligible; history of solid organ transplantation; history of other malignancy within the past 2 years (exceptions: low-risk malignancy treated with curative intent and with no known active disease present for ≥1 year before enrollment and felt to be at low risk for recurrence per investigator discretion; adequately treated non-melanoma skin cancer or lentigo maligna without evidence of disease; adequately treated cervical carcinoma in situ without evidence of disease; adequately treated breast ductal carcinoma in situ without evidence of disease; prostatic intraepithelial neoplasia without evidence of prostate cancer; adequately treated urothelial papillary noninvasive carcinoma or carcinoma in situ); myocardial infarction and/or symptomatic congestive heart failure (New York Heart Association >class II) within 12 months prior to first dose of study treatment; history of arterial thrombosis (e.g., stroke or transient ischemic attack) within 12 months prior to first dose of study treatment; history of hypophysitis or pituitary dysfunction (any grade); known human immunodeficiency virus (HIV) infection, hepatitis C infection (subjects with hepatitis C that achieve a sustained virologic response after antiviral therapy are allowed), or hepatitis B infection (subjects with hepatitis B surface antigen (HBsAg) or core antibody that achieve sustained virologic response with antiviral therapy are permitted with a requirement for regular monitoring for reactivation for the duration of treatment on the study); receiving systemic corticosteroid therapy or any other form of immunosuppressive therapy within 7 days prior to first dose of study treatment (prophylactic dexamethasone required by the protocol and any anti-emetic therapies are allowed, low-dose corticosteroids (prednisone ≤10 mg per day or equivalent is permitted during the trial)); evidence of severe acute respiratory syndrome coronavirus 2 (SARS COV-2) infection (subject is eligible if no acute symptoms of coronavirus disease 2019 (COVID 19) within 14 days prior to first dose of study treatment (counted from day of positive test for asymptomatic subjects)); evidence of interstitial lung disease or active, non-infectious pneumonitis.

Prior/Concomitant Therapy: prior therapy with tarlatamab or any of the SOC chemotherapy included as part of this trial; prior therapy with any selective inhibitor of the DLL3 pathway; subject received more than one prior systemic therapy regimen for SCLC (platinum/etoposide/anti-PD-(L) 1 therapy followed by anti-PD-(L) 1 maintenance therapy is considered one regimen); prior anti-cancer therapy within 21 days prior to first dose of study treatment (exceptions: subjects who received conventional chemotherapy are eligible if at least 14 days have elapsed and if all treatment-related toxicity has been resolved to grade≤1, or to levels dictated in the eligibility criteria, before first dose of study treatment, with the exception of alopecia or toxicities considered irreversible (defined as having been present and stable for >30 days) which are not otherwise described in the exclusion criteria; prior palliative radiotherapy must have been completed at least 7 days before the first dose of study treatment); receiving anti-cancer therapy such as chemotherapy, immunotherapy, or targeted therapy (patients who are receiving adjuvant hormonal therapy for resected breast cancer may be considered (refer also to exclusion related to history of other malignancies)); any herbal or prescription/non-prescription medications known to inhibit membrane transporters P-gp and/or breast cancer resistance protein (BCRP) (including but not limited to cyclosporine, clarithromycin, itraconazole, or ketoconazole) within 7 days prior to the first dose of study treatment; any herbal or prescription/non-prescription medications known to be moderate or strong inhibitors of cytochrome P450 3A (CYP3A) enzymes (including but not limited to clarithromycin, itraconazole, ketoconazole) within 7 days prior to the first dose of study treatment; any herbal or prescription/non-prescription medications known to be moderate or strong inducers of CYP3A enzymes (including but not limited to efavirenz, phenobarbital, phenytoin, rifampin, St John's Wort) within 28 days prior to first dose of study treatment; subjects who have reached the limit dose of prior treatment with cardiotoxic drugs such as other anthracyclines (the total dose of daunorubicin hydrochloride is 25 mg/kg body weight, the total dose of doxorubicin hydrochloride is 500 mg/m2 body surface area, the total dose of epirubicin hydrochloride is 900 mg/m2 body surface area, the total dose of pirarubicin hydrochloride is 950 mg/m2 body surface area, etc.); major surgical procedures within 28 day prior to first dose of study treatment; treatment with live virus including live attenuated vaccination within 4 weeks prior to first dose of study treatment (inactive vaccination (e.g., non-live or nonreplicating agent), including COVID-19 vaccination, within 3 days of the first dose of tarlatamab is not allowed (Note: vaccination precautions for SOC therapies are based on regional prescribing information)).

Prior/Concurrent Clinical Study Experience: currently receiving treatment in another investigational device or drug study, or less than 30 days since ending treatment on another investigational device or drug study(ies). Other investigational procedures while participating in this study are excluded.

Other Exclusions: female subjects of childbearing potential unwilling to use protocol specified method of contraception during treatment and for an additional 72 days after the last dose of tarlatamab (Note: contraception requirements for SOC therapies are based on regional prescribing information); female subjects who are breastfeeding or who plan to breastfeed while on study through 72 days after the last dose of tarlatamab (Note: breastfeeding restrictions for SOC therapies are based on regional prescribing information); female subjects planning to become pregnant or donate eggs while on study through 72 days after the last dose of tarlatamab (Note: contraception requirements for SOC therapies are based on regional prescribing information); female subjects of childbearing potential with a positive pregnancy test assessed at screening by a serum pregnancy test; male subjects with a female partner of childbearing potential who are unwilling to practice sexual abstinence (refrain from heterosexual intercourse) or use contraception during treatment and for an additional 132 days after the last dose of tarlatamab; male subjects with a pregnant partner who are unwilling to practice abstinence or use a condom during treatment and for an additional 132 days after the last dose of tarlatamab; male subjects unwilling to abstain from donating sperm during treatment and for an additional 132 days after the last dose of tarlatamab; subject has known sensitivity to any of the products or components to be administered, or with the potential to be administered, during dosing; subject likely to not be available to complete all protocol-required study visits or procedures, and/or to comply with all required study procedures (e.g., Clinical Outcome Assessments) to the best of the subject and investigator's knowledge; history or evidence of any other clinically significant disorder, condition or disease (with the exception of those outlined above) that, in the opinion of the investigator or medical monitor, if consulted, would pose a risk to subject safety or interfere with the study evaluation, procedures, or completion.

### Standard of Care Treatment Options

Lurbinectedin will be dosed at the approved regimen of 3.2 mg/m2 on day 1 of each 21 day cycle. Lurbinectedin will be administered as an IV infusion for 60 minutes. Lurbinectedin is supplied as a sterile, preservative-free, white to off-white lyophilized powder containing 4 mg in a single-dose clear glass vial. Lurbinectedin is intended for reconstitution with Sterile Water for Injection USP. The lyophilized formulation contains sucrose, lactic acid and sodium hydroxide.

Topotecan will be dosed at the approved regimen of 1.5 mg/m2 IV or 2.3 mg/m2/day oral on days 1 to 5 of each 21-day cycle. Topotecan Injection will be administered as an IV infusion over 30 minutes. Topotecan capsules should be swallowed whole. Topotecan injection is supplied as a sterile, non-pyrogenic, clear, light yellow to greenish solution in a single-use vial containing free base concentration of 4 mg/4 mL (1 mg/mL). Each mL contains topotecan hydrocholoride (equivalent to 1 mg of topotecan free base), mannitol, USP, and tartaric acid, NF. It may also contain hydrochloric acid and sodium hydroxide to adjust the pH. Topotecan capsules are supplied as 0.25 mg opaque white to yellowish-white and imprinted with HYCAMTIN and 0.25 mg or 1 mg opaque pink and imprinted with HYCAMTIN and 1 mg. Capsules contain topotecan hydrochloride (the content of which is expressed as topotecan free base), with excipients of gelatin, glyceryl monostearate, hydrogenated vegetable oil, and titanium dioxide. Imprints are made with edible black ink. The 1 mg capsules also contain red iron oxide.

Amrubicin will be dosed at the approved regimen of 40 mg/m2 and will be administered as an IV infusion on days 1 to 3 of each 21 day cycle. Amrubicin is supplied as a yellowish red powder or mass in vial containing 20 mg or 50 mg amrubicin hydrochloride. Excipients include lactose hydrate, L-cysteine hydrochloride, monohydrate, hydrochloric acid, and sodium hydroxide.

### Tarlatamab Pre- and Post-Infusion Medications

Dexamethasone 8 mg IV (or equivalent dose of other corticosteroids) will be administered within 1 hour prior to tarlatamab infusion on days 1 and 8 of cycle 1 only.

Prophylaxis with IV hydration (1 L normal saline over 2 to 4 hours) will be administered immediately following all tarlatamab doses in cycle 1 only.

### Example 4

This example describes a phase 1b clinical study evaluating the safety, tolerability, pharmacokinetics, and efficacy of tarlatamab in subjects with de novo or treatment emergent neuroendocrine prostate cancer (NEPC).

Prostate cancer is the most frequently diagnosed non-cutaneous cancer in men, with an estimated 191,930 new cases and 33,330 deaths in the United States (US) in 2020 (American Cancer Society, 2020). In the European Union, there were an estimated 365,000 new cases of prostate cancer in 2015, with 72,000 and 77,000 deaths estimated in 2012 and 2015, respectively (10% of total cancer deaths) (Crocetti E., Epidemiology of prostate cancer in Europe; publications.jrc.ec.europa.eu/repository/handle/JRC101382 (2015)). NEPC represents an aggressive variant of prostate cancer. While de novo cases appear to be rare, accounting for less than 2% of patients at the time of initial diagnosis (Parimi et al., Am J Clin Exp Urol., 2:273-285 (2014)), treatment-emergent NEPC, characterized by a histological transformation from adenocarcinoma to a high-grade neuroendocrine tumor, is becoming increasingly recognized and may develop in 15% to 20% of patients treated with standard therapies for prostate adenocarcinoma, including novel hormonal therapies (Aggarwal et al., J Clin Oncol., 36:2492-2503 (2018)). The exact mechanisms underlying this histological transformation are unclear, however, loss of androgen signaling dependence and the acquisition of alternative lineage programs have been associated with the development of NEPC. Overall, the prognosis for NEPC is poor (Wang et al., J Clin Oncol., 32:3383-3390 (2014)) and is often treated with platinum-based chemotherapy regimens (Aparicio et al., Clin Cancer Res., 19:3621-3630 (2013)). There is currently no standard therapeutic approach for NEPC.

### Study Design

An open label phase 1b study evaluating tarlatamab monotherapy for NEPC was conducted. Tarlatamab was administered as a short-term intravenous (IV) infusion every 2 weeks (Q2W) with step dosing in a 28-day cycle in subjects with de novo or treatment-emergent NEPC.

Due to its known mechanism of action, subjects were at an increased risk for first dose effects (e.g., cytokine release syndrome (CRS)) following the initial infusion of tarlatamab. To mitigate these risks, a step dosing approach was implemented. The study consisted of a single-step dose exploration involving a run-in dose of 1 mg tarlatamab on day 1, followed by a single step to a target dose of 100 mg tarlatamab on day 15 then Q2W. The 100 mg dose of tarlatamab was the highest dose deemed safe and tolerable in the ongoing phase 1 trial of tarlatamab in subjects with SCLC. Approximately 40 subjects were enrolled in this study.

### Summary of Subject Eligibility Criteria

Adult subjects (≥18 years of age) with metastatic de novo or treatment-emergent NEPC defined as one or more of the following were eligible to enroll: histological diagnosis of small cell NEPC, prostate carcinoma with neuroendocrine differentiation as defined by positive immunohistochemical staining for chromogranin and/or synaptophysin in the majority of the tumor sample, or ≥2 alterations in Tp53, RB1, and/or PTEN by immunohistochemistry (IHC) or genomic analyses of baseline tumor tissue or circulating tumor DNA (ctDNA). Subjects were required to have progressed on at least 1 line of prior treatment, including a platinum containing regimen for de novo NEPC (if at the time of NEPC diagnosis they had no prior diagnosis or treatment for prostate carcinoma) or an androgen signaling inhibitor (e.g., abiraterone, enzalutamide, and/or apalutamide) if treatment-emergent (had a previous diagnosis of prostate carcinoma prior to NEPC diagnosis). Subjects must have had measurable disease per Response Evaluation Criteria in Solid Tumors (RECIST) 1.1 criteria with Prostate Cancer Working Group 3 (PCWG3) guidelines, an Eastern Cooperative Oncology Group (ECOG) performance status of ≤2, and adequate organ function.

### Treatments and Procedures

Tarlatamab was administered as a short-term IV infusion (approximately 60 minutes followed by a flush) on day 1 and day 15 in a 28-day cycle (cycle 2 and beyond). To reduce the risk of cytokine release syndrome (CRS), premedication with dexamethasone 8 mg IV (or equivalent dose of other corticosteroids) was administered within 1 hour prior to all cycle 1 doses. Prophylaxis with IV hydration 1 L normal saline over 4 to 5 hours immediately following all dose(s) in cycle 1 was required.

Sites were required to have tocilizumab or siltuximab (if tocilizumab not available) on site for potential treatment of CRS. Tarlatamab was dosed until disease progression (treatment beyond disease progression could be allowed per PCWG3 guidelines).

Each subject followed the same treatment schedule and procedural requirements. After written informed consent was obtained, all screening tests and procedures were performed within 28 days of administration of tarlatamab (day 1), unless otherwise noted. Serial clinical safety and study evaluations were performed, including physical examination, vital signs, clinical laboratory tests, radiological assessment, tumor biopsy, pharmacokinetics (PK), and biomarker sample collections.

All subjects were hospitalized for intensive monitoring for 48 hours post tarlatamab infusion during all cycle 1 doses. Hospitalization was not required for cycle 2 unless a subject experienced grade 2 or higher CRS or neurological events in cycle 1 (minimum of 24 hours hospitalization required post tarlatamab infusion on cycle 2 day 1 if grade 2 or higher CRS or neurological events were noted in cycle 1). The subjects could be discharged after this period if there were no signs and symptoms of CRS or other acute toxicityf subjects were not hospitalized during cycle 2, subjects were observed for 8 hours post tarlatamab infusion.

Routine radiological imaging (computed tomography [CT]/magnetic resonance imaging [MRI]) and tumor burden assessments were performed. Assessment of disease response was determined based on RECIST 1.1 per PCWG3 guidelines. To further assess the risk of delayed adverse events, the subject returned for a safety follow-up (SFU) visit approximately 42 (+5) days after the last dose of tarlatamab.

Long-term follow-up was conducted every 3 months up to 3 years from the first dose of tarlatamab for all subjects who did not withdraw consent by clinic visit, telephone, or chart review to assess for survival and/or the commencement of subsequent cancer therapy.

### Statistical Considerations

All subjects who were enrolled and received at least 1 dose of tarlatamab were included in the analysis, unless otherwise specified.

The primary analysis occurred when target enrollment was complete and each subject either completed at least 6 months on study or withdrew from the study. The final analysis was performed after the last subject had an opportunity to complete the corresponding end of treatment visit/procedures.

Descriptive statistics were provided for selected demographics, safety, pharmacokinetic, efficacy data by dose, dose schedule, and time as appropriate. Descriptive statistics on continuous data included means, medians, standard deviations and ranges, while categorical data was summarized using frequency counts and percentages. The number and percentage of subjects reporting any treatment-emergent adverse events were tabulated. Clinical laboratory tests, physical examination findings, and vital sign data were listed. Summaries of laboratory, examination, and vital sign data over time and/or changes from baseline over time were provided.

Confidence intervals (CI) for proportions were estimated using an exact method proposed by Clopper-Pearson (Clopper and Pearson, Biometricka, 26:404-413 (1934)). Kaplan-Meier methods were used to estimate the median and percentiles for time to event endpoints with CI calculated using the Brookmeyer and Crowley (Brookmeyer and Crowley, Biometrics, 38:29-41 (1982)) method. Kaplan-Meier methods were used to estimate landmarks for time to event endpoints with the Greenwood formula (Kalbfleisch and Prentice, The Statistical Analysis of Failure Time Data, John Wiley & Sons, Inc., New York, xi+321 pp (1980)) used to estimate the standard error used in CI calculation.

### Biomarker Assessment During the Study

Blood samples were collected for assessment of circulating markers including, but not limited to, PSA and cytokines. Blood samples also were collected for enumeration and phenotyping of circulating tumor cells (CTCs). These samples were used to help with understanding further subjects' disease and their response to treatment.

Whole blood samples were collected for immuno-phenotyping flow cytometry to identify changes in T-cell subsets and activation status at certain time points. Plasma and/or serum, or tissue may have also been used for DNA, RNA, and protein expression analysis including somatic mutations in order to correlate levels of expression with response.

Formalin fixed paraffin embedded (FFPE) archival tumor tissues were evaluated for baseline DLL3 expression by IHC and for other exploratory biomarkers that may predict response.

To meet eligibility requirements, subjects must have provided fresh tumor tissue for biomarker discovery and future research unless archival tissue was taken after the last cancer therapy. For archival tumor samples within the first week after enrollment, an archived FFPE tumor tissue collected prior to the study was assessed.

Tumor biopsies were collected and pharmacodynamic (PD) changes analyzed to determine the effect of the investigational product on target(s) in the tumor as well as to potentially analyze molecular mechanisms associated with acquired resistance.

### Results

A total of 40 patients received tarlatamab and 38 were included in the efficacy analyses per RECIST 1.1 criteria. Tarlatamab was safe and tolerable in NEPC subjects with an adverse event profile that was similar to the AE profile in SCLC subjects. The complete response (CR) rate was 0/38 (0%), the confirmed partial response rate (PR) was 4/38 (10.5%), and the disease control rate (DCR) was 12/38 (31.6%).

36 patient samples were available for DLL3 expression analysis. Immunohistochemistry analysis showed that DLL3 expression was detected in 56% (18/32-4 samples were non-evaluable) of treated patient samples. Among DLL3+ patient samples, the overall response rate (ORR) was 22.2% (4/18), and DCR was 55.6% (10/18).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

SEQUENCES

| SEQ ID NO: | Sequence |
|---|---|
| 1 | SGVTCADGPCFNGGLCVGGADPDSAYICHCPPGFQGSNCEKRVDR |
| | CSLQPCRNGGLCLDLGHALRCRCRAGFAGPRCE |
| 2 | SGVTCADGPCFNGGLCVGGADPDSAYICHCPPGFQGSNCE |
| 3 | DLDDCAGRACANGGTCVEGGGAHRCSCALGFGGRDCR |
| 4 | RADPCAARPCAHGGRCYAHFSGLVCACAPGYMGARCE |
| 5 | GGGG |
| 6 | GGGGS |
| 7 | GGGGQ |
| 8 | SGGGGS |
| 9 | PGGGGS |
| 10 | PGGDGS |
| 11 | GGGGSGGGS |
| 12 | GGGGSGGGGS |
| 13 | GGGGSGGGGSGGGGS |
| 14 | QVQLQESGPGLVKPSETLSLTCTVSGGSISSYYWSWIRQPPGKCLE |
| | WIGYVYYSGTTNYNPSLKSRVTISVDTSKNQFSLKLSSVTAADTAV |
| | YYCASIAVTGFYFDYWGQGTLVTVSSGGGGSGGGGSGGGGSEIVL |
| | TQSPGTLSLSPGERVTLSCRASQRVNNNYLAWYQQRPGQAPRLLIY |
| | GASSRATGIPDRFSGSGSGTDFTLTISRLEPEDFAVYYCQQYDRSPL |
| | TFGCGTKLEIK |
| 15 | EVQLVESGGGLVQPGGSLKLSCAASGFTFNKYAMNWVRQAPGKG |
| | LEWVARIRSKYNNYATYYADSVKDRFTISRDDSKNTAYLQMNNL |
| | KTEDTAVYYCVRHGNFGNSYISYWAYWGQGTLVTVSSGGGGSGG |
| | GGSGGGGSQTVVTQEPSLTVSPGGTVTLTCGSSTGAVTSGNYPNW |
| | VQQKPGQAPRGLIGGTKFLAPGTPARFSGSLLGGKAALTLSGVQPE |
| | DEAEYYCVLWYSNRWVFGGGTKLTVL |
| 16 | QVQLQESGPGLVKPSETLSLTCTVSGGSISSYYWSWIRQPPGKCLE |
| | WIGYVYYSGTTNYNPSLKSRVTISVDTSKNQFSLKLSSVTAADTAV |
| | YYCASIAVTGFYFDYWGQGTLVTVSSGGGGSGGGGSGGGGSEIVL |
| | TQSPGTLSLSPGERVTLSCRASQRVNNNYLAWYQQRPGQAPRLLIY |
| | GASSRATGIPDRFSGSGSGTDFTLTISRLEPEDFAVYYCQQYDRSPL |
| | TFGCGTKLEIKSGGGGSEVQLVESGGGLVQPGGSLKLSCAASGFTF |
| | NKYAMNWVRQAPGKGLEWVARIRSKYNNYATYYADSVKDRFTIS |
| | RDDSKNTAYLQMNNLKTEDTAVYYCVRHGNFGNSYISYWAYWG |
| | QGTLVTVSSGGGGSGGGGSGGGGSQTVVTQEPSLTVSPGGTVTLT |
| | CGSSTGAVTSGNYPNWVQQKPGQAPRGLIGGTKFLAPGTPARFSG |
| | SLLGGKAALTLSGVQPEDEAEYYCVLWYSNRWVFGGGTKLTVLG |
| | GGGDKTHTCPPCPAPELLGGPSVFLFPPKPKDTLMISRTPEVTCVVV |
| | DVSHEDPEVKFNWYVDGVEVHNAKTKPCEEQYGSTYRCVSVLTV |
| | LHQDWLNGKEYKCKVSNKALPAPIEKTISKAKGQPREPQVYTLPPS |
| | REEMTKNQVSLTCLVKGFYPSDIAVEWESNGQPENNYKTTPPVLD |
| | SDGSFFLYSKLTVDKSRWQQGNVFSCSVMHEALHNHYTQKSLSLS |
| | PGGGGSGGGGSGGGGSGGGGSGGGGSGGGGSDKTHTCPPCPAPEL |
| | LGGPSVFLFPPKPKDTLMISRTPEVTCVVVDVSHEDPEVKFNWYVD |
| | GVEVHNAKTKPCEEQYGSTYRCVSVLTVLHQDWLNGKEYKCKVS |
| | NKALPAPIEKTISKAKGQPREPQVYTLPPSREEMTKNQVSLTCLVK |
| | GFYPSDIAVEWESNGQPENNYKTTPPVLDSDGSFFLYSKLTVDKSR |
| | WQQGNVFSCSVMHEALHNHYTQKSLSLSPGK |
| 17 | MDMRVPAQLLGLLLLWLRGARCQVQLVESGGGAVQPGRSLRLSC |
| | AASGFTFSNYGMHWVRQAPGKGLEWVAVISHHGSSKYYARSVKG |
| | RFTISRDNSKNTLYLEMNSLRAEDTAVYYCARDWWELVFDYWGQ |
| | GTLVTVSSASTKGPSVFPLAPSSKSTSGGTAALGCLVKDYFPEPVTV |
| | SWNSGALTSGVHTFPAVLQSSGLYSLKSVVTVPSSSLGTQTYICNV |
| | NHKPSNTKVDKKVEPKSCDKTHTCPPCPAPELLGGPSVFLFPPKPK |
| | DTLYITREPEVTCVVVDVSHEDPEVKFNWYVDGVEVHNAKTKPCE |
| | EQYGSTYRCVSVLTVLHQDWLNGKEYKCKVSNKALPAPIEKTISK |
| | AKGQPREPQVYTLPPSREEMTKNQVSLTCLVKGFYPSDIAVEWESN |
| | GQPENNYDTTPPVLDSDGSFFLYSDLTVDKSRWQQGNVFSCSVMH |
| | EALHNHYTQDSLSLSPGK |
| 18 | MDMRVPAQLLGLLLLWLRGARCDIVMTQTPLSLSVTPGQPASISCK |
| | SSQSLLHSDGKTFLYWYLQKPGQPPQLLIYEVSNRFSGVPDRFSGS |
| | GSGTDFTLKISRVEAEDVGVYYCLQGIHLPFTFGPGTKVEIKRTVAA |
| | PSVFIFPPSDEQLKSGTASVVCLLNNFYPREAKVQWKVDNALQSGN |
| | SQESVTEQDSKDSTYSLESTLTLSKADYEKHKVYACEVTHQGLSSP |
| | VTKSFNRGEC |
| 19 | MDMRVPAQLLGLLLLWLRGARCEVQLVESGGGLVQPGGSLKLSC |
| | AASGFTFNKYAINWVRQAPGKGLEWVARIRSKYNNYATYYADAV |
| | KDRFTISRDDSKNTVYLQMNNLKTEDTAVYYCARAENIGTSYISY |
| | WAYWGQGTLVTVSSASTKGPSVFPLAPSSKSTSGGTAALGCLVKD |
| | YFPEPVTVSWNSGALTSGVHTFPAVLQSSGLYSLESVVTVPSSSLGT |
| | QTYICNVNHKPSNTKVDKKVEPKSCDKTHTCPPCPAPELLGGPSVF |
| | LFPPKPKDTLYITREPEVTCVVVDVSHEDPEVKFNWYVDGVEVHN |
| | AKTKPCEEQYGSTYRCVSVLTVLHQDWLNGKEYKCKVSNKALPA |
| | PIEKTISKAKGQPREPQVYTLPPSRKEMTKNQVSLTCLVKGFYPSDI |
| | AVEWESNGQPENNYKTTPPVLKSDGSFFLYSKLTVDKSRWQQGNV |
| | FSCSVMHEALHNHYTQKSLSLSPGK |
| 20 | MAWALLLLTLLTQGTGSWAQTVVTQEPSLTVSPGGTVTITCGSSTG |
| | AVTSGNYPNWVQKKPGQAPRGLIGGTKFLAPGTPARFSGSLSGGK |
| | AALTLSGVQPEDEAEYYCVLWYSNRWVFGSGTKLTVLGQPKAAP |
| | SVTLFPPSSEELQANKATLVCLISDFYPGAVTVAWKADSSPVKAGV |
| | ETTTPSKQSNNKYAAKSYLSLTPEQWKSHRSYSCQVTHEGSTVEKT |
| | VAPTECS |
| 21 | MDMRVPAQLLGLLLLWLRGARCQVQLQESGPGLVKPSETLSLTCT |
| | VSGGSISSYYWSWIRQPPGKGLEWIGYVYYSGTTNYNPSLKSRVTI |
| | SVDTSKNQFSLKLSSVTAADTAVYYCASIAVTGFYFDYWGQGTLV |
| | TVSSASTKGPSVFPLAPSSKSTSGGTAALGCLVKDYFPEPVTVSWN |
| | SGALTSGVHTFPAVLQSSGLYSLKSVVTVPSSSLGTQTYICNVNHK |
| | PSNTKVDKKVEPKSCDKTHTCPPCPAPELLGGPSVFLFPPKPKDTLY |
| | ITREPEVTCVVVDVSHEDPEVKFNWYVDGVEVHNAKTKPCEEQYG |
| | STYRCVSVLTVLHQDWLNGKEYKCKVSNKALPAPIEKTISKAKGQ |
| | PREPQVYTLPPSREEMTKNQVSLTCLVKGFYPSDIAVEWESNGQPE |
| | NNYDTTPPVLDSDGSFFLYSDLTVDKSRWQQGNVFSCSVMHEALH |
| | NHYTQDSLSLSPGK |
| 22 | MDMRVPAQLLGLLLLWLRGARCEIVLTQSPGTLSLSPGERVTLSCR |
| | ASQRVNNNYLAWYQQRPGQAPRLLIYGASSRATGIPDRFSGSGSGT |
| | DFTLTISRLEPEDFAVYYCQQYDRSPLTFGGGTKLEIKRTVAAPSVF |
| | IFPPSDEQLKSGTASVVCLLNNFYPREAKVQWKVDNALQSGNSQE |
| | SVTEQDSKDSTYSLESTLTLSKADYEKHKVYACEVTHQGLSSPVTK |
| | SFNRGEC |
| 23 | MDMRVPAQLLGLLLLWLRGARCQVQLVESGGGAVQPGRSLRLSC |
| | AASGFTFSNYGMHWVRQAPGKGLEWVAVISHHGSSKYYARSVKG |
| | RFTISRDNSKNTLYLEMNSLRAEDTAVYYCARDWWELVFDYWGQ |
| | GTLVTVSSASTKGPSVFPLAPCSRSTSESTAALGCLVKDYFPEPVTV |
| | SWNSGALTSGVHTFPAVLQSSGLYSLKSVVTVPSSSLGTKTYTCNV |
| | DHKPSNTKVDKRVESKYGPPCPPCPAPEAAGGPSVFLFPPKPKDTL |
| | MISRTPEVTCVVVDVSQEDPEVQFNWYVDGVEVHNAKTKPREEQF |
| | NSTYRVVSVLTVLHQDWLNGKEYKCKVSNKGLPSSIEKTISKAKG |
| | QPREPQVYTLPPSQEEMTKNQVSLWCLVKGFYPSDIAVEWESNGQ |
| | PENNYKTTPPVLDSDGSFFLYSRLTVDKSRWQEGNVFSCSVMHEA |
| | LHNHYTQKSLSLSLGK |
| 24 | MDMRVPAQLLGLLLLWLRGARCEVQLVESGGGLVQPGGSLKLSC |
| | AASGFTFNKYAINWVRQAPGKGLEWVARIRSKYNNYATYYADAV |
| | KDRFTISRDDSKNTVYLQMNNLKTEDTAVYYCARAGNFGSSYISY |
| | WAYWGQGTLVTVSSASTKGPSVFPLAPSSKSTSGGTAALGCLVKD |
| | YFPEPVTVSWNSGALTSGVHTFPAVLQSSGLYSLESVVTVPSSSLGT |
| | QTYICNVNHKPSNTKVDKKVEPKSCDKTHTCPPCPAPELLGGPSVF |
| | LFPPKPKDTLYITREPEVTCVVVDVSHEDPEVKFNWYVDGVEVHN |
| | AKTKPCEEQYGSTYRCVSVLTVLHQDWLNGKEYKCKVSNKALPA |
| | PIEKTISKAKGQPREPQVYTLPPSRKEMTKNQVSLTCLVKGFYPSDI |
| | AVEWESNGQPENNYKTTPPVLKSDGSFFLYSKLTVDKSRWQQGNV |
| | FSCSVMHEALHNHYTQKSLSLSPGK |
| 25 | MDMRVPAQLLGLLLLWLRGARCEVQLVESGGGLVQPGGSLKLSC |
| | AASGFTFNKYAINWVRQAPGKGLEWVARIRSKYNNYATYYADAV |
| | KDRFTISRDDSKNTVYLQMNNLKTEDTAVYYCARAENIGTSYISY |
| | WAYWGQGTLVTVSSASTKGPSVFPLAPCSRSTSESTAALGCLVKD |
| | YFPEPVTVSWNSGALTSGVHTFPAVLQSSGLYSLESVVTVPSSSLGT |
| | KTYTCNVDHKPSNTKVDKRVESKYGPPCPPCPAPEAAGGPSVFLFP |
| | PKPKDTLMISRTPEVTCVVVDVSQEDPEVQFNWYVDGVEVHNAKT |
| | KPREEQFNSTYRVVSVLTVLHQDWLNGKEYKCKVSNKGLPSSIEK |
| | TISKAKGQPREPQVYTLPPSQEEMTKNQVSLSCAVKGFYPSDIAVE |
| | WESNGQPENNYKTTPPVLDSDGSFFLVSRLTVDKSRWQEGNVFSC |
| | SVMHEALHNHYTQKSLSLSLGK |
| 26 | EVQLVESGGGLVQPGGSLKLSCAASGFTFNKYAINWVRQAPGKGL |
| | EWVARIRSKYNNYATYYADQVKDRFTISRDDSKNTAYLQMNNLK |
| | TEDTAVYYCVRHANFGNSYISYWAYWGQGTLVTVSSGGGGSGGG |
| | GSGGGGSQTVVTQEPSLTVSPGGTVTLTCASSTGAVTSGNYPNWV |
| | QQKPGQAPRGLIGGTKFLVPGTPARFSGSLLGGKAALTLSGVQPED |
| | EAEYYCTLWYSNRWVFGGGTKLTVLGGGGSGGGSEVQLVESGGG |
| | LVQPGNSLRLSCAASGFTFSKFGMSWVRQAPGKGLEWVSSISGSG |
| | RDTLYADSVKGRFTISRDNAKTTLYLQMNSLRPEDTAVYYCTIGGS |
| | LSVSSQGTLVTVSSGGGGSGGGSEVQLVESGGGLVQPGGSLTLSCA |
| | ASSSSVSLLSLAWYRQAPGKKRELVAGISDDGSIVYMDSVKGRFTI |
| | SRDNAKNSVYLQMNSLRAEDTAVYYCYAYSWITRSPYWGQGTLV |
| | TVSSHHHHHH |
| 27 | EVQLVESGGGLVQPGGSLTLSCAASSSSVSLLSLAWYRQAPGKKR |
| | ELVAGISDDGSIVYMDSVKGRFTISRDNAKNSVYLQMNSLRAEDT |
| | AVYYCYAYSWITRSPYWGQGTLVTVSSGGGGSGGGSEVQLVESG |
| | GGLVQPGNSLRLSCAASGFTFSKFGMSWVRQAPGKGLEWVSSISG |
| | SGRDTLYADSVKGRFTISRDNAKTTLYLQMNSLRPEDTAVYYCTIG |
| | GSLSVSSQGTLVTVSSGGGGSGGGSEVQLVESGGGLVQPGGSLKLS |
| | CAASGFTFNKYAINWVRQAPGKGLEWVARIRSKYNNYATYYADQ |
| | VKDRFTISRDDSKNTAYLQMNNLKTEDTAVYYCVRHANFGNSYIS |
| | YWAYWGQGTLVTVSSGGGGSGGGGSGGGGSQTVVTQEPSLTVSP |
| | GGTVTLTCASSTGAVTSGNYPNWVQQKPGQAPRGLIGGTKFLVPG |
| | TPARFSGSLLGGKAALTLSGVQPEDEAEYYCTLWYSNRWVFGGGT |
| | KLTVLHHHHHH |
| 28 | FPVHPDGASALPAAPPGLRPGDPQRYL |

1. A method of treating small cell lung cancer (SCLC) in a subject, which method comprises administering to the subject a bispecific antigen-binding molecule comprising at least a first binding domain that binds to human delta-like ligand 3 (DLL3), wherein at least 25% of the SCLC cells express DLL3.

2. A The method of claim 1, wherein at least 50% or at least 75% of the SCLC cells express DLL3.

3. (canceled)

4. The method of claim 1, wherein DLL3 expression is determined using an immunohistochemical (IHC) assay.

5. The method of claim 4, wherein DLL3 expression is determined using Formalin Fixed Paraffin Embedded (FFPE) tissue specimens from the subject.

6. The method of claim 1, wherein the SCLC has progressed or recurred in the subject following a platinum-based treatment.

7. The method of claim 1, wherein the SCLC has progressed or recurred in the subject following a platinum-based treatment in combination with etoposide, and optionally a PD-L1 inhibitor.

8. (canceled)

9. The method of claim 1, which further comprises administering to the subject a PD-L1 inhibitor and optionally a chemotherapeutic agent.

10. The method of claim 1, wherein the bispecific antigen-binding molecule comprises a second binding domain that binds to human CD3.

11. (canceled)

12. The method of claim 10, wherein the bispecific antigen-binding molecule comprises an antibody, a single chain variable fragment (scFv), tandem single-chain variable fragments (scFv)₂, a bispecific T cell engager (BiTE®) molecule, or a heteromultimer.

13. The method of claim 12, wherein the bispecific antigen-binding molecule comprises the amino acid sequences of SEQ ID NO: 14 and SEQ ID NO: 15.

14. The method of claim 13, wherein the bispecific antigen-binding molecule is tarlatamab.

15. The method of claim 12, wherein the bispecific antigen-binding molecule comprises a first heterodimer that binds to human DLL3 and a second heterodimer that binds to human CD3, wherein
(a) the first heterodimer comprises a heavy chain (HC) comprising the amino acid sequence of SEQ ID NO: 17 and a light chain (LC) comprising the amino acid sequence of SEQ ID NO: 18; and
(b) the second heterodimer comprises a heavy chain comprising the amino acid sequence of SEQ ID NO: 19 and a light chain comprising the amino acid sequence of SEQ ID NO: 20.

16.-18. (canceled)

19. A method of treating SCLC in a subject, which method comprises administering to the subject a bispecific antigen-binding molecule comprising at least a first binding domain that binds to human delta-like ligand 3 (DLL3), wherein the SCLC has a DLL3 expression level in which at least 25% of the SCLC cells express DLL3 at an intensity equal to or greater than 2+ as determined by an IHC assay.

20. The method of claim 19, wherein the SCLC has a DLL3 expression level in which at least 50% or at least 75% of the SCLC cells express DLL3 at an intensity equal to or greater than 2+ as determined by an IHC assay.

21. The method of claim 19, wherein at least 25% of the SCLC cells express DLL3 at an intensity of 2+ or 3+ as determined by the IHC assay.

22. (canceled)

23. The method of claim 19, wherein the SCLC has progressed or recurred in the subject following a platinum-based treatment.

24. The method of claim 19, wherein the SCLC has progressed or recurred in the subject following a platinum-based treatment in combination with etoposide, and optionally a PD-L1 inhibitor.

25. (canceled)

26. The method of claim 19, which further comprises administering to the subject a PD-L1 inhibitor and optionally a chemotherapeutic agent.

27. The method of claim 19, wherein the bispecific antigen-binding molecule comprises a second binding domain that binds to human CD3.

28. (canceled)

29. The method of claim 27, wherein the bispecific antigen-binding molecule comprises an antibody, a single chain variable fragment (scFv), tandem single-chain variable fragments (scFv)₂, a bispecific T cell engager (BiTE®) molecule, or a heteromultimer.

30. The method of claim 29, wherein the bispecific antigen-binding molecule comprises SEQ ID NO: 14 and SEQ ID NO: 15.

31. The method of claim 30, wherein the bispecific antigen-binding molecule is tarlatamab.

32. The method of claim 29, wherein the bispecific antigen-binding molecule comprises a first heterodimer that binds to human DLL3 and a second heterodimer that binds to human CD3, wherein
(a) the first heterodimer comprises a heavy chain (HC) comprising the amino acid sequence of SEQ ID NO: 17 and a light chain (LC) comprising the amino acid sequence of SEQ ID NO: 18; and
(b) the second heterodimer comprises a heavy chain comprising the amino acid sequence of SEQ ID NO: 19 and a light chain comprising the amino acid sequence of SEQ ID NO: 20.

33.-36. (canceled)

37. The method of claim 1, wherein the bispecific antigen-binding molecule is tarlatamab, and wherein tarlatamab is administered at a dose of from 10 mg to 100 mg once every two weeks.

38. The method of claim 37, wherein tarlatamab is administered at a dose of 10 mg once every two weeks or 100 mg once every two weeks.

39. (canceled)

40. The method of claim 37, which comprises administering tarlatamab at a dose of 10 mg to 100 mg once a week on weeks 1, 2, and 3 prior to administering tarlatamab once every two weeks.

41. The method of claim 1, wherein the bispecific antigen-binding molecule is tarlatamab, and wherein tarlatamab is administered at a dose of from 10 mg to 100 mg twice every three weeks.

42. The method of claim 41, wherein tarlatamab is administered at a dose of 10 mg, 30 mg, or 100 mg twice every three weeks.

43. The method of claim 41, wherein tarlatamab is administered on day 1 and day 8 of a 21-day cycle.

44. The method of claim 1, wherein the bispecific antigen-binding molecule is tarlatamab, and wherein tarlatamab is administered at a dose of from 20 mg to 200 mg once every three weeks.

45. The method of claim 44, wherein tarlatamab is administered at a dose of from 20 mg to 100 mg once every three weeks or from 100 mg to 200 mg once every three weeks.

46. (canceled)

47. The method of claim 44, wherein tarlatamab is administered at a dose of 20 mg, 60 mg, 100 mg, or 200 mg.

48. The method of claim 44, wherein tarlatamab is administered on day one of a 21-day cycle.

49. The method of claim 44, which comprises administering tarlatamab at a dose of 10 mg to 100 mg once a week on weeks 1 and 2 prior to administering tarlatamab once every three weeks.

50. (canceled)

51. The method of claim 1, wherein the subject is a human.

52. The method of claim 1, wherein the subject had at least one prior treatment of the cancer and relapsed.

53. (canceled)

54. A method of treating neuroendocrine prostate cancer (NEPC) in a subject, which method comprises administering to the subject a bispecific antigen-binding molecule comprises a first binding domain that binds to human delta-like ligand 3 (DLL3) and a second binding domain that binds to human CD3, wherein the NEPC cells express DLL3 as determined by an immunohistochemical (IHC) assay.

55. The method of claim 54, wherein the IHC assay is a Ventana SP347 IHC assay.

56. The method of claim 54, wherein the bispecific antigen-binding molecule is a protein that comprises the amino acid sequences of SEQ ID NO: 14 and SEQ ID NO: 15.
